SEQUENCE LISTING (246 sequences)

SEQ ID NO: 1 - DNA, 38444 bp, Homo sapiens
  source 1..38444: mol_type = other DNA
agtcagccctgctgccagccagtgccgggtgctggggactcagggaggcccgccgggaccactgcgggacagtgagccgagcagaagctggaacgcaggagaggaaggagagggggcggtcagggctctcaggagccgggtcctgggcaaggcgcagccgttttcaaattttcaggaaagcggtcggctcacactcgagcagtaaaaagatgcctctggggaggaggcccgtgcagctctccgggcaatggtggtggctcggcctagagaggcggtagtggaacgcagaccctggtgggggaatgacatcaagggaggagacgggcgggaccccagatttctgcctgtgggcgatggaagtgaggttcactggccagcggagccggacacagaacgcgcaaaacgccgtgtaggcctggaggagccgaagagcaggcggaccccctccgcgggggaacagtttccgccgggagcacaaagcaacggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtgagtcgcagtgaccctacaagtggttcttttacccgagcggctcgtaggcgcgttgcggtttttcgaaactacagctcccggcaggccccaagccgccctcggggccgcgggtcggcggattggccgcgctgcattttgggacctgtagtttcctgcgctcgtggcgctggcgccgcggcgttggctgagcccttgaccggggctggagggaagggccgacattcagtgtgtccgcgtctgttctgttagtcccagttcccgggcgggattgaggcttagagaagttgagtgatttgctgagggctgcacgggttggcatcccggcatgctctttcgctactttggctgcatctggttgcccacccgggcggatggggaatggactccagccagccaggagggcagagggctggagaggcagggccggaggttcagaccctccgctctgacgttgcgcctggtgaggccgggaggggtgccgcttgcctcttcagccctcacgctcttgtggaagtcgcggaattactgcaggcggaacttgcagcactgtgggcgtcttttccagagaaggacggagttgtggggcgggaggataaggcaaggcccagccacttcgcatcttcgccccgccagctcctcgagatgggatataccagggttgctctccaaccctctccgcaggagggactgatggaaacgcctgggaaagtagcccggtacccacaaaggctgtctacaaacagagtcttactgtctttcccaggtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcaggtagagcacaggccccgaggaaaggactgcgggtgggtggagcttcagccaggacggggtgtgcttccctctcccggcccattccagccaggcccctccgggccagaggcagcgtctgtcataaaaagggctggtgttccaggtggggtcagagagaggattgacaagtaaaaacgatcgtcctttgaagggggccggcccctccacacctgtgggtatttctcatcaggcgggacgagagactgagaaaatgaataagacacagagacaaagtatagagagaaaagtgggcccaggggaccggcgctcagcatacagaggacctgcaccggcaccagtctctgagtttcctcagtattcattaattactattttcactatctcagcaagaggaatgcggcaggacagcaaggtgatagtggggagaaggtcagcaagaaaacgtgagcaaaggaatctgggtcacaaataagttcaagggaaggtactatgcctggatgtgcacgtaggctagttttatgcttttctccacccaaacatctcggtggagtaaagagtaacagagcagcattgctgccaatatgtctcgcctcctgccacagggcggcttttctcctatctcagaattgaacaaatgtacaatcgggttttataccgaaacattcagttcccaggggcaggcaggagacagtggccttcctctatctcgactgcaagaggctttcctcttttactaatcctcagcacagacccttcacgggtgttgggctgggggactgtcaggtctttcccatcccacgaggccatatttcagactatcacatggagagaaaccttgggcaatacccggctttccagggcagaggtccctgcggctttccgcagtgcatcgtgcccctggtttatcgagactggagaatggcgatgacttttaccaagcatactgcctgtaaacatattgttaacaaggcacgttctgcacagctctagatcccttaaaccttgattccatacaacacatgtttctgtgagctcaaggctggggcaaagttacagattaacagcatcttagggcaaagcaattgttcagggtacaggtcaaaatggagtgtgttatgtcttccctttctacatagacacagtaacagtctgatctctcttttccctacagtccttgagggtgacagacttaggagtgccttgggggcctctctgaggagcagctgatattcacgggtcaggaggaagcatttccattagaggggcagccggtggccagcctcacttggaaggtctttgaacctcgggggtgcagggaggtggcagtggtgcaggttgccttctcctgggttccttgaggtgccctcttgtacccggctcacacccttcccctccccgagtttcctgctcaggttcccgtctgagagcttgtatgtaggacgtcagataggacagcataaatgtttggatccagaaacgcagaacagtttcctattttgagacttgacacctaattagtcatcttactatttaagctgaaaaatagtgtcgtgttttgggtaacgttctgcaaatcgtttgctaatggcggctgagttgcttcacgccctttagggcaagagtgggacttgcctgtggacttctccgcggtcccacagggctctcgccacctggcagtggcctctgcatctgcaaagagctgcccgctggctgccgaagcttgtctcagggcagcttgtgtggcctcgcctcttcctggcttccccgtaacccttgctccgaactccgttcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccataggtgaccctagttcccaggcctctcctggcctcctgtggggatggttggcaagggatggcgctgagggtggggtgggcccatggggactcctgccgtctctcaagcagaactcaaggagaattttttagctgctgtataatttctcgccatcgtgggtgtaaacctagggttgggcttttttgctgaattagggcacggcagatgcccacttcacccatttttgataaaccagtatctggggtgtcagattcttggctgtctgcagggccgagttagccgaatgccacctgcctttgatacgtgagaacgttgtctgagaaccgtgacttctgtgcttgcttgtgtctggtcagcttgctacacggacatcccaaagattatttacgcctccaggacccactcgcaactcacacaggtcatcaacgagcttcggaacacctcctaccggtgggtcagacgagtttacacctgtctcggggtcctcaagagaaccagcttggcatggtgctgagtccacagccccatgctgtgctgtggtggagggtggtggtctttctagacgctcccccgaagtgtgcagagcgctggtgcccaggggtggggtgcggcctgggctgcctccaatgcccattacttgtgaggaagcagctttgcatctgtgtgctgaccttgggcgggcgtcctgagctcctcgcaggtgctgttgtagcagctgtgcagtaggtcagggctggcccccagtgcagctttgcacatgaagtaggaggaggccctgctgcttgtcagagcccagcagagtcttggtgttctgtcgggttcctgtggccggaccagtggcagggtgctgtggaagctgtcgaatctcctccctctgtccagtacccccgctcgtcttctagctccctcctacgcccgggccacgtttcagttatgctcacttcctctgaccgccgaggctcctgcgtgtctccatacagctcacgctgcagggccacgctgtgggtgttggagacagctcctcctcgacccacggtgctctctcccaccaggcctaaggtgtgtgtgctgggctcccgggagcagctgtgcatccatcctgaggtgaagaaacaagagagtaaccatctacaggtaggctcctgggctcccgctccggctcagtgtccgacaggcgagtgctgctgggtgtccagagccccaggctgcgctcccgctgggctagggtttgaagttcactgggggactgcaggggaggacctggtgggggtggggactggcttcggtcctttcttggccgtgcttcagctgcgcactctgcccttcctcccacagatccacttgtgccgtaagaaggtggcaagtcgctcctgtcatttctacaacaacgtagaaggtacaagcagctgggtgggaccagggtcgggttggagtgtgtgcagcctctcagggtggagctcagtggtgtcacagcctggttgtgcttgcccggtggggcggccagtgcggccatgtacctgggccctgtcttctgactcggggccacccatgttagacttctgtgtggaagagctcacacagtggtctgagacagccagccggcaagactgcctctggctggtgcctggggccttggattttgggaaggctccctccatttcctgatgagagggtctccctgcacctaacctgctggtgcaaacagtaggggttttgctgaacaccggctttctcttcggggactttgttgcttgcccagcagcaggtgctccagtgaccggccctcataccatcttgggagggtgtcctggaagccgtgtctggcctcccgcgaccctgccccgtgtgtctttttcctgtgctgaccttgctgcggaaaattatggccctgagtgtgactccaggctgagtcctgtgggtccaacacgggatgccttggggcctcttctggagacgggatgtgagtgacaggagccggccggggcagcttgccctgtgactgcacgtggccacagcctgtgagggccgggggtgcttctccacccacgtggctgcccctcgggtatgtcaagggcttctggggctcatcacggggtcctagagacagtggcagggtgcacccccgttggctgcccttacagtttctgtgacctgagggtggcatctgtgcagtcggcgcggtctgtgcttctgtgggatcagggttccctctgtttcctgcctcagttggggctcaagcctcaggtgaggtggccccggagcactcagaaggcatcggcggtcctgtgggctgctttctgcactcacgtttgctgagtgctcagtgtgccaggactgaggaccctgaagctgctcttgtatttagggcggcgctcccctggcagagactgagccaggtggtcccgcatgacccactaccaggcgtttctgggccctggcccttggagggacagggtgggcggaacatgggcctgcagggaggctcccgcttactggaggcatgtgctgtgttgctggagacatcctctgtgttgcttcttgttcgctgtggtttttggtctggtggcaccaaggaccctcagtcatcttgatgtgtggttgtccaggcctttttgttggtcctaagaaggggctctgcctttgtgcccccaggttccctgacaggagctgccggctcgtcccggtgatgcctgcaggacgtgactctgggacggggggttgggcagatgtgctgatggaaattctcaagcaggcgtcatttccgaggtcctcacctggatttccaggacaggagtgcctgctgggtgtccccagtcccatgcagcgggggtccttgggatagcatggaacgctgagcatgggcctggccggccgtggtcctggacaagggcagtgccccggtggctgctgggcctgggacctggtggggacgctgggcctggtacctggtggggatgctgggcctgggacctggtggggaggcctctgactgcctcctggtgctgcttccgtctgtgttaggcctctgggtattggggcccccatctgtctcctcctccaggcctgtggactcagaccaggaagacacaggccagcccctgcctgtcccccttggcttgggctctcactgcccgacctggcgggaggttgcctagccgtgaaccttcgcaccctgtctgccaccggacaggctgtgagggggtgtctgcagcacctgcaccggcctgagcatcttcagagtgggctgcagctcctggaggggtctgagaggaagggaggcaggtattttgggcgaatgaggagacagctggagagctggcacccttcctggcctgcgtcctgtgaggactctggttggggacagcaagcttggggtcagcctggggcagagcctctgggacggccccgcccctcgtgccccttcccctcgcagctcctgtcctcgccccgccctcagctctccgccaggcaaggtttggcaagtgccgctgtgcggcagtgcctgctgattggctggtctgttgctatggtgctgcccaggggtgtgcttttcctcccctgccttccctgctatccctgggagtatctggggttgggtcatcgctggtgtgtgtgagtgtgtgtgtgtgtgtatgtgcacgtgtgcatatgtgtgcgcttctggcctctgcagctgagtcctggccctcggggggcctggcacctcctggggacaggcacaaagcagccatgatggagtcgggagctgggggaggccccattgccccacgtggctgccctgtgactctggggtgcttgttagaagaggtatctggttctgtctgtgtttaagcaactccctaaggaattcttgtggttccagtttggggggcctgtactgtagaggcaagggaggggcaggacatcccccagactctgacttctgaagccttttctgcccggggcctctccgccagtacaggcagtgtcctttgccagggctgccatgctgcagaggggagtgggccactgtttagcccaggaaaacctggctctcccttagctggaagttctgggcctgttgtggttggcagggaagctgagtgacggtgctaatcacaggggcacctgcaggggtttgtgggagatgcctctgtgggttggggcgataggctgaggggctgttcttccctgccctgaggagggctgagtgtagccgccactcctgtcctgtcttgggctgtctcggagaggatgcgtagaaccctcgggatcctgctggcctccgtctggtccaccctgaacctcaggccttctgggggcagaggaggattccctcaggatcactcgggtgggggcctctcttgggcacctgagaccctcagtgggtgctttgtggcgcgttcacggttggtgggggacgcccagccctgcccgccgtgtaggagccgttctgtcctgggcatccccctgtggtctgggacttagtggaccctgagggtgtgtgtttacccctgcctcacacctgcagaaaaaagcctggagcaggagctggccagccccatcctggacattgaggacttggtcaagagcggaagcaagcacaggtgagacccctcagtgaggccacgaccactgtccttccatggcccagctctcctgtgacctgtggaggcccggatatatttcttcacttttctttgttcctttttaaattatgaaactaaccaccattcagtacgaaaaagtttaagcagctctgaggaagatagagtaaaaaattgtctccctcttccctggccctcagccatccccggtggccaccgtggagtgtggacggagccctgcaggcctgtgtctgtgcggaagcacgcgcagttttgtctgcacagactgtcctgcagttggctgttttcactcagcgttgtgggtatagcttcccatgctggtgctggcagctcggccttgttcttttgaggacagcagatgtctcctatgtctacctcttacagcttcagagattcaagttataataaagctcttcttatattgagggggaaacctccctcccccttttttttgaaacagggtctcgctctgctacccaggctgcagtgcagtgtcacagtcttggctcactgcagcctcagcctcccaggctcaagcgattttcccacctcagcctcccaagtagccgggactgcaggcacacaccaccatgcctggttaatttttgtattttttgtacagacagggtctcactctgttgctcaggccagtctcctgagctcgagagttccacctgccttggcctcccaaagtgctgggattacaggcgtgagaccccatgcctggccagctctttttttttttttttttttttttgagacggagtctcgctctgtcgcccaggctggagtgcagtggtgcgatctcggctcactgcaagctccgcctcccgagttcacgccattctcctgcctcagcctcccgagtagctgggactacaggtgcccgccaccacgtctggctaattttctgtatttttagtagagacggggtttcaccgtgttagccaggatggtctcgatcttctgaccttgtgatccgcccacctcggcctcccaaagtgctgggattacaggagtgagccaccgcgcccggcccagctctgctttttcttagtggttctgcgttgtgtttgtttctatccaggaatagggttggttttacttttccatcgagtttttaaagagacgacgatttacatggtcggaaactcacgaggactccccatcccttggtcggaaactcacatggactccccatcccttggtcagaaactcacgtggactcccatccatcccaggcagcagcttcccacctgggccctacgtgcaggatgagggctccttccgggtcagaagacatggcggcctcggggcaccgtcccctgcatggggtgctcacaggatcttctcctctctccttcccagggtgtgcccttactacctgtcccggaacctgaagcagcaagccgacatcatattcatgccgtacaattacttgttggatgccaaggtgggggctcagtcctgtagctgacgactcctgatgtccaggggtgtccctgggcttgggaacagctgtccgagcctttgctgcttcagggccttagatcagcaggcctgggtgggaggactcacctctgtcactgggcaggggctcaacctggccagacacacttgtgagcagccccaggccacaggtcagttttctgagcagtctgggagcgggcaggctggtgggagtgaggagagacctccaggctgtggtccataggccagtgcccgctcttgatcctgacagctcaggttctctccttcacgtcaggccatgggaggcaccgagaacacaggaagcccactgactcccctcttcccagcgcgtgcccggccccacactcactccccctcccagcatgtgcccggcttcacactcactcccctcttcccagtgcatgcccggccccacactcactccccccacagcatgtgcccggcctgacactcactcccctcctcccagtgtgtgcccagccccactcccttccgccccgtgtgcccagccccacgctcactccccccgccagcatgtgcccggccccacactcaactcccctcctcccagtgtgtgcccggccctgctgccctcctccccatgtgccctgcttttgtgccccacactttttacttagtgcaggtgggatcacacgccacgggtcaatggtttgtgtgttcacgtgacgatggcgtggtgacgtttccagatcccgtcgttggttcgctcattctcggggtgtatatttattgagagctcatcatgctgggtgctattccaggcatagcaagactggcttcactcacatggagctttgattctagtggtggggacaggtggacagcaaaagagtaagcacgtgagctgatgatactgaagggaaatagagcagagggaggaggcggagaccgagccaagcgggcccaagtgcgatgtcggcgggaggtggggaatgctggtgggtctgaggggagcctcagcaggtgcagcagagcaagggaagaggtgagtgggggcggctggggggccgactcctgggaagctgtagcagaaccccacagagagctggtgaggtttgccgtggttgtgggtgactcggtgctttgagccctggctgcccctgggaaccatctggagagcttctaacccaaccaggcccctccctgggacagttatatcacagctggtaagccgagtctaacactttcacggaaacgcagaagatctaaaacagcaagatgaccgtgaagaagaacagagctggaggactcacctcgctggtttcaagactcctctaaagctgcaggagtggaggtggagatggcccagctcaggcacaggcctgcaggccatggagaaggcagcaagctcaagctgacccacacgcatgtggtcattgttttttttttcagttggaatctcactctgtcacccaggttggagtgcagtggcaccatctcggctcactgcagcccccgcccctaggttctagcgattctcccacatcagcctcccgagtagctgggattacaggcgtgcgccaccatgcctggcccttggtgattgttttttgacaaacatgccaatttaattgagagaggaaatgaaggttgatttctggttttctgaaaaaatggtgctaagaacagctggatatctgttcggaaaacagtgaatcttaactcttgttttaccctgtataaacctaaatgtaaaagctaaactaaaagttatagaaaggaacatgggggaggtctttgcaactttggggtaggcagagatttcttagtatggatacacaaggcactagccatgaagaaaaacattaaaatttagacttcaccaaaatttaaagcttcaactctgtggaagagttgagaaaatgaaaaagcagttaaagaaagggagaaaatacttctttcaaaggacttaaaaaattttttcagccctcctctgatttgaaaggacctttgaccagagtatgtaaaattctcccataactaagcaaacaacccacttaaccactgggaagggatctggacagacgtttcaccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcaccaaggtggatggaatgaccagttgagcacatggaaagtcgcccagcatctccagtcataggagaaggcagattaaagccacggggagccgacactgtggtcccactggcatggctgaaattcagaagccctgagtgtggcatgaggatgtggaacagctggatctcatccatcgctgtgaagttgtgtagccactccacaaacgtgtggcaaacagccgagccgggagaagggaagacgtgttcaaagattcatatgtggccaggctcagtggctcacgcctgtaatcccagaactttaggggccaaggctgggggatcgcttaagcccaggagtttgagaccagcctaggcaacatagggagaccccatctcaaaaaaaaaaaaaaagaaaaaagaaaagacttcagtgtgcaggtttaccagagttttgtttgcagttgccaaaactgggaagcagcccgcgtgagcccatccacaggtgaatggacagaccgtggtacccgaacactaacagcagccacgggcgtggactgtggtcacacagcagcagggagccgatgagtctcggacatgctaacccagagaggcccattgaggaggacctactgttttttgtgtttttgttttttgttttgaaatggagtctcgctctgtggtgcaggctggagtgcagtggtgtggtcttggctcactgcagcttccgcctcttgggttcaaacagttctcctgcctcagccttccgagtagctgggactacaggcacccgccaccacacccggctaatttttgtattttcagtagagacggcagttcgccatgttggccaggctggtcccaaactcctgaccttgtcatccactcactttggcctcccaaagtgctgaggttgcaggcatgaaccaccgcacccggctggacctactgttttattccatttatgtgacactctattaatagaaaaggcaggggtggggctggtggttatatggtgcacataactgccagaactcagtacacttaaaatgaacatcttaatgtgtgaaatttttttttttgagacggggtcttgctctgtcacccaggctagagtgcagtggtgcgatctccactcactgcaagctctgcctcctgggttcacgccattctcctgcctcagcctcccgagtagctgggactacaggcgcccgccaccacgcctggctaattttttttttttttttgtatttttagtagagacggggtttcacagtgttcgccaggctggtctcgatctcctgacctcgtgatccgcctgcctcggcctccgaaagtgctgggcttgcaggcgtgagccaccatgcccggccaatgtgtgaaaatttaaaagtaccaaagctggaccccaccccagattgctcccatgacactctgtgggtgggacctgggagttgggttttgttttgttttgttttgtttttgagatgaagtctcactctgtcgcctaggctggagtgcagtgacacaatctcggctcacattaacctctgcctcccagatgaaagcgattctcctgcctcagccttctgagtagctgggattacaggcacacaccaccaccccctgctaatttttgtatttttagtagagacggggttttaccatgttggccaggctggtcttgaactcctgacctcgtgatccgcccgcctcggcctcccaaagtgctgggattacaggcgtgagccaccgcgcctggctgggagttgggtttgtaaatctccctgagtggggctggggcagggaactgctgggtctgggtcttcctggctcctctggtctgtggcttcctgactgcggtggccgggggctcccagggcatcgtggccgtctgtcttgctgagcgtggcacgtgcctttccatgctgtggaggagcgtctcccggtatggcgaactgctggttagggtggggcggtgttgccaggtcatccaggtctggcctctgctctcgacatcgccggcgctgttgctcatctgcgcttgtgatgttcgatgcctgctgcacatgtcttggcttccctctttcccggcctctgtgagctccagcgctgcgtcccttctcttcctcctgtagagccgcagagcacacaacattgacctgaaggggacagtcgtgatctttgacgaagctcacaacgtggtgagtctccgctggcctcctaaacacctcctattgcttctggcctttttgtcaagagccacgcaaacctttctggaggggctctggccaaactcctgaagccctaggtgcccaggactggggactgagcacaccaggagcttctgccaccccctcccgccctgatccgatgcctctgctggggctggagactggccagctgggccagggacctgcccgtcaggcgcagggcccccacaggccgctcaccagaccctttccctccagccagctcggggtcagcctgggccagggctgtctcctctgccctcggcagcagcaggcttgtggtcttgcctgcagtgtctctgcccttccggccacatggcttgagactgaggcaggagaatcgcttgaaccttggaggcagaggctgcagtgagccaggatcacaccactgcattccagcctgggtgacaaagcgggattctgtgtcaaaaaaaaaaatgttgactgggcgcgctagctcatgcctataatcccagcactttgggaggctgaggtgggcggatcacgaggtcaagagatcaagaccatcctggccaacatagtgaaacaccgtctctactaaaaatacaaaaaaattagctgggcgtggtggcgtgtgcctatagtcccagctactcaggaggctgaggcaggagaatcactcgaacccaggaggtagaggttgcaatgagccaagatcacaccactgtactccagcctggtgacagagcaagactccgtctcaaaaaaaataaaatcaaaaagaataattggcaattccagtgaaataattgtttgtttgtttgttgagacagggtctccttctgtcgtccaggctggagttcagtggtatgatcttggcccactgcaacctccacctcctgggctcaagccatcctcccacctcagcctcccgagtagccgggactacaggtgcacaccaccacgcccggctaatttttgtattttttgtagaggcggggtttcccagcgttgcccaggctggtcttgaacccctgagctcaagtgatctgcccaccttggcctcccaaagtgctgggattacaggtgtgagccaccgcgcccggcctgaaacaatcgtttctaaatattggtgtgggccacacagtcatgtttggacctacttgtggccttttacagaccccaggccaaggctttgggaacttggctgtcagcctcctgtgccttctgcacccccaccccatttctgctttctggaacccccgatcctgtcctgttctgtggtgattcgggtgtgcttgggctctaggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccaggtgcgttcatagccagactgcttggtcctgaggcctgcgctgctgcagggtgagccccacccggagttcagcacggactcccccagcccaggtgcgttcatagccaggctgcttggtcctgaggcccgtgctactgcagtgggcagcctgccctgtggctgtgtgtggtcggcctgggcaccatctattcaggctggcactgcagggcatccgcttctctcagaggcttcttgggtgtgaattcttcagggtcctgtagcctgtggaagggctggtattgttcagtagttctggtattttccaaagacctatgtcttctcccagccagtatcaacttggcctctactgtgtaaaactggaaaactctactttgtgaagctgagttgggagcatcgcttgaggccaggagtttgagaccagcctgggcaacatggcggaacctcgcccctgccaaaaaattagccaggtgtggtggtgtgctcctgtggtccaagcttttccggaggccgaagtgggaggcgtgcttgagcctgggaggcagagcttccggtgccccagatgactccactgcactccagcctgggcggcagagtgaggccatctcaaaaaaaaaaaaaaggaaaactaaatatattcactgtaagggcattttgcatctttaaatgacccacaaatctggcatgcatcagctgctctgcctgtaggttccttcccagtgtttgtccagaggtgtatttccacacagcgctagtcacggcatacgtggaaaacgtggaaacccttcatggatgttgtcagttggtctatattttctttcttttttttttttttgagatggagtttcacttttgttgcccaggctggagtgcaatggcgcgatcttggctcactgcaacctccgcctcctgggttcaagcaattctcctgcctcagcctcccaagtagctgggatcacaggcgtgcaccaccacgcccagctaattttgtatttttagtagagatggtttctccgtgttggccaggctggtctcgaactcctgacctcacgtgatccacccgcttcggcctcccaaagtgctgggattacaggcgtgagccgccacgcccggcctttgtccatattttctacatggcttctgtaaacagctgactaggagtctgtgtgaatatcttcataggttctgctgtgacactacttgctcgtgagcatctccaggtgtaaacagcatcagcttcccccattttcctttaaaatcgcacatgtggacggacaccacggggaccctggaccctggggagccccgtcctcacccttctcaccaggatggctgcttggtagagagtgagtttgcaaagttggcatttgtttagtacagaagttatcaggtgttctggctttagaatccctttatatatatatatatatacatatatttaagtgacagggtctcactctgttgcccaggctggaatgtggtggtacaatcaaagttccctgtagcctcggcctcctgggctcatgggatcttcccgtctcagcgtcttaaagcgccgggaccacaggtgtgcaccactgccaccggctctcaagattgccacgcagggagttgcagtgggggaaggggttcctgggactttgaacgctccacctccctcctctccacagtcccccaaccccacctctctaacggggtggacggccgcctctttccatccttcgcttggcgcagggtggggagagtgacaggtctccttccctcatctcggcagctgccatttcatcgcttacataacgtgggagaaacatccacccacccccaggcctgtgtgaacatcaccacggggccttctccactcttcagttttgttagttacttgatgtgcagggctttttgttgtaactagtgggggacgtgtggtggggtgggcttctgccatctcattcaggaccagaacttcagttttcatccctatctgttcccccacccctttggagatggggtctcactctgtcacccaggctggagagcggtggtgccatcacggctcactgcagcctccacctcctgcagcctccacctcttgggctcaagtgatcctcctgcctcggcctcccaagctcctgggactacaggcgtgtgccactgtgcttggcagggtccattcttttcctcacactttatttattgaagagcccaggccgtttaccctgcagagtcggaatctgtacaggaggggcagccacacgagttccccggtttactctgaacttaggtggcttgagggccccagttagactgcggccaccgtttgccgggctccagatgggacgtcctttctatcagaaggctcacagtatctcctttcccgtttcttcccatgtgaacattgttgctgctgaacacctgaatatgttaatcactgggggcttgcaagatggcagtgtgctaattccatcatctagtcagttagcaggaataacttaggaccacgccctgcaccatatcagctatgtggtgatcccattcacacaggaaaggtgggacaaatgctgggggtgggccgggtgtgctgtctcacacctgtcatcccagcactttgggaggcccaggcaggcggatcacgaggtcagagattgagaccatcctggccaacacggtgaaaccccgtctctactaaaaatacaaaaaaattagccaggtgtggtggtgcatgcttgtaatcccagctacttgggaggctgaggcaggagaatcacttgaacccaggaggcggaggttgcagtgagccgagatcgcaccattgcactccagcctggcgacagagcgagactccgtctcaaaaatcaatcagtcaatcaagtgtcatcactgaatgtttgtgtgtgaacgtggggattggtcctgccccatgctccctcctgaatctcactcctgacctcagttgctgcaccttgaggtgttttcatgtgggctcttgtgtcctgaccccggcggttgtggcctctttgctgtctgggagtcaggatttttcacactcatgtcctgctccagacctggaatcagccaagtctccaagaagccctgctttcttttcctgcaagacggtatttcaagacccgccatgcggcagcgggttggtcatggttactgggttggtcgttgttactgggtgttttcgtggagatacagccatacgcacaggtgtgttcacaaatgttaattctaaaggtcaaacacccggccaggcataagggctcagcggtaatcccagcactttgggagaccaagactggtggatcacctgaggtcaggagtttaagaccagcctgagcaacagggtgaaaccccatctctactaaaaatgcgaaaattagccgggcatggtggcgcacacctatagtcccagctagtcgggagacagacacgagaattgcttgaacctgggacatggaggttgcagtgagcagagatggcgctgctgcacccctgcctgggtgacagagtgacaccctgtctcaaaaatgaatagataaataaagataaaacacctgctcctcttggtgtctccagtttggatttggcctgtgtagcctcttccttcgcctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccatgttggtggatttggcctgcatggattctgtgtggcctcttcctttccatgttggtgtccttttttccatgccaggaatcctggttctcaagggcggggttgttggcacgagcgtgatgcagactgcctttgctgcctttctcttgcccagggctgaacatggagctggaagacattgcaaagctgaagagtaagtgttgccctccccgcctccttgcagctgggtggggcctcctccttgcgaggaggtgggtgacacctcctcgacccacagtgatcctgctgcgcctggagggggccatcgatgctgttgagctgcctggagacgacagcggtgtcaccaagccagggaggtgagaggcggggagccagccccttcactgcaggcccagcctagagctagaaacgggccatggtgcagtcctgggctgtcacatcacgagtgaggcctgttttcaggcctgttttccctttttgagacctgggaggagcacctgctttgcatgatctggttgctgagatgttgagaggagcagcacacactcccacgggacagcacacagccccccacggaacggcacacacacccatggaacagcacacacactcccacgaacagcacacacactcccacgaacagcacacacactcccacggaacagcacacacacccacggaacggcacacacacccacggaacagcacacacactcccacggaacagcacacacacccacggaacggcacacactcccacggaacggcacactctcccacggaacagcacactctcccacggaacagcacacacactcccacggaacagcacacacacccacggaacggcacacactcccacggaacagcagactctcccacggaacagcacacacactcccacagacagcacacacacacccacggaacagcacactctcccacgcggggccgctgggtttcctgcagtttctcctcctccaggcctttccctggaccctggtccagtccgtcatttgagcacaggtgcctgttagaacgagaccttcttgttaggacgatgagtgtcccagccaccacctcttttggactccgggaggcctggaacgttctgaacgctccgtggggctccagtcttctccgcagccagggcagcagggtttgctgtctgtcctgcaggcagatgaggagtcagggctggggcctgtgtgggggctctcctgagcgcacagccgccgaggtggagcgtgttctgcctgagcgccgacctggtcgggggaatcccagttgcttccaggtggagccactgtcctcagcgtaatgctcaaggctctggcctggctcctcggccaccctgcaccctcagggtcccctcctgtagcttctgctgccccatcactgtcactctccaaagctttggggactctgcccagagccaccgcctcccagaagcccctgacaacctcttgacgaccccctagtgaccccatccctcccctctgacggcggcccctgctctgaggcggcttcttttcctcggtgctgttctcgtgctggccaggcctcctctccccacctggaggctcctgagggcggaggcctctcacctccaatgctggcgtcccctggagggctgaatttgtttccgagggaaggaaacttccacagttgttgccttcagttccaaagctgcagcctgatttccccctccaggctcgagcctgttttcttctcggcagctacatctttgaccagtgtcgtcccccctcaggcccgagcctgccttcttctcctcagttcccaaagctgcagtctggtccccccgccaggctcgagcctgccttcttctcctcggcagctacatctttgagctgtttgctgaagcccagatcacgtttcagaccaagggctgcatcctggactcgctggaccagatcatccagcacctggcaggacgtgagtgctggcacggggtctttggtgcgggcaaatgtggcgtagggggtgcagcaggcctccatcttggcagtcagggctcccctggccgtcacctggccgtcagcaggaacaggcccacagaacctcatcttctgatcggggcgtggaggcgttagtgccacttgccagctgccgtagagcctgtcccagttctgcagctggcggcttcgtcctacagcctcatcccattattctgcttttgagaaagagcagcccaaggccctagctggcttgtggggcctctggcttctccacaccaccccgagttctgcttctcagagttgtggggtccagaggctttgcccagaggcggtgtccccatgggctgctctggtttgagacgccgggcccagcggggtctctcctctgctgcgctcccgggtgctggggagggtggcttttgctgcttcaacccttaggcgaccatagagcctcttttcaagtcccactgacccccttggagactctgtccctgcctggcttctctcctggctgctgggaagagcaggcgaactgcccgccctgaatggatgctgcgctccaccctgggccccccattgggcaggagatggagcttggcagtcgggctgagcgggctcatgctggaagggccggggctggggtcggggcctcccctgcctgcagtgtgggtgtcagcgccctgctgccctccaggtgctggagtgttcaccaacacggccggactgcagaagctggcggacattatccaggtggggcctgctcctctgtggcatctccttccctgatggaagccgggcgggtgccttctcctgctgtattagttaactgattctagacttggggatgggagaaaggcccctacaccacctgtttctgattggcaaactctcggctcctttccagtgccctaaacccacactgggcctcctgcagggatgggggaggacgaggtctggtggcacatgcccagggtgatgctggtgagggaggacgcaaaggacagtgggggccggggagccgctcctgccctgtccgggccctcaggccaggggggacccactgctggcagccccagcagccccagctgcacgcagatgaagagctctggacacacgcggcttcctgaacagcttctccagggacagacaaatggggaccctgcaggttcccggcaggggtgtccctgggagcccatgattgggggtgcgaccctggcccccttctcattggccccgtcctgtcctgcaatgcccgtcccatgtgaggtctgcttctggctccatgcctatggcagcacctgctttccctggcgtagaggtgcttgtccggtttgtggagggcacgccccattttgggtgctctgggcacgttgcctctccggggcctcggtggcttttttagaagcagactcagaagtccctgactggggaagccaaggcacaggtggctgtgtggagccctgtgaggcctcctctgtgctgcccacgctgtacctgctggccacacgagatcatggcagggttaggcagggctgcccagcgctatgacagcttcatgagtgtccatctggcctgtggggtgcttgagctgggggaggccgcagaagaaccctgggatgcatggctggcctgtgcatgctgctgggcatggagctgcagatcccggaacaagcaggcactgccttctccttcacagacgcagctctgagcgggggcgagacctgggcagggaccaggtggggtgggcacagggtggtggggcccaggctcagccctccctccactgtggccgtctctgtggccagtgacgccacagcctgtgtcttctctgtgcggtagctggggctggaaggacagcactgccttgtcctcccaactcctccccaaaggcacggtgggcatcccaggcccagacccctctgtctgtggctcctgcctgccaagggctgctgtgctgtcccgcatggagtgtggttggctcttcaagcaggaggccgtgcacctatcaggcggacctgcttccatgtccctgatgggtcactgcaaagcacctccagcacatggccaggcgaggtagccctgcagcccagggcctggagggcaggtgtgagctggcccgggcctgtccctccctggaatacagcttcccaggctcccacttatggagaagtctcctccacactatggaactgaatcctagaatgtggcttctgaggttcctacactcgaactgaatcctggaatgcggcttccaaggcttccagctatggagaagactccacactctggaaccgaatcctggaacgcggcctcccaggcccccagctatggagaagactccacactctggaaccgaatcctggaacgcggcctcccaggcccccagctatggagaagactccacactctggaaccggatcctggaacgcggcctcccagcctcccacttaaggagaagtctccacactctggaaccggatcctggaacgtggcctcccaggcccccacttaaggagaagactccacactctggaaccgaatcctgcacactccatcggtttggaatttcctttggctgctgctctaagtagccgctggtggatgactcagcttctgccagccctcgggtgcctggaggatgagggactgcacacagtgctcacccgcgttggctcctgagcccctgcaggtgtgggcggtgcccatagggctggtgctgggttgggcctgcagccctgagtcacaggtgaccctgggggcagagtggggccagtggccccaggaagaggatgtgggatgcacagctcagctggaggcgaactccaggcagggtcaggccgtgtgctcggaagtcagggcttagctggaggcaaactctgggcagtgctggcccgtgttggggaaccagttgcccctgggcccccgtgagactgctgggtcctcatccctctctgcctgaggccggagctgccctgggctgaggcacagggggatttgtggtggtgtttttttgagaaagggtctcgctttgtcaccccggctggagtgcaggggcttgatcacagctcactgcagcctcaacctcctgggcccaagtgatcctcttgcctcagccacccgaggagctgtgaacacaggtgtgcaccaccgcactcagctaatttttaaaatttttttgtagagatgaggtcttgccatgtttcccaggctggtctcaaactcctgggctcaggcagtctgcccgccttggcctcccaaagtgctgggattacaggcaagagcttccatgcctgcccagcagaaggcttttcgaaggaagctgtttcctgaggcagactcagccctgctcatggcagccaccagcgtgggggtgaacttgttctgttacttccatccccgtgggccaaatgctttggtaaaacacaaggccctgtgtttagctgtcttgacagtgaaaatggctgggaaggaaggaaggaacggaaggaaatttctctctccttctgtgcgtacccaggcacgtgcacatgcatgcagagtacgcacacacgcacgcacgcctgcacaaatccacgcatgttgccaagtctctgtgttccagccgtggtgtctgccccccggtgttctctagttcggcttctccgcatttctgtgaatgattccggcttcttggtgttcccagcagaactccctcaagtctgcggcggggctctgacggcggtggcttggctgacatggccacattgctgagcctgttgggggctttgcgttcctgttctggccgtttttggctcgttttccaggaacggtcgtcacgcgctcctctcctagtgcaggcatcattcctttcccattgatttgcagggttctctgtaagttctgaggatcccatatacatatactctctgtaagttctgaggatcccatatacatattctctctctaagttctgaggatcccatatacatattctctctctaagttctgaggatcccatgccgacatacatattctttccttgtctcatgctggtcattttttccattttcatgacaggtttggtgaacacatgtttccttgtcagatttttgttctgagcttgtgcctcccgaccaagatgctaaaccgggtcttgtgtattctccaaactgcactgtagagtgacggagctttgtgtctgggcctccatgccttctgacgtcacctgtgggggtgtgaaaggcagactctaccttgatttttcccagcacgccacaccggtggttctgtgcgctgaccgagcggctcggcttcccccaactccactgggcacctgccacacttttcctcatgtttttgttcactgtggttttgtcgtaagtcctggtgttggcctgaaccaatttctttttgtttgtttttgagacagagttttgctcttgttgcccaggctggagtgcagtggcgcgatctcggctcactgcaagctccgcctcccgggttcacgccattctcctgcctcagcctcccaaatacctgggattataggcacctgccaccacgcctggctaattttttgtatttttagtagagacgaggtttcaccgtgttagccaggatggtctcgatctcctgacctcgtgatccgcctcccaaagtgctgggattacaggcatgagccaccgtgcccagcctgatatttttagtagaaatggggttttgccatgttggccaggctggtctcgaactcctgacctcaggtgatcctctcaccttggcctcccagagtgctgggattacgggtgtgagccaccacgcccggcctcttgttcttttgaaacctgccctgacgttttttccatagtgcatcttggagtcagcgtgtctacttcctgtaaaaatcttactgtgattttgactagaatgtgttgaattcctgttttttttttgagtcagggtctctctgttgcccaggctggagtgcagtgggaccatcacagctcactgcagcctcaacctcctgggctcaggggatcctctcagctcaacctcccaagtagctgggaccacaggcacatgccaccatgcccggctaggtttttttttttttttttttggtgaacaccctggggttgcaccatgttgcccaggctggtctcgaactcctgggttcgggcagtttgctcctctcagcctcccggagtgctgggattacaggcctgagccactgcactaggccatgttgaatttctagattaatttggggccctcaggggcacagagaggagggctgggccagttggcgggaggagaggcccctcgggctgccgcattttcagtgcatggagatggcctatgttgggggaacacagagctcaccgggggtccctgcagggaggagaaagggtcaggcaggtgccagctcctgtccattggcctggggctgcatgatggcaggggccggtgaaccgatgacccctgggtgtcctgtgaccttctgtgtatgcggctgatgctgcagaaagtcgggtggcctcaggctcctgacggggctgcacttcctctgcctttcagattgtgttcagtgtggacccctccgagggcagccctggttccccagcagggctgggggccttacagtcctataaggtaggggccacctccaggaggcaggtggagggcagcccttgttccccggcagggctgggggccttacagtcctataaggtgggggccacctccaggaggcaggtggggctgggggtcttctggtcctaaaaggtaaggggctgcccccaggacatgggcggggcctccacactcctggtcctgtcccctccaggtgcacatccatcctgatgctggtcaccggaggacggctcagcggtctgatgcctggagcaccactgcagccagaaagcgaggtacagacctgggcccacacgctccccgcccgcccgggtgcagtgcccggcaccaccatgccacaggctaggcacatgcccagccgtggatctcctgcccccatgggcctggccaccttctccatatccaggccaatccagagcattctcctcactgtccctctgaagattggagttactgagagacgtaggagatggcctgatggcaccgtgacctgcccagagtcacctggttggtggtggcagagccacagcccagccaggcctccctgctgggacacgctcgtttatgccgaggccgtcagcacagagcctccacagtgaggcacggctctgcctgctgcctccacgcagcgcctggccgggccaagcctcagggtcacatctgaagggggcccggctggccctgttgtccgaagcccctggtgcgctcagccccgaggccccacgtgccttcttggcttcctgtgctccgtggcgtcttcgagtcggtgctgccggggacgctgtgtggatggggtctgtgagtgtgccctcggctccgtgtccggagccctgtggttcttggggtgtatctggccccacccccactgcgtggtgtccagggtggggcttcacggctgcagctgcgggagctgctgcccctgccttgtgctccagtggggccttgcctctgggcttggttcgtccctctctggaacattctttctcagctgctgtccgacccatggtggcatgacgtggccctggctgaagcagcccttgtgcggttgctgtggttgggtctgcctggccgagccggaagggaagggctgggagggcgtcagggtggcgtggcttgacccccgctcggtgatggtcctgcagcaaggcctctcccagcaggaagcgtccatcccggggggaggccggcgcccctcacgcagttggggttgcgggaggcagtgcgtgcctgaggcagccggtgcacagattccaagggcctggaatctgtttgttccattgacctctgatgtcacttgacttctcagaagcagccactccctgcactgggcgtttgtaggaaatgagctcctggaggagggggtggggaagttcccccattgcagggcacactcagccccaggaaggaaacgtgcctcgtccctgctgactccgaatcgcagtcagagtcgttctgcttgtgccgtgttgaattcccggcatccggcatccagactcagcctcctccccaggccacggccgccgtggccagtcggtcaagcccttctaggaacttcctttgagctggcgcccttgttcactgctgacgccactcagaggcttgtgcacgtgtcctgcttccaggcagagctgggaactcgcaccccgtcttctgcacgcggccgtggaatgtcgggatgccggcgcttccttcccgtgtgctcttggcggggtgggcttcttgccctgagccgcatgtcacagtttctgcagaagtttagggttggagtgggctgacctctctgcaggtgtccccagcctctgcctggggtctgcctcctactcccaggaccccctgtcccccagaggggccccaagctggcaggctcacactcagggcagcctcctttgttctgacttctgcacagtgggcctgggtggctgcccgcggctcgcttgcttgatgccagtgggtggagagggtgatgggcagagaggcaggtggtcaggcccccagtcccgtcctcacactctgtgccctctgccgccccccgccccacagggaaggtgctgagctactggtgcttcagtcccggccacagcatgcacgagctggtccgccagggcgtccgctccctcatccttaccagcggcacgctggccccggtgtcctcctttgctctggagatgcagatgtacgggccacccctgccagggcctgagcaccggtgacacctctgacatcagcggggtggaagtggtgggggtccccatgagccgggtgctgggggtctcgggcctcgagggctaaaggggtgctggtgcacttccccactgtctgctccctctggccacgctcagccctttcccagtctgcctggagaacccacacatcatcgacaagcaccagatctgggtgggggtcgtccccagaggccccgatggagcccagttgagctccgcgtttgacagacggtgagggcctgtccctgggccctgctggggtgggaggtgggggagcactgaggcctgaggtcctgagcagtggcctctccggctctaggttttccgaggagtgcttatcctccctggggaaggctctgggtgagtgccctgaatgccccagctgtgcccatcctggatcctggacccctgctcccaagagctggtagggaaccctgcagacatcctgcccctgccttgaccccggcccctgcacttccaggcaacatcgcccgcgtggtgccctatgggctcctgatcttcttcccttcctatcctgtcatggagaagagcctggagttctggcgggtgcgtctcccctgtgttctgggcggggtgggtgagggcagggctggagcatgaagcaggcagtggtcacagctcctgcttgccctcatcggatcggcggcgtgaccagggctgccgtgtccctgcctcttcctcccacaggcccgcgacttggccaggaagatggaggcgctgaagccgctgtttgtggagcccaggagcaaaggcagcttctccgaggtcggcacttggccggggctctgggcctgctgccccctcgtgcctcccctgcctctcacagcttccccaaggctgaccactggccctgaccatgggctccggcggctcccgctgcctcttcagggctcctgcgtttccttcctggccctgagtgttgcctcttatcttacaaagcccccagcaccgggtgggtgtggtaacagtggccctcctgtctgagtagccctagtcggccaccctggccctggggttccccgtgttttctgggaagcactgagcaggcgtggggtcagcctgggatccgtgccaggaagaagcttccagaacccgattggccttcctggctaggacgatccttcatcttggagcatgagacctgggtctccctcatgggggaggaaggggctgggggggggctccaggctcagcctcaccaactttccttccagaccatcagtgcttactatgcaagggttgccgcccctgggtccaccggcgccaccttcctggcggtctgccggggcaaggtgagctctccagggccctctgccctgacctggttgcctgttccctggtgggtgcttatggctccccagcagactctgggccctgggggctgcccggtcccctccttgggtcccacgagagcgactgctggccctgctgggagcgtgtcctgctctgggcctgggcaggcaggatgggagtttcctggccacaagagttggaggtggcgtctgggagctgtggaccccaagtggggtcctgacccacagatggagcttcctcccacccctggttggggacggagcctcggggaaggtggctgggctgggtgtgggcaccagggagaggagcccccacggccccaggcagctccctggtgtgtcccctaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccaggtgagttacagcagggtggggctggggtaaggcggtctggtgactgagcccccgccccgtggccaagggagcccccgtgaccgagccgcctcgccccacagttcctctctgggcaggagtggtaccggcagcaggcgtccagggctgtgaaccaggccatcgggcgagtgatccggcaccgccaggactacggagctgtcttcctctgtgaccacaggtgcgtgcagtccggtggcaggcgcggcgccaggggacacgcccacaccccactgggcccctggactctccttccccacatgaggccccgtctcctccagagcctctccggctactcggggtcagcgtggggcccctgcagcagatgagggtcttcacttcggtgaactgaacccttgaagcggctgtgggcagggcagcagggctatggccaccccccaggttcgcctttgccgacgcaagagcccaactgccctcctgggtgcgtccccacgtcagggtgtatgacaactttggccatgtcatccgagacgtggcccagttcttccgtgttgccgagcgaactgtgagttcctgcccagggaggggatgagggtgttgtccccagaggagccagaaatgggtccacccacccccatggttctgcagatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcaggtgcggacgggcagcgctgggtgggcggtgtgggggtggcggagcgggcggcgtggggcgggcagcaccaggcgcccagggcggaggcgactcacctggctttgtgcgcttcccctcccacctccaaaggctgcctctccctcctagggcagggcccccacgggctgcaaccctcccctacaggcagagaacgccccaggcaaggatgccccccgaggctgagactccccccaatagcagggaggacacccacaggcaggaccccaagtgctgggactctcccccaagaggggctttgccacaggcagggaccccagctggggccccccgtgggcttcactgcgcactcgggtgcccctgcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggtacagttccagggccttgggatggacacagaccctctgtctcctgaggccaacccgaccccgcccatctggcctcaggcacctccccacacacccctgtaaatcccctgcctggcaggcaggcgggcaagcgggcgggggatcccagctgcctggctgtctgtgggtcctccaccccacctcacccacaggctgctggctcccaggtggtgcatgccctggccctccgcgggtgccccccacatcactttggttctctggcgggtcagcttggctcagtgcactcaaggtcgggtgcccctgccactggctgcgcttgaggctggcctttctccaggaatgtgctgcgggtggaacccaggttccttcttccttggggccttttgccccagaagcccataattcctcaggccaacccgaaattttctccctgcttcctgctgggagccattcccctcttcctgcccatccctgcccttcaggcccctggagtgagctccaggtgcaggcaccaggcacctgtgtccccttcctgccagcccctcgctgtggtcggactgtcttccctggacctgctcttacaagtcaccacctgcgagcctcatgagccgctggtgtgacttggacaggaccaagttgtggcactgtcaccggggtgtgctgtgcccccctcccccgacctccatcttggctcagggctccttgggaccatcttccctgtgcgtccaggtgctttgggaccccagagtgtgtggttggggtctgtgtgtggttgtgagctgtgtcctcctcaggcccacagctgctccaccctgtccctcctgtctgagaagaggccggcagaagaaccgcgaggagggaggaagaagatccggctggtcagccacccggtgcgtgagctgtccctgcacctgtgccgaccaccatagacacgcatgggaacgcagccgtgggtgcccccagccacggctggtcccgatgggaccagggaatccacccccaggagctgatgtccagggcagctgtgatgctgacggccaggggctcaagtgtgtggtttcttctgcagggggctcatgagtcccagctggaatcaggccccacccttgggcaggtttggcatggggcctgcagcactgggcttggccctggcatttccctcaagtgtggatgcacacctgcctcatgtgagggacacagcccattcctagccttggatcaaagaacggagttatagccggagccaggaagccccctgcctgctggaaaaccccaagtgtggcggcctttgtccatgtcccttggcttctgggaagaactgggtggtgcccaggcagggctggtgccatcaggaagtgggtggctgctgaggggcctgggctggcgagggcctgggtggggagtgcctgggccgcccctgccttggtttccacgtttccgtgttggtctggggtgtgtagagagatgggcactgctcatccggaagcccctccttgtgcgctgccatcctgggagcctcagccgcatccgctgtggggcagggggcttgagggaggaggagagagacgggccatgcaggacccctggcttgaggcagagccaatctaccctttgcccattcactgctctcagttccctgccagcctctcactgtgtgacctcagacgggcccagccccacagctttcttcccgcagcccctccctatgtccatccagccagccagtttctcaggcagcagccccacctcggcagtcactgtcccagggaacgctcaatgttccaaggaaggctctgcagccccagggaccagatgatgaggctggccctgatggagcctcgggcctgtgtcctgcaggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggtgccctggcttgcagaggccacccaccctgagggcagtgctgccgccgcgtgtggggtgggggccatctgggtccaaggtggtctctgttctctagagaaaaaggggcagatggggacagacgccccttcctctacaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggtaaatggggccccaggtgggaccctcagactcctgcgtggaaggcagtgtgggccagagtcctgggctgcttggggtgggcatcctcgggccctgcttggccccgcctctctgttcccctatgggagtgatgggggcctccacctccaccaccagcaccagcagcaccacctccacctccacctccacctccacctccaccaccacctccacctccaccaccacctcctccaccaccaccacctccaccaccaccaccaccaccaccacctccacctccaccacctccacctccaccaccaccacctccacctccaccaccaccacctccacctccaccaccacctccaccaccaccacctccacctccaccaccacctccaccaccaccaccaccaccaccaccaccaccaccaccacctccaccaccaccacctgcaccaccacctccacctccaccaccaccaccacctccacctccaccagcagcagcatcacttgttggggagaccctgtgcaactccatgcacagccctgtccctgccatagccccgacccctaagcacagccctgtccaactgccacacgtcccctgcctcccatgcatggtcctggggggtcaactgcacacgccagggtcctagggtcctagacccctgtcctccctgtttctgcctctgtttggggtggagtccaagtctccagaggcggaagcatctgtgttcgtgtgttaatgaacagcccctacagagttcccctagttcacccaggggggaacctagcctgttgggacgaccccagatcccttctgggcttggtactcactgggatatcctcatgcctgcacccagcctacggctctgagctcctgagtggggctttggcctgcccgccactgttccagcccccatccagcaggctggtgtctcctctgatgcccccagcacccaggcgtgtacctgcctgggttttcccgccctggtctgaggtgggtgaggcctggcctccctagccagccctgcccccccaccccagggaactttccagatgctctcgaccagctttgtggctctacatctcttcatcaggaagaacggcgccggatcccaagctgaccgtgtccacggctgcagcccagcagctggacccccaagagcacctgaaccagggcaggccccacctgtcgcccaggccacccccaacaggtagctgactcctgaaccgtgtgcagcctacgacttggtgggtccctcagtggcttcacgaggctaactcttgagtgtggccggggctgcccctgtggggagccatctcatggtggggactgctcccggttctgcaccccgcagttgtcctgagcagctctccaggagttcctggaggaagggcgggcagggcggtgggactctcagtcctccaccccagcgccactctgagccatgctactcccacaccaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacagcaagtggccctggcgtggggaacagccggtggggtggggggcaggggacaaaatgggggctgtgccgggtctgattgaagctccccgcagggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggtagggcacctgcctggctgctcctggcagcgccccaaccgcacgcagccctgggagtgagcagcaaagccccaggcccccctcagactcaagtctctgtctccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggttggtgcctggccactacagttcctgctgggtgtagccccaggtgatgggctgagggggaaagggcaggcccttgtcctggtggcaacgcctggcagacgtgtgcagtgggccggttgtctcacaggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaataaagctgctggcagtgcccaggacggtg

SEQ ID NO: 2 - DNA, 37214 bp, Macaca fascicularis
  misc_difference 4525..4624: note = misc_feature - n is a, c, g, or t
  misc_difference 4914..5013: note = misc_feature - n is a, c, g, or t
  misc_difference 9472..9571: note = misc_feature - n is a, c, g, or t
  misc_difference 16613..17049: note = misc_feature - n is a, c, g, or t
  misc_difference 19812..19911: note = misc_feature - n is a, c, g, or t
  misc_difference 35806..36357: note = misc_feature - n is a, c, g, or t
  source 1..37214: mol_type = other DNA
atgcccaagatagtcctgaatggtgtgaccatagacttcccattccagccctacaaatgccaacaggagtacatgaccaaggtcctggaatgcctgcagcaggtagagcacaggctgggaggaaaggactgcaggcaggtggagctggggctgggtgtgcgtccctctcccgacccattccagccaggcccctccagaggcagcctctgtcataaaaaggctggtgttccaggtggggtcagagagaggattgacaagtaaaaacatcgtccttgagagtgacagacttagtgccttgggggcctctgaggagcggctggtattcacaggtcaggaggaagcatttccatcagaggggcagccgggggccagcctcactttgaaggtctttgaacctttggggtgcagggaggtggcagcggcgcaggttgccttctcctgggttccttgaggtgccctcttgtacccggctcacacccctcccctccccgagtttcccgctcaggttcccatctgagagctcgtatgtaggacatcagataggacagcgtaagtgtttggatctggaaacacagaacagtttcctattttgagacttgatgcctaattagtcatcttactatttaggctgaaaaatactgttgcatttcgggtaacgttctgcaaatggtctgctgatggcagctggagttgcttcacgccctttagggcaagagtggaaagtgtcctgcggacttatccatggtcccgtggggctctcgccacctgacagcggcctctgcatctgcgaagagctgcccactggctgctgaggcttgtctcagggtggtttgtgtggccttgcctcttcctggcttccccgtaacccttgcccccaactctgttcagaaggtgaacggcattctggagagccccacgggcacggggaagacgctgtgcctgctgtgtaccacgctggcctggcgagaacacctccgagacaccatctctgcccgcaagattgccgagagggttcaaggggagcttttcccgaatcaggccttgtcgtcctggggcaacgctgctgctgctgctgaagatcccataggtgaccctagttcccaggccttccctgacctcctgtggggatggctggcaagggatggcgctgagggtagggtgggcccatggggacttctgccagcagagctcaaggagaattttgtagctgccacataatttctcgccatcgtgggtataaacctagggttgggcttttttgctgaattagggcgtggcaggtgtacacttcgcccgtttgtgataaacgagtctctggggtgtcagattcttggctgtctgcagggctgagttagccgaatgccacccgcctttaacacgtgagaaccatgacctctgtgcttgcttgtgtctgggcagcttgctacacggatatcccaaagatcatttacgcctccaggacccactcgcaactcacgcaggtcatcaacgagcttcggaacacctcctaccggtgggtcagacgagttcacgcctgtcttggggtcctcgagagaaccagcttgacgtggtgctgggtccacagaccacgctgtgctgtagtggagggggcggtctttccagacgctccccagaagtatgcagtgtgctggtgcccaggggtggggcggggcccgggctgtccctagtgcccattacttgtgaggaggcagctttgcatttgtgtgctgaccttgggctggcgtcctgagctcctttcaggtgctgttgtggcagctgtgtggcaggtcagggccggcccccagtgcagctttgcacatgaagtgggaggaggccctgccgcttgtcagagccctgcagagtcttggtgttctgtcgggttcctgtggaggggccgatggcggggtgctgtggaagctgtcgaatctcgtccctctgtccagttcccccgcttgtctccttgctccctcctactcccgggccacgatcctatgccggggccactctcctgcacccggaccacgctcctacgcccaggccactctcctacgcctggaccacattgcagttatgctgacttcctctggctgccagtgctcctgtgtgtctccatacagctcacgctgcgggaccacgctgtggctgttggaggcagctcctcctccacccatagtgctctctccctccaggcctaaggtgtgtgtgctgggctcccgggagcagctgtgcatccatccagaggtgaagaaacaagagagtaaccacatgcaggtgggctcctggctcccgctccggctcagggttccgtgaggcgagtgctgctgggtatccagagtcccaggctgtgctcctgctgggctggggttcgaagttcactgggggactgcaggcgagggcctggtgggggtggggactggctcgggtcctttcttggccatactccagccgcgcactctgcccttcctcccgcagatccacttgtgccgcaagaaggtggcaagtcgctcctgtcatttctacaacaacgtggaaggtacaggcagctcggtgggaccaggcttgggttggagtgtgtgcagcctctcagggcgtcagcctggttgtgcttgccgggtggggcggccagtgcagccgtgtacctgggcactgtcttctgactcgggaccacccgtgttagtcttctgtgtggaagagctcacccagtggtctgagacagccagccggccggactgcctgtggctggtgcctggggccttggattttgggaaggctccctccatttccggacgagaagggctccctgcacctgactactggtgcatacagcagcggttttgctgaacacctgctttgtcttcggggagcctgaggttttgttgcttgcccagcagcgggtgctcctgtgaccggcccttgtaccatcttgggagggtgtcctggaagccgtgtctggcctcccccgaccttgccgcatgtgtctttgtcctgtgctgacgttgcagagaaaaattacagccccgagcgtgactccaggctgagtcctgtgggtccgacacgggaggccttggggcctcttcaggagacgggatgagtgagtgatgggagcagagccagggcacctcgccctgtgactgcaggtggccacagcctgtgagggccaggggtgcttctccacccacgtggctgcccctcgggtatgtcaggggcttctggggctcttcacgaggtctcagagacagtagcagggtgtgcccccgtcggctgcccttacagtttctgtgacctgagggtggcatctgtgcggttggcatggcctgagcttctgtgggatcagagttccctttgtttcctgcctcagttggggctcaagcctcaggtgacgtggccctggagcacctggaaggcgtcggcggtcctgcgggctgctgtctgcactcgtgtttgctgagtgctcagtatgccaggactgaggaccctgaagctgctgttatatttaggacggttctcccctggcagagacggagccgggtggtcccgcctgacccaccaccaggcgtttctgggccctggagggacagggtgggcggaacatgggcctgcagggagacctccacttactggaggctcgtgctgtgttgctggaggcatcttctgtgttgcttcttgtttgctgtgtcttttgttctggtggcaccaaggacctccagtcaccttgatgtgtggttgtccaggcctttttggtggtcctgagaaggggtctgaggggctctgcctctgccgccaggttccctgatggaagctgcccactcgtcctggtggtgcccacaggacgtgactctggcacaggagggcagatgtgctgttggagattctcagcccgtcgtcgtcatttccaaagtcctcacccgcatttctgggacaggacaggagtgcctgctgggtgtccccggtcccatgcagcgggggtccttgggatagtgtggaacgctgagtgtgggcctgtccggcggtggtcctggacaagggtagcacccctgtggccgccgggcctggtgcctggtgcctagcggggaggccgctgaccgcctcctgtgctgcttccatccgtgtcaggcctctgggtgttgggcccccatctgtctccccccaggcctgtgggatcagtccaagaagactcaatccagccccagcctgtcccccttggcttgggctctcactgcccgacctggcaggaggttgcctagcggtgaacctttgcatcctgtctgccccctggacaggctctgagggggtgtctgcaacacctgtgccagcctgggcatctttttttttttttttttcttttgagacgaaatctcgctctgccgcccaggctggagtgcagtggcgtgatctcagctcactgcaagctccacctcccgggttcacgccattctcctgcctcagcctcccgagtagttgggactacaggtgcccgccaccatgcccggctaattttttgtattttttttctttttttttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgagacggagtctcgctatgtcgcccagggtggagtgcagtggccggatctcagctcactgcaagctccgcctcccgggtttttacgccattctcctgcctcagcctcccgagtagccgggactacaggcgcccgccacctcgcccggctagttttttgtattttttagtagagacggggtttcactgtgttagccaggatggtctcgaactcctgacctcgtgatccgcccgtctcggcctcccaaagtgctgggattacaggcttgagccaccgcgcccggccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgtatatttttttagtagagatggggtttcaccgtgttagccaggatggtctcgatgatctgaccgcgtgatccgcccacctcggcctcccaaagtgctgggattgcaggcttgagccaccgtgcccggccagcctgggcatctttagagtgggctgcagctcctggaggggtctgagaggaaggaaggcaggtattttgtgaatgaagagacagctagagagctggcacccttcctggcctgcgtcctgtgaggactctggttggggacagcaagcttggggttagcctggggcagagcctctgggaggcccctgccccttgtcccccttccccttgcagctcctgtcctcgccccgccctcagctctctgccagggaaggtttggcaagtgccgctgtgcggcagtgcgtgctgattggctggtcgttgctatggtgccgctcagggatatgcttttcctcccctgccttccctgctatccctgggagcatctggagttgagtcatcgctggtgtgtgtgtgtgtgtgtgagtgtgtgtgtgagtgtgtgagtgtgtgtgtgcgtgtgtgtgtgtgtgtgtgagtgtgtgagtgtgtgtgtgtgagtgtgtgtgtgagtgtgtgtgtgtgtgagtgtgtgtgtgtgtgtgtgtgtgtgtgtgtgtgagtgtgtgtgtgagtgtgtgtgtgtgtgtgtgtgtgtgagtgtgtgtgtgtgtgtgtgtgtcagtgtgtgtgtgtgtgtgtgtgtgtgtgtgagtgtgtgtgtgtgtgtgtgtgtgtgagtgtgtgtgtgagtgtgtgtgtgagtgtgtgtgtgagtgtgtgtgagtgtgtgtgagtgtgtgtgtgagtgtgtgtgagagtgtgtgtgagagtgtgtgtgagtgtgtgtgtgtgagtgtgtgtgtgagtgtgtgtgtgagagtgtgtgtgtgagtgtgtgtgtgtgtgtgagagtgtgtgtgtgtgagtgtgtgtgtgtgagtgtgtgtgtgtgtgtttgtgcccgtgtgcgcctctggcctctgcagctgagtcctggctctcagggggcctggcacctcccggggatgggcacaaagcagccacgatgcagccgggagctggggaaggccccattgccccacacggctgccctaagactctggggtgcttcttggaagaggtccttgcttctgtctgtgtttaagcagctccctaaggagttcttgtggttccaggttgggggcctgtgctgtggaggcgagggaggggcaggacccccaactccctgaagccttttctggcctggtcctctcagccagcataggcagggccctttcccagggctgccatgctgcagaggggagcgggccactatgtagcctaggaaaacctgacgttccttccgctggcagttctgggcctgttgtggttggcagggaagctgagccacggtgctcatcacaggggcacctccaggatttttgggagatgcctccgtgggctggggcgatacgctgaggagctgttcttccctgccctgaggagggctgagtgtagccgtcacccctgtcctgtcttgggctgtctcagggaggatgcatagaaccctcggaaccctgctggcccttgtctgatccaccctcaacctcaggccttctaggggcacaggagggctccgtcgggatcactgcgggtgggggcctcccttggacacctgagaccctcaatgggtgctttgtggcacgttcatgattggggcggggggcacccagccctgcctgccgtgtaggagctgttctgtcctgggcgtccctctgtggttgggacccggagggtgtgtgtttaccccgcctcacacctgcagagaaaggcctggagcaggagttggccagccccatcctggacatcgaggacttggtcaagagcggaagcaagcacaggtgagacccctcagtgaggccacgaccactgtccttccatggcccagctctcctgtgacctgtggaggcccagatatgtttcttcccttttctttgttcctttctaagtcgtgaaactaaccaccgtttagtgtgagaaagttgaagcagctctgaggaacgtagagtaaaaagcgaaaccgcctccctcttccctggccgtcagccaccccaggagccaccgtggagcggggacagagccctgcaggctcgtgtctgtgtggaagcacgcacggttttgtctgcacagactgttctgcagtttgccgttttcactcagcgttgtgggtagcttcgcatgctggtgctggtcgtccggcttcgttcttctgaggacagcaaatatgtcctttgtccacctcttttatagtttcagagattcaagttataataaagcttttcttatattgagggggagagctcctgcccctctttttttttttagacagagtctcgctctgctgtccaggctggagtgcagcgtcgtgatcttggctcactgcagtctcaacctcccaggctcaagtgattctctcagctcagcctcccaagtagctgggactgcaggcatgcaccaccacgcctggttaatttttgtattttttgtacggacaaggtctcactctgttgctcagtccagtcttgaactcctgagctcaagagttccacccgcctcggcctcccaaagttctgggattccaggcgtgagcaccacgcccgacccagctctgctttgttttagtggttctgtttgtgtttgtttctgatgacctctatccaggaatagggttggtttttcttttccatggagtttttaaagagatgatgatttacatggtttaaaactcatgtggactccccgtccatcccagacagcggcttcccacctggaccccatgctcaggattagggctccttccgggtcagaagatgtggcggccccggcctcgggtcaccgtcccctgcacggggtgctcacagcatcttcccctctcctcgttcccagggtgtgcccttactacctgtcccggaacctgaagcagcaagccgacatcgtcttcatgccgtacaattacttgttggatgccaaggtgggggctcagtctgcgtcgctgacaactcctgatgtccaggggggttcctgggcttgggaaaggccgtccgagcctttgctgctgcagggacttagagcagcaggcctgggtgggagagctcacctgtcactgggcaggggctcaacctggccagacacacttgtgagcacccccaggccctgggtcagttgttctgagcaatctgggagtgggcaggctggtgggagtgaggagaggcctccaggctgtggtccacaggccagcgcccactcttgatcctgacagctcaggttctctccttcacgtccgatcacaggaagcaaagcactgagaacacgggaagcccactcactcccctcctcccagccccacactcactcccccccatgcccggccccacactcacttccctcctcgtagcctgtactcacccacatgctgttgtgccccacactttttatttagtgcaggtggcatctcgccgcgggtcagtgacttgtgttcacgtggcgatgacgtggtgacgcttccagatccctccattggtttgctcattctcagggtgtatatttatcaagagcccatcgtgctgggggctgttctaggcatagcaagactggcttcattcatgtggagctttgattctagcggtggggacaggtagacagcagaagagtaagcccgtgagccgatgagactgaagagaagtagagcagagcgaggaggtggagacagagccgagtgggcagggcccaagtgcgatgtcggcaggagatggggaatgctgatgtgtctgaggggagcccgagctggtgcagcagagcaagggaggaggcggggggccgactcctgggcagcgattgtagaaccccacggagagctggtgaggtttgccgtggttgcaggtgactcggtgctttgagccgtggctgcccctgggaaccacctggagagcttctaacccaaccaggcccttccctgggacaattctgtcacagctggtaagctaagtccaacactttcacggaaacacagaagatctaaaacggcacgatgaccgtgaagaacagagctggaggactcacctcactggtttcaagactcctctaaaactgcaggagtggaggtggagacggctcagctcaggcacaagcctgcaggccatggagaaggcagcaagctccagctgacccacacgcttgcggtcattgttttttttttttagttggaatctccctctgtcaccccagttggactgcagtggcaccatctcggctcactgcagcccccacctcctgggttcaagcgattctcccacatcagcctcccgagtcactgggattacaggcgtgcgccaccacgccctgcccgtgatgattgttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgagacagagtctcgctgtgtcgcccagggtggagtgcagtggccggatctcagctcactgcaagctctgcctcccgggtttttacgccattctcctgcctcagcctcccgagtggccgggactacaggcacccaccacctcgcccggctagttttttgtattttttagtagagacggggtttcaccgtgttagccaggatggtctcgaactcctgacctcgtgatccgcccgtctcggcctcccaaagtgctgggattacaggcttgagccaccgcgcccggccgatgattgttttttgacaaatgcgccaatgtaattgagagaggaaatgaaggttgatttttggttttctgaaaaagtggtgctaagaacagctggatatctgtttggaaaacagcgaatcttaactcttgctttactctatataaacctaaatgtaaaagctaaactaaaggttatagaaagaaacatgggggggtctttgcaactctggggtaggcagagattccttagtgtggatacataaggcactagccgtgaagaaaaacataaaatttggatttcaccaaaatttaaagcttcaacacttgagaagacttgagaaaattaaagagcagttatggaaagggagaaaatgcttctgtctttcagaggacttaaaaaacatttttcagccctcctcatattttaaaggacctttgaccagagtatgtaaaattctctcataactaagcaaaaaacccatttaaccagtgggaaaggatctgcacagacgtttcaccaagatggacggagtggccagttgggcacatggaaggacgcccagcatctccagtcacaggagaaggcagattaaagccacggggagccggcactacggtcccgctagcatgctgaaattcagaagcctgagtgtggcacgaggaggtggaacagctggatctcatccattgctcctaagctgtgaagctgtgtagccactccacaaacacgtgtggcgaacagccgaaccgggagaagggaagacgtgttcaaagatttgtgtgtggccaggctcagtggctcacacctgtaatcccagaactttaggggccaaggctcagggatcgcttacgcccaggagtctgagaccagcctgggcaacatagggagaccccatctcaaaaaaaaagaaaaaagaaaaaaaaagacttaaatgtgcaagtttaccagagctttgtttaagttgccagaactgggaagccgcccacgtgagtccatccactggtgaacggacaaaccgtggttcgcaaaccccaacagcagccgcgggcatgggctgtggtcacacagcagcagggagccagtgagtctcagatgtgctaacccagaggccaactgaggaggacctactgttttttgttttttgatttttgttttgagacagagtcttgctctgtggtgcaggctggagtgcagtggtgtggtctcggctcactgcagcctctgcctctcgggttcaagcagttctcctgcctcagccttccgagtatctgggactacaggcacccgccaccacacccggctaatttttgtatttttagtagagacggcatttcgccatgttggccaggctggtttcaaactcctgaccttgtgatccactcaccttggcctcccaaagtgctgggattacaggcgtgagccaccgcacccagctggacctactgttttattccgtttatatgacgctgtattaatagaagaggcaggggtagagctggtggttatgtggtgcacttaactgccagaactcagtacccttaaaatgggcatcttaatgtgtgaaaacttaaaagtaccaaagctggaccccaccccagcttgctcccatgacactctgtggtgggacctgggagttggggtttttttttttttttttcagatggagtctcactctgttgcccaggctggagtgcagtggtgcaatcttggctcactgcaacctctgcctcccagattaaagcgattctcctgcctcagccttctgagtagctggggttacaggcgcacaccaccacgcccggctaatttttgtgtttttagtagagatggggtttcaccatgttggtcaggctggtctcgaactcctgacctcatgatccacccgcctcggcctcccacagtgctgggattacaggtgggagccaccgcgcccggcctggagttgggtttgtaaatctccctgagtggggccggggcagagaactgctgggtctgggtcttcctggctcctgtggtctgtggctggctgactgcggtggccgggggctcccaggacgtcgtggccgtccgtcttgctgagcgtggcgcgtgcctttccatgctgtgcgtgagcgtctcccggcgtggcgagctgctgcttcgggtggggcggtgttgccaggttgccatccagatctggcctctcctctccacgtcgccggcggtgtttgtcatcggcacttgcgatgttcgatgccggccacatgtgccttggtttccctccttcctgcccctgtgagctccagcaccgtgtcccttctcttcctcctgtagagccgcagagcacacagcattgacctgaaggggacggtcgtgatctttgacgaagctcacaatgtggtgagtctccactgcctcctaaacgcctcctgtttcttcaagagcagcgcaaacctttctggaggggctctggccaaacccttgcagccttcggtgcccaggactgaggactgagcaccccaggagcttctgcaccccttcccactctgatccgatgcctctgctggggctggagactggccagctgggccagggacctgcccctcaggcgcagggcccccacaggccgctccccaggccttccctctagccagctaggggtcagcctgggccaggggtgtctcctctgccctcggcttctgtcaccagggcagcagcaggcgtgtggtctcgcctgcagtgtctctgcccttccggccacatggcctgaggctgaggcagaattgcttgaaccctggaggtggaggctgcggtgagccaggatcacaccgctgcactccagcctgggtgacagagcgggattctgtgtcaaaaaaaaaaaaaatgttgactgggcgcagtaggtcatgcctataatcccagcactttgggaggccgaggcgggcggatcatgaagtcaagagatcaagactatcctggccaacatagtgaaaccccatttctactaaaaatacaaaaatattagctgggcgtggtggtgggtgcctatagtcccagctactcaggaggctgaggcaggagaattgctcgaacccgggaggtggaggttgtagtgagccaagatcacactattgcactctagcctggtgacagcgagactccgtctcaaaaaaaaaaaaaaaaaaaaaattggcaactccattgaaataattgtttgtttgtgttttgagacagggtctcattctgtcgtccaggctggagttcagtggtgtgatcttggtccactgcaacctccacctcctgggctcaagccatcctcctacttcatcctcccaagtaaccaggactacaggtgcacaccaccacgcctggctaatttttgtattttttgtagagatggggttttgcatcattgcccaggctggttttgaactcctgagctcaagtgatctgtccacctcagcctcccaaagtgctgggatcacaggtgtgagccgctgcgcctggcctgaaataatcgtttctcaatattggtgtgggccagacagtcgtgtttggacctgcttgtggcctggccttagagaccccagggcgtggctttggggacttggctgtcagcctcctgtgccttctgcacccaaccccattcctgctttctggaacccccgatcctatcctgctctgtggtgatccgggtgatttgggctcccgatcctatcctgctctgtggtgatccgtgtgattcgggctcctgatcctatcctgctcggtggtgatcccggtgattcgggcccccgatcctatcctgctctgtggtgatccgggtgattcgggctcctgatcctatcccgctctgtggtgatctgggtgattcaggctcccgatcctgtcctgctctgtggtgatccgggtgattcaggctcctgatcctatcctgctctgtggtgatcccggtgattcgggcccccaatcctatcctgctctgtggtgatcccggtgattccggctcccgatcctgtcctgctgtgtggtgatcccggtgattcgggcccccaatcctatcccgctctgtggtggtccgggtgatttgggctcctgatcctgtcctgctctgtggtgatccgtgtgattcaggctcctgatcctgtcctgctcggtggtgatcccagtgattcgggcccccgatcctatcctgctctgtggtgatccgtgtgattcgggctcctgatcctatcctgctctgtggcgatcccggtgatttgggctcccgatcctatcctgctctgtggtgatctgggtgtgctcggactccaggagaagatgtgtgaagaatcagcgtcctttgacctgaccccccatgacctggcttcaggactggacgtcatagaccaggtgctggaggagcagaccaagactgtgcagctgggcgagccccacccggagttcagcacagactcccccagcccaggtgcgttcatagccaggctgctcggtcctgaggcctgcgctgctgcagggggcagcctgccctgtggctgtgtgtggtcggcctgggcactgtctgttcaggctggcactgcagggcacccacttctctcagaggcttctcgggtgtgaattgtttggggtcctgtaggctgtggaagggctggtatcgttcagtagttctggtattttccgaagacctatgtcttctcccagccagtgtcaacttggcctctactgtgtaaaattagagaactatactttgtgaagctgagatgggagcatcgcttgaggccaggagttcgagaccagcctgggcaacatagcgaaacctcgcctctgccaaaaaattagccaggtgtggtggtatggtcctgtggtcctgtggtcagagcttttctggaggctgaagtgggaggagctcttgagcccgggaggcagagcttgccccagatggctccactgtactccagcctgggtgacagagtgaggctgtctccaaaaaaaaaaaaaaaaaaagtaaaactaactatattcactgtaagggcattttgcgtgtttaaatgacccacacatcgggcctgcatcacttgctgtgcccgtagattccttcccagtgttcgtccagaggcgtatttccacacagcgctagtcacggcgtacgtggaaaacgcggaaacccccatggatgtggtcggttggtctgtactgtctgtgttttcttcctttctttttttttttgagatggagttccactcttgttgcccaggctggagtgcaatggtgcgatcttggctcaccgcaacttcctcctcccaggttcaagtgattctcctgcttcagcctcccaagtacctgggactacaggcgcgcgccaccacgcccagctaattttgtatttttagcagagtcagggtttctccatgttggccaggctggtctcaaactccaggtgatccgcccacctcggcctcctgaagtgctgggatccaggcgtgagccgccgcgcccggcctgtgtcgtgttttctgcgtggcctctgtaaacagctgactaggagtctgtgagcatcttcattggttccgctgtgacttgcgtgtgagcatctccagcatcagctttcctcattttcctttaaaatcgcacatgtggatggacaccatggggaccctggaccctgggcagcctcgtcctcacctttctcaccagggtggctgcttgggagagagtgagtttgcgaacttggcatttgttctggctgtaggatccgtttatatatatacatatatttaagtgacagggtctgactctgttgcccaggctggaatgcggtggcgtgatcttagctccctgtagcctcagcctcctgggctcgtggggtcttcccgtctcagcctcttaaagcaccgggaccgcaggcggtcgccactgccatcggatctcaaggctgccacgcagggcgctgcagtaggggaaggggttcctgggactttgaacgctccacctccctcttctccacagtcccccaaccccacctctaactgggagtggatggccacctctttccatcctttgcctggcgcagggtggggagaatgacaggtctccttcccgcatctcggcagctgccctttcgtcggttatatgacgtgggagagaccccggccacccccagggctgtgtgacgtgggagagaccccggccacccccagggctgtgaacaccgccacaggggccttctccactcttcagttttgttagttacgtgacgtgcagggcttttcgttgtaactagtgggggacgtgtggtgaggggggcttctgccgtctcattcaggaccggaacttcagttttcatcgctatttgtcctcctacgcctttggagatggggtctcgctctgttacccaggctggagagcagtggtgccatcagggctcacggcagcctcctcctcctgcagcctccgcctcttgggctcacacaatcctcctgcctcggcctcccaaactcctgggacgactacagacgtgcgccaccgtgcttggcagggttcattcttttcctcacactttatttattggagagcccggtagagcgcccggtttgcacgatctggttgttgagacgttgagaggaaccctgcggagtctgaatctgggtatgggaggggcagtcacacgagttccccggtgtcctcgtctcctccgcacttaggtgcccttaggtggcttgaggaccccagttagactggggccaccctttgcctggctccaggcgggatgtcctttctttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgggactacaggcgcccgccacctcgcccggctagttttttgtattttttagtagagacggggtttcaccgtgtcagccaggatggtctcgatctcctgacctcgtgatccgcccgcctcggcctcccaaagtgctgggattacaggcttgagccaccgcgcccggccgggatgtcctttctatcaggaggctcacagtatctgctttccagtttcttctgatgttaatgttgttgctgctgaacacctgactctgttgattattgggggattgcaagatggtagtgtcccaattctgtcgcttagtcagttagcaggaataatctagcaccatgctctgcatcgcatcagccgtgtggtgatcccattcacacaggaaaggcgggataaatgctggctgtgggccgggtgtgctgtcccgcatctgtaatcccagcactttgggaggccagggcggtcggatcacgaggtcagagattgagaccatcctggccaacatggtgaaaccccgtctctactaaaaatacaaaaaaaaacccaaaacatgagtgggctgtggtggcgcgtgcttgtaatcccagctaccggggagactgaggcaggagaatcacttgaacccggggggcggaggttgcagagagctgagatcgcgccactgcactccagcctcacaacaaagcgagactctgtctcagtaatcaatcagtcaatcaatcagacgtcatcagtgaatgtacgtgagcgtgtagggattggtcctgctccgcgcgccaccctcaatctcacttctgacctccgttctccacctcgaggtgttttcatgtgggcccgtgtgtcctgaccccagcagttgtggcctctttgctgtctgggagtcaggatttttcacactcatgtcctgctccagacctggaatcagccaagtctccaagaagctctggtttcttttcctgcaaggtggtatttcaggacccacgatgcggcaacaggctggtcatggttactgggttggtcattgttactgggtgtttttgtggagatacatccatacgcacagatgtgttcacaaatgttaactctgaaggtaaaccacccggctgggcacaagggctcaccggtaatcccagcactttgggagaccgagacagttggatgacctgaggtcgggagtttaagaccagcctgggcaacatggtgaaaccccatctctactaaaaacgcaaaaattagccaggtgtggtggcacacgccggtagtcccagctagttgggagacagacatgctggaacctgggacacggaggttgcagagagcagagatagtgccgctgcaccccagcctgggtgatagggtgacaccctgtctccaaaataaatagataaataaagggaaaacgcctcctcttggtgtctccagtttggactggctggcatgggttccggtagcctcttcccccatggtggcgtccttttttccatgccgggaatcctggttctcaagggcggggttgttggtctgagtgtgacacagactgtgcctttgctgcctttctcttgcccagggctgaacatggagctggaagacattgcaaagctgaagagtaagtgctgccctccccaccttcttgcggctgggtggggcctccccctgatgcctcctcaacccgacctgcagtgatcctgctccgcctggagggggccatcgatgccattgagctgcctggagacgacagcggtgtcaccaagccagggaggtgagaggcggggagccagctgcttcactggaggcccagcctggagctagagataggccatggtgcagtcctaggctgtcccaccagtgaggcctgttttcaggcccgttttccctttttgagacctggtaggagcacctggtttgcatgatctggttgttgagacgttgagaggaagagcacacgcatccacggaacagcacacgcatccacggaacagcacacgcatccacggaacagcacacactcccacggaacagcacacgcatccacggaacagcacccactcccacggaacagcacacacacccacggaacagcacatgcttccacggaacagcacatgcttccacggaacagcacacccccacggaacggcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacaacagcacacactcccacggaacagcacacgcccccacggaacagcacacacacccacggaacagcacacactcccacggaacagcacacgcccccacggaacagcacacacacccacggaacagcacacacgcccccacagaacagcacacacccccacggaacagcacacgcacccatggaacagcacccacggaacagcacacactcccacggaacagcacacgcacccacggaacagcacacacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacactcccatggaacagcacacacacacccacggaacagcacacacgcccacggaacagcacacactcccacggaacagcacacacatccacggaacagcacccacggaacagcacccacgcccgcatggaacagcacccacggaacaacacacacccccacggaacagcacacacccccacggaacaacacacactcccacggaacaacacacacccccacggaacaacacacacccccacggaacagcacacacacccacggaacaacacacactcccacggaacaacacacacccccacggaacaacacacacccccacggaacagcacacacacccccacggaacaacacacacccccacggaacagcacacactcccatggaacagcacacacacccacggaacagcacacacacacccacggaacagcacacacacgcccacggaacagcacacacccccacggaacagcacacacccccacggaacagcacacacccccacggaacagcacacacacgcccacggaacagcacacacgcccatgcggggccgctgggtttcctgcagtttgtcctcctccaggcctttccctggacctcctcaggtgcctgttagaatgagaccttcttgttaggacgatgagtgtcccagccaccacctcttttggactccgggaggcccggaacattctgaaggctccgtgggtctcctgtcttctccgcggccagggcagcagcatttgttgtctgtcctgcaggcagatgagggtcaggcctggggcccgtgtggggctccactgagtgcacagccgccaaggcgagcgcgttctgcctgagcgctgaccttgtggcgactcccagttgcttccaggtggagccagtgtcctcagcgtaatgctcaaggctctggcgtggctcctcggccacctgcacccccaggggcccctcctgtagcttctgctgccgccatcactgtcactctcccaaggctttggggactctgcccagagccgctgcctcccagaagcccctgacgaccccccggcgaccccatccctccctctgatgacggcctctgccccgaggcggcttttccttggtgctgttcttgtgccggccaggcctccacttcccatctggaggctcctgagggcggaggcctctcacccccaatgccggcgtcccctggagggctcagtttgtttccgagggaaggaaacttccacagttgctgccttcagttcccaaagctgcagcctgatcccccgtccaggctcgagcctgccttcttctcctcgcagctacatctttgagctgtttgctgaagcccggatcacgtttcagaccaagggctgcatcctggactcgctggaccagatcatccagcacctggccggacgtgagtgccggggcggggtccttggtgtgggcagacgtggcgtagggggtgcagcaggcctccatcctggcagtcagggctcgcctggccatcacctggccgtcagcaggaacaggctcacagaacctcacctggtcggggcgtggaggcgttagtgccggttgcccactgccgtagagcctgtcccagttctgcagctggcggcttcgtcctgcagactcatcccattattccccttttaaggaagagcagcccaaggccctggctgggttgtggggcctcaggcttctccacacaccctgagttctgcttctcagagctgtggggtccagaggctttgcccagaggcagtgtcgtcatgggctgctctggtttgagatgccggcccgccggggcctctgctgtgctcccaggtgctggggagggcggctcttgctgcttcaaaccttaggtgaccatagagcctcttttcaagttccaccggcccccttggagacgctgtccctgcctggctgctctcctggctgctgggaagggcaggcgagctgcccgccctgagtggacactgcgctccaccctgggccccccgttgggcaggagatggagcttggcagtcgggctgagctggcttatggctggaagggcgggggctggggtcggggcctcccctgcctgcagtgcgggtgtcagcgcccggcatgctacctccccaggtgctggagtgttcaccaacacggccggactgcagaagctggcggacatcatccaggtggggcctgctcctctggcgtctccttccctgatggaagccgggcgggtgccttctcctgctgtattagttactgactctagacgtgggggtgggagaacggcccccacaccacctgtttccgactggcaaacgcttggctcctttcctgtgacccaaacccacactgggcctcccgcggggatgggggaggacgaggtctggtggcccgtgcccagggtgacgctggtgggggaggacgtgaaggggagtgggggccagggagccgctcctgccctgtccgggccctcaggccggggggacccactcctggcagccccagctacacgcagatgaagagctctggacacacgtagcttcctgaacagcttctccagggacagacaaacggggaccctgcaggttgccggcgggggtgtccctgcgagcccatgatgggggatgcggccccggcccccttcttggccccgtcctgccctgtgatgcccgtcccctcccatgtgaggttgctcctggctctgtgcctgtggcagcacctgctttccctgaggcagacgggcttgtgcggtttgtggagggcaagccccatttttgggtgctctgggcacacacttgcctctcctggacctcggtgcattttttagaagcagacccacaagtccctgactgggaggccatcagtccttggggaaaccgaggcacaggtggctgtgtggagccatgtgaggctgcctctgtgctgcccacactgaacctcctggccacacgaggtcatggcagggttaggtggggctgcccagctctgtgacaacttcgtgagtgtccgcctggggtggggggtgcttgagctgggggaggccgcagaagaaccctggaatgtaccgccggcctgtgcatgcagtgcggtgctctgtgcatgctactgggcgtggagctgcaggccccaggacgaccaggcactgccttcttcatagacgtggctctgagcaggggcgagacctgggcagggaccaggtggggtgggcacagcaggtggtagggcccaggctcagccctacctccaccgtggccttctctgcggccagtgatgccacagccagtgtcttctctgtgtggcagctggggctggaaagacggcactgctatgtctcccagctcctccccaaaagcacggtgggcatcccagggccagacccctctctctgtggctcctgcctgccaagggctgctttgccgtcccgcctggagcgtggttggctcttcaagcaggaggccgtgcacctgccaggcggacctgcttccatgtcctgacgggtcactcaaagcacctccagcacatggccatcgaggcagccctgcagcccagggcctggagggcaggtgtgagctggcccgggcctgcccctccctggaatgtggcttcccaggctccagtatggagcagtgtccacactctggaaccaaatcttgttcaccccattggtttggaatttcctttggctgctgctctaagtagccactgggagatgactcagctcctcccaaccctggggactgcacaccgtgttcaccctcggctgctcctgagtccctgcaggtgtgggcactgctcatagggctgctgctgggctggacttgcagccctgaggcacaggtgaccctggggacagagtggggctagagtggggctagtggccccaggaagaggatgtgggatgcacggttcagttggaggcaagtccgggcagggccaggctgtgtgctggggaggtagagcttagctggaggcaaactctgggcagtgctggcccgtgctgggcagccagtcaccgctgggcccctgtgagactagtgggtcccctttcttctctgcctgagctgccctggggctgaagcacggggcggtttgtagtggtgtttttttgagacagggtctcgctctgtcaccccagcgggagtgcagggccttgtcaccagctcactgcagcctcagcctcctgggcccaggccatcctcttgcctcagccacctgagtagctgggaacacaggcgtgcaccaccacgcccggctaattttaaaagcttttttgtggagatggggtcttggcgtgcaccaccacgctcggctaattttttaaatttttatgtggagatgggtcttgccatgttgcccaggctggtctcaaagtcctgggctccggcagtctgcccaccttggcctcccacagtgctgggattacaggtaagagcttccaggcctgcccatcagagggctggctttttgagggaagctgtttcctgaggcagcctcagcccctgctcatggcagcccctggtgtcatggtgaatttgttctgttgtttccatccctgtgggccaaatgctttggtaaaacgcaaggccttgtatttagctgtcttgacagtgaaaatggctgggaaggaaggaaggaaggaatggaaggaagtttctctctcccgtgcatacccaggcacgtgcacacgtgcgcagtccacgcacgctgccggtctctgtcccagccgcggtgccggcccccgtgttctccagttcagcttctccgcatttctgtgaacgattccggcttcttgatgttcccagcagaactccctcaagccttcggcttctccagggctctgacggcggctgggctgacgtggccgctctgcggagcctgttggggcttcacgttcctgttctggccatttccggctctttttccgggaagggtcatcacgcgctcttttcctagtgcaggcatcgtccctttcccgttgatttgcagggttctctgtaagatctgaggaacacgtgccaacatattctttcctggtctcgtgctggtcattttttccattttcacgacaggtttggtgaacagatgtttccttgtcatcagacttttgtcttgagcttgtatcccccgaccaagatgctaaactgggtcttgtgtgttctccaaactgcactgtagggtggcggagctttgtgtcggggcctccgcaccttccggcgtcacctgtgggggtgtgaaaggcagactctacctggatttttcccagcacgccacaccggtggttccgtctgctgaccgagtggctcggcttctcccaactccgctgggcacctgccacactgttttcctcacggttttgttcgtggtggttttgtcgtaagtcctggtgtcggccagaaccaatttctgtttttgtttgtttttgagatggagtttcgctcttgtcgcccagggtggagtgcagtggtacgatcttggctcactgtaacctccgcctcctgggttcaaatgattctcctgcctcagcctcctgagtagctgggattacaggtgcttgccaccacgcccagctaattttcatatttttagtagagacggggttttgccgtgtgggccaggctggtctcgaacccctgacctcaggtgatcctctcgcctcagcctcccagagtgctgggattacaggtgtgagccgccgcgcccggcctcttgttcttcttaaacctgccctgatgttttctccatgatgcatgttggagtcaacctgtctgcttcctttaaaaatcttgctgtgattttgactggaatgtgtggaattcctgtgtttttgagtcagtgtctctctgttgcccagtctggagtgcagtgggaccatcacagctcactgcagcctcaacctcctgggctcaggggatcctctcagcccagcctcccaagtagctgggaccacggcacgtgccaccacgcccggctagttttcatatttttttggtggagaccccagggttgcaccatgttgcccaggctggtctcgaactcctgggttcgggcagttcgcccctctcggcctctcagagtgctgggattacaggcgtgagccactgcactaggccatgttgaatttctagattaatttgggacccttaggggcacagagaggagggctgggccagctggtgggaggagaggcctctcgggctgccgcatttccagtgcacggagatggcctgcgtagggggaacagagctcactgggggtccctccagggaggagaaagggtcaggcaggtgccagctcctgtccatcagcctggggctgcatgatggcaggggctggtgaaccgatgacctctgggtgtcctgtgaccttccgtgtgtggggctgatgccgcagaagatcaggtggcctccagggctccgggctctggcctcaggctcctcacgggactgcacctcctctctttcagattgtgtttagtgtggacccctccgagggcagccgtggttccccggcagggctgggacccttacagtcctataaggtaggggccacctccaggaggcaggtggggctgggggtcctgcagtcctgaaaggtaagcggctgcccccaggacacgggcggggtctccacacttctggtcctgtcccctccaggtgcacgtccatcctgatgctggtcaccggaggacggctcagcggtctgatgcctggagcaccactgcagccagaaagcgaggtacagacctgggcccacactctccccgcccgcccgggtgtggtgctcagcaccagcatgccacaggctaggcacgtggccagccgtgggtctcctgcccccctgggcctggccaccttctccatgtccaggccaatccagagcgttctcctcactgtccctctgaaggctggagttactgagagacatgggagagggcctgatggcaccgtgacctgcccagagtcacctggttggtggcggcagagccacagctcagccaggccttcctgctgggacacactggtttatgccaaggccgtcagcacggagccccacagtgaggcacagctttccctgctgcctccacccagcgcctggctgggccaagcctcagggtctcatctgaagggggcccgactggccctgttgtccgaagcctctggtgcgctcggccctgaggccccacgtgccttgttggcttcctgtgctctgtggcgtcttcgagtcggtgctgccgggtacctgtgtggatggggcccgtgagtgggccctcggctccgtgtccagagtcctgtggttcttggagtgtgtccggccccacccggccatgtggtgtccaaggggggctttgtggcggcagcggtgggagctgctgccccttctcgtgctccagtggggccttgcctctgggcttggttcgttcctctctggaacgttctttctcagagctctctggcccgcggtggcacgacgtggccctggctgaagcagcccccgcgtggttgctgtggttggtctgcctggccgagccggaaggggaggcccgggagggcgtcagggttgcgtggcttgacccccggtcggtggctcggtgatggcctcgtggcaaggcgtctcccagcaggaagcgtccatcctggggggtcgtgggaggcggcgcgtgcctgaggcagctggcgcacagatcctgagggcctggaatctatttgttccattgacctgtgacgtcacttgacttctgagaagcagccgctccctgcgttgggtgtttgtaggaaatgagttcctggaggcggaggtcgggaagttcccccgctgcagggcacgctcagccccaggaagcggggtgacctttgctgggagaggtgccttgtccctgctgaccccgagtcacagtcagcgtcgttctgcttgtgccgtgtttaattcctggtgtccggtgcccaggctcagtctcctccccaggccgcggccgctgcggtcagtcagtcaagcacttctaggaacttcctttgaaccggtgcccttgttcgctgctgacgccactcggaggcttcctggcggagcggggaactcgcacttcctcttgtgcgcgtccctggaatgtcgggacgctggcgcttgcttcccgtgcgctcttggcgggatgagcttgccctgagctgcatgtcactgtttctgcagaagtttagggttggagttggctgacctctctggaggtgtccccagcatctgcctggggtctgcctcgtactcccgggacccccagtcccccagaggggaccccagctggcagactgcgctcacactcagggcagcctcctttgttctgacttctgcacgctgggcctgggtggctgtgggcggctcacttgcttgatgccagtgggtgagagggtgatgggcagagaggcaggcggtcaggcccccagtcccgtcctcacaccctgtgtcccctgctcccctccgccccacagggaaggtgttgagctactggtgcttcagtcccggccacagcatgcgcgagctggtccgccagggcgtccgctccctcatccttaccagcggcacgctggccccggtgtcctcctttgccctggagatgcagatgtacgggccacccctgccaggggcctaagcactggtgacgcctgtgacgttagtggggtgggagtgctgggggtccccatgagccggggtgctgggggtcttgggcctcactgggggtctcgggccttgagggctaaaggggtgctggtgtccttccccactgtctgtccctccggccacgctcagccctttcccggtctgcctggagaacccacacatcatcgacaagcaccagatctgggtgggggtcgtccccagaggtcccgacggagcccagctgagctccgcgtttgacagacggtgagggcctgtccctgggccgtgctggggtgcgaggtcggggagtactgagccctgaggccctgagcagtggcctctcggctctgtgcaggttttctgaggagtgcttgtcctccctggggaaggctctgggtaagtgcccggcacaccccagctgtgcccatcctggatcctggaccctgctcccaagagctggtagggaccccggaagacgccccgcccctgccctgaccccagcccctgcacttccaggcaacatcgcccgcgtggtgccctacgggctgctgatcttcttcccttcctatcctgtcatggagaagagcctggagttctggcgagtatgtctcccctgtgtgtcctgggtggggtgggtgagggcagggctggagcacgaagcaggcagtggccacagctcctgcctgccctcatcggatcggcggcgtaaccagggctgccgtgtccctgcctcttcctcccacaggcccgcgacttggccaggaagatggaggcactgaagccgctgtttgtggagcccaggagcaaaggcagcttctccgaggtcagcacttggccggggctctgggtctgctgccccctcgtgcctcccgtgcccctcacagcttccccgaggctgaccaccagccctgaccacgggctctggcggctcccgcctgcctcttcagggcccctgcgtttccttcctggccctgagtgttgcctcttatcttacaaagcccccagcactgggtgggtgtggcgacagtggtcctcctgcctgaggagctatagccggcggccaccctggccctgggattccccgtgttttctgggaagcactgagcaggcgcaggatcagcctgggatccgtgccaggaagaagtttccagaacccagttggccttcctggctaggatagtccttcatcttggaggatgagacctgggtctccctcatgggggaagaaggggccgaggtgggctccaggctgagcctgaccaactttccttccagaccatcagtgcttactatgcaagggtcgctgcccctgggtccaccggcgccaccttcctggcggtgtgccggggcaaggtgagctctctggggccctctgtcctgacccggttgcctgttccccagtgggtgctcatggctcctcagctgactttgggccctgggggctgcctggtcccctccttgggccccacaagagcaaagcactggtggcctgctgggagcgtgccccgctctgggtcgggcaggcaggatgcaggaggttcctggccacgagagttggaggtggcgtctgggagctgtggaccccaagtggggtcctgacccatagatggggcttcctcccacccctggttggagcccccatggccccaggcagctccctggtgtgtcccccaggccagcgaggggttggacttctcagacacgaatggccgtggtgtgatcgtcacgggcctcccgtaccccccccgcatggacccccgggttgtcctcaagatgcagttcctggacgagatgaagggccagggtggggctgggggccaggtgagttacagcagggcggggctggggtaaggcaatctggcgactgagcccccctgtgaccacgggagcccctgtgaccgagcctcctcgccctccagttcctctctgggcaggagtggtaccggcagcaggcgtccagggcggtgaaccaggccatcgggcgagtgatccggcaccgccaggactacggggctgtcttcctctgtgaccacaggtgcgtgcagtccgggggcaggcgtggcgccaggggacacggccacatcccactgggccctggactccttccccacatgaggccccgtctcccccagagcctctctggcgactcggggtcagcctgaggcccctgcggcagatgagggtcttcacttgggtgaactgaccccctgaagcggctgtgggcagggcagcagggctgtggccaccccccaggttcgcctttgctgatgccagagcccagctgccctcctgggtgcgtcctcacgtcagggtgtacgacaacttcggccacgtcatccgagacgtggcccagttcttccgtgttgccgagcgaactgtgagtttctgcccagggaggggatgagggtgttgtccccagaggagccagaaatgggcccacccacccccagtggttctgcagatgccagcaccggccccccgggctgcagcacccagtttgcgtgaaggagaagatgctgtcagggaggccaagtcgcctgaccccctcctctccaccaggaaagctaagagtctggacctgcatgtccccagcctgaagcagaggtcctcaggtgcagatggccagtgcgggacgggcagtgtggggcgggcggggcaggcggattggggcgggccgtgtggggcaagcagcggggcaggtggtgtggggcgggcggcaccaggcgcccagggtggaggcggctcacctggctttatgcactgcccctcccacctccaaaggctgtgtctgcctcctagggtggggttcccacgggctggaaccctcccctacaggcagagaaggcctcaggcaaagacgcgccccaaggctgggactccccctaatagcagggaggacacccacaggcaggggcccccaaatgctgggaccctccctcaggaggggctccgtcataggcagggacccccagctggggccccgcggtgggcttcactgcgcactcgggtactctgcaggatcaccagctgccggggaccccgagagtagcctgtgtgtggagtatgagcaggagccagttcctgcccggcagaggcccagggggctgctggccgccctggagcacagtgaacagcgggccggggaccttggcgaggagcaggtgcagttccagggccttgggatggacacagaccctctgtctcctgaggccaaccccaccccgcccatctgacttcaggcacctccccacacacccctgtaaatcccctgcctgtggggcgggcaggcaggtgagggatcccagctgcctggctgtgtgtgtgccctccacctcgcctcacccacaggctgctggctcccaggtggtgcatgccctggccctcagcgggtgcccccacatcactttgattctctggcaggtcagcttggctcagggcactcaaggtcgggtgcccctgcccctggctgtgcctgaggctggcctttctccaggaatgtgctgcggttgggacccaggttccttcctccttggggccttttgccccagaagcccataatttctcagctttctccctgcttcctgctgggggccgttctcttgctgcctgtccctgcccttcaggctcctggagtgagccccgggtgcaggcaccagacacctgtgtccccttcctgccagcccctcgctgtggtcggactgtccttcctggacctgctcttacgtgtcaccacctgtgagcctcatgagccgctggcgtgacttggacaggaccaagttgtgggactgtcaccagggtgtgccgcgcccctcccccgaccttcgtcttggctactgctcagggctccttggggccatcttccctgtgggcccaggtgctttggggccctggagtgtgtggttggaaccacagctgtgtcctcctcaggcccacagctgctccaccctgtccctcctggctgagaagaggccggcagaggagctgcgaggagggaggaagaagatccggctggtcagccacctggtgcgtgagctgtccctgcacctgtgccgaccaccatagacacgtgtgggaacccggccctgggtgcccccagccaagttgggatgtgggcccgatgggaccagggaagttacccccgggagctgatgtccagggcggctgtgaagctgtcggcccaagggctcaagggcagctgggatcaggccccgcccttgggcaggcttggtttgggggctgcagcactgggtttggccctggcatttccctcaagtgtggacgtgcacctgcctcattcgaggggcgcagcccattcccagccttggatgaaagaaccgagttatagtcggagccaggaagccccctgcctgctggaaagccccaagtgtggcagcctttgtccatgtcccttggcttctgggaagaactgggtggtgcccaggcagggctggtgccatcaggaagtgggcggctgccgaggggcctgggctgttgagggcctgggtggggagtgccggggctgcccctgccttggtttccacgtttccgtgttggtctggggtgtgtggagagatgagcaccgcttcttgggaagcccctccctatgatctgccatcctggagcctcagctgtgtccgctgtggggtgggggctttagggaggaggagagaggacccccagcttgagggagagccagtctaccctttgcccattcgctgccctcggttccctgccagcctctcactgtgtgacccagacaggcccagccccgcagctttcctcccgcagcacctccgtgtccatccagccaaccagtttctcaggcagcagcccccgctcagcaatcactgtcccggggaacacccaatgctccaaggaaggctctgcagctccagggacgggatgatgaggctggccctgatggagcctcgggcctgtgtgctgcaggaggagcccgtggctggcgcacagatggacagggccaagctcttcatggtggctgtgaagcaggaactgagccaagccaactttgccaccttcacccaggccctgcaggactacaagggttccgatgacttcgccgccctggccgcctgtctcggccccctctttgctgaggaccccaagaagcacagcctgctccaaggtgccctggcttgcaggggccgcccatcctgagggcagtgctgccgctgtgtggggtgggggccatctgggtccaaggttgtctctgttctctagagaaaagggggcggatggggacagacaccccttcctctacaggcttctaccagtttgtgcggcctcaccacaaggagcagtttgaggaggtctgtatccagctgacgggacggggctgcggctaccggccagagcacagcgttccccgaaggcagcgggcacagccagccctggaccccactggtaaatggggcctcgggtgggaccctcagactcctccgtggaaggcaccggagtcctgggctggttggggtgggcatccctggggccctgcgtggccccgcctctctgttcccgtgtgggagtgatgggggccaccccccaccaccagcagcagtatcacttgttggggagaccctgtgcaactccacgcagggccctgcccttcccatagcccctgacccctacgcacagccctgtccagctgccgcacgtgtcccctgccccccatgcacagccctggggagtccactgcacacgccagggtcctagacccctagacccctagatccctgtcctcccgtcctccctacttctgcctttgcctagggtggagtccaagcctccagaggtgggagtgtctgtgttcgtgcttgttaacgaacagcccccatagagctcctccagtgtagggttcacccagcggggaacctaacctgttgggaagaccccagaccccttctgggcttggtacccaccctggggctctaagctcctgagcagggctttggcctgcctgccactgttccagcccccatccagcagttggtgtctcctctgctgccccggcacctgggcacgtgtgcctgcctgggttttcccgccctggtccgaggtgggtgaggcctggcctccccagccagcccttccccctccccgggggaactttccaggtgcttgggaccagctttgtggctctccatctcttcatcaggaaggacagcgctagatcccaagctgactgtgtccaaggctgcagcccagcagctggacccccgagagcacctgaaccagggcaggccccacctgtcgcccagaccaccccccacaggtagctgagtcctgaactgtgtgcagcctgtgacttggtgggttcctcagtggcttgatgaggctaacacttgagtgtggcttgagcctttgaaccggaggtgccagtggagctgcccctgtggggagcatctcctggtgggggctcccggttctgtaccccgcagttgtcctgagcagctctccaggagttcgtgggaggaagggcaggcagggtggtgggactctcagtcctctgccccagcgccactctgagccatgctactcccacaccaggaggccctggcagccacccacagcaggggtccagagcgcccagagcagggaagcagggccagcgcgccgtgagcgcctacctggcagatgtccgcagggccctggggtctgcaggctgtagccagctcttggcagcactgacagcctacaagcaagacgatgacctcgacaaggtgctggccgtgctggccgccctgaccactgcaaagccggaggacttccccctgctgcagagcaagtggcctgggcgtgggggacagccagtggggtgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggcaggggcagggtggggaccgtgccgggtctgagtgaagctccccgcagggttcagcatgtttgtgcgtccacaccacaagcagcgcttctcacagacgtgcacagacctgaccggcagaccctccctgggcacggagccaccaggaccccaggaggagagtcttgccgtgcctcctgtgctcacccacagggctccccaaccaggtagggcgcctgcctggctgctcctgacagtgccccaaccgcacgcagccctgggagtgagcagcaaagccccaggccctgctcagactcagtctccgtctccaggcccctcacggtccgagaagcccggaaagacccagagcgagatctcgaccttccttagacagaggccagcagggactgtgggggcgggcggtgaggctgcagggcccagccagtccccaggacctccccacgggcctgcagcatctgagtggggtgagcctcctgggagagaccttgctgggcagcaggctgcgggagctccgagcgggcccctctcagcaggctgtgtgtgccagggctgtggggcggaggatgtggtgcccttccagtgccctgcctgtgatttccagcgctgccaagcctgttggcaacggcaccttcaggttggtgcctggtcactacagttcctgctgggtgtagccccaggtggtgggctgagggggaaagggccgggtatagccccaggtggtgggctggtggcaacgcctggcaggtgcgtccagcgggccggttgtcttacaggcctctaggacgtgcccatcctgccacaccgcctccaggaagcagagcgtcacgcaggtcttctggccagagccccagtga

SEQ ID NO: 3 - DNA, 18 bp, Homo sapiens
  source 1..18: mol_type = other DNA
gaccactgtccttccatg

SEQ ID NO: 4 - DNA, 46 bp, Homo sapiens
  source 1..46: mol_type = other DNA
ttcagagattcaagttataataaagctcttcttatattgaggggga

SEQ ID NO: 5 - DNA, 18 bp, Homo sapiens
  source 1..18: mol_type = other DNA
aggaatagggttggtttt

SEQ ID NO: 6 - DNA, 17 bp, Homo sapiens
  source 1..17: mol_type = other DNA
ccttactacctgtcccg

SEQ ID NO: 7 - DNA, 20 bp, Homo sapiens
  source 1..20: mol_type = other DNA
acaattacttgttggatgcc

SEQ ID NO: 8 - DNA, 18 bp, Homo sapiens
  source 1..18: mol_type = other DNA
agcttctaacccaaccag

SEQ ID NO: 9 - DNA, 21 bp, Homo sapiens
  source 1..21: mol_type = other DNA
tataaacctaaatgtaaaagc

SEQ ID NO: 10 - DNA, 20 bp, Homo sapiens
  source 1..20: mol_type = other DNA
ttcaccaaaatttaaagctt

SEQ ID NO: 11 - DNA, 26 bp, Homo sapiens
  source 1..26: mol_type = other DNA
ctttgaccagagtatgtaaaattctc

SEQ ID NO: 12 - DNA, 18 bp, Homo sapiens
  source 1..18: mol_type = other DNA
aagacgtgttcaaagatt

SEQ ID NO: 13 - DNA, 17 bp, Homo sapiens
  source 1..17: mol_type = other DNA
ggacctactgttttttg

SEQ ID NO: 14 - DNA, 19 bp, Homo sapiens
  source 1..19: mol_type = other DNA
ggacctactgttttattcc

SEQ ID NO: 15 - DNA, 22 bp, Homo sapiens
  source 1..22: mol_type = other DNA
gtcccttctcttcctcctgtag

SEQ ID NO: 16 - DNA, 19 bp, Homo sapiens
  source 1..19: mol_type = other DNA
cgtgatctttgacgaagct

SEQ ID NO: 17 - DNA, 16 bp, Homo sapiens
  source 1..16: mol_type = other DNA
cgcaaacctttctgga

SEQ ID NO: 18 - DNA, 23 bp, Homo sapiens
  source 1..23: mol_type = other DNA
agcctgtgtgtggagtatgagca

SEQ ID NO: 19 - DNA, 20 bp, Homo sapiens
  source 1..20: mol_type = other DNA
cgtttccgtgttggtctggg

SEQ ID NO: 20 - DNA, 19 bp, Homo sapiens
  source 1..19: mol_type = other DNA
gactacaagggttccgatg

SEQ ID NO: 21 - DNA, 20 bp, Homo sapiens
  source 1..20: mol_type = other DNA
agtttgaggaggtctgtatc

SEQ ID NO: 22 - DNA, 17 bp, synthetic construct
  misc_feature 1..17: note = Synthetic oligonucleotide
  source 1..17: mol_type = other DNA
catggaaggacagtggt

SEQ ID NO: 23 - DNA, 20 bp, synthetic construct
  misc_feature 1..20: note = Synthetic oligonucleotide
  source 1..20: mol_type = other DNA
agctttattataacttgaat

SEQ ID NO: 24 - DNA, 16 bp, synthetic construct
  misc_feature 1..16: note = Synthetic oligonucleotide
  source 1..16: mol_type = other DNA
cgggacaggtagtaag

SEQ ID NO: 25 - DNA, 15 bp, synthetic construct
  misc_feature 1..15: note = Synthetic oligonucleotide
  source 1..15: mol_type = other DNA
cgggacaggtagtaa

SEQ ID NO: 26 - DNA, 19 bp, synthetic construct
  misc_feature 1..19: note = Synthetic oligonucleotide
  source 1..19: mol_type = other DNA
gcatccaacaagtaattgt

SEQ ID NO: 27 - DNA, 18 bp, synthetic construct
  misc_feature 1..18: note = Synthetic oligonucleotide
  source 1..18: mol_type = other DNA
gcatccaacaagtaattg

SEQ ID NO: 28 - DNA, 18 bp, synthetic construct
  misc_feature 1..18: note = Synthetic oligonucleotide
  source 1..18: mol_type = other DNA
ggcatccaacaagtaatt

SEQ ID NO: 29 - DNA, 16 bp, synthetic construct
  misc_feature 1..16: note = Synthetic oligonucleotide
  source 1..16: mol_type = other DNA
ggttgggttagaagct

SEQ ID NO: 30 - DNA, 20 bp, synthetic construct
  misc_feature 1..20: note = Synthetic oligonucleotide
  source 1..20: mol_type = other DNA
gcttttacatttaggtttat

SEQ ID NO: 31 - DNA, 19 bp, synthetic construct
  misc_feature 1..19: note = Synthetic oligonucleotide
  source 1..19: mol_type = other DNA
catgttcctttctataact

SEQ ID NO: 32 - DNA, 19 bp, synthetic construct
  misc_feature 1..19: note = Synthetic oligonucleotide
  source 1..19: mol_type = other DNA
agctttaaattttggtgaa

SEQ ID NO: 33 - DNA, 20 bp, synthetic construct
  misc_feature 1..20: note = Synthetic oligonucleotide
  source 1..20: mol_type = other DNA
ttttacatactctggtcaaa

SEQ ID NO: 34 - DNA, 18 bp, synthetic construct
  misc_feature 1..18: note = Synthetic oligonucleotide
  source 1..18: mol_type = other DNA
ttttacatactctggtca

SEQ ID NO: 35 - DNA, 20 bp, synthetic construct
  misc_feature 1..20: note = Synthetic oligonucleotide
  source 1..20: mol_type = other DNA
gaattttacatactctggtc

SEQ ID NO: 36 - DNA, 19 bp, synthetic construct
  misc_feature 1..19: note = Synthetic oligonucleotide
  source 1..19: mol_type = other DNA
gaattttacatactctggt

SEQ ID NO: 37 - DNA, 20 bp, synthetic construct
  misc_feature 1..20: note = Synthetic oligonucleotide
  source 1..20: mol_type = other DNA
gagaattttacatactctgg

SEQ ID NO: 38 - DNA, 17 bp, synthetic construct
  misc_feature 1..17: note = Synthetic oligonucleotide
  source 1..17: mol_type = other DNA
atctttgaacacgtctt

SEQ ID NO: 39 - DNA, 18 bp, synthetic construct
  misc_feature 1..18: note = Synthetic oligonucleotide
  source 1..18: mol_type = other DNA
acaaaaaacagtaggtcc

SEQ ID NO: 40 - DNA, 18 bp, synthetic construct
  misc_feature 1..18: note = Synthetic oligonucleotide
  source 1..18: mol_type = other DNA
ggaataaaacagtaggtc

SEQ ID NO: 41 - DNA, 18 bp, synthetic construct
  misc_feature 1..18: note = Synthetic oligonucleotide
  source 1..18: mol_type = other DNA
agcttcgtcaaagatcac

SEQ ID NO: 42 - DNA, 16 bp, synthetic construct
  misc_feature 1..16: note = Synthetic oligonucleotide
  source 1..16: mol_type = other DNA
ggtgggtggatgtttc

SEQ ID NO: 43 - DNA, 16 bp, synthetic construct
  misc_feature 1..16: note = Synthetic oligonucleotide
  source 1..16: mol_type = other DNA
ggtggtgtggagaagc

SEQ ID NO: 44 - DNA, 17 bp, synthetic construct
  misc_feature 1..17: note = Synthetic oligonucleotide
  source 1..17: mol_type = other DNA
gctcatactccacacac

SEQ ID NO: 45 - DNA, 20 bp, synthetic construct
  misc_feature 1..20: note = Synthetic oligonucleotide
  source 1..20: mol_type = other DNA
catcggaacccttgtagtcc

SEQ ID NO: 46 - DNA, 19 bp, synthetic construct
  misc_feature 1..19: note = Synthetic oligonucleotide
  source 1..19: mol_type = other DNA
gatacagacctcctcaaac

SEQ ID NO: 47 - DNA, 17 bp, synthetic construct
  misc_feature 1..17: note = Synthetic oligonucleotide
  source 1..17: mol_type = other DNA
ggtggaggtggtgctgc

SEQ ID NO: 48 - DNA, 16 bp, synthetic construct
  misc_feature 1..16: note = Synthetic oligonucleotide
  source 1..16: mol_type = other DNA
aggtggaggtggtgct

SEQ ID NO: 49 - DNA, 16 bp, synthetic construct
  misc_feature 1..16: note = Synthetic oligonucleotide
  source 1..16: mol_type = other DNA
tggtgtgggagtagca

SEQ ID NO: 50 - DNA, 16 bp, synthetic construct
  misc_feature 1..16: note = Synthetic oligonucleotide
  source 1..16: mol_type = other DNA
cgatggcgagaaatta

SEQ ID NO: 51 - DNA, 20 bp, synthetic construct
  misc_feature 1..20: note = Synthetic oligonucleotide
  source 1..20: mol_type = other DNA
taattcagcaaaaaagccca

SEQ ID NO: 52 - DNA, 17 bp, synthetic construct
  misc_feature 1..17: note = Synthetic oligonucleotide
  source 1..17: mol_type = other DNA
aagaatctgacacccca

SEQ ID NO: 53 - DNA, 21 bp, synthetic construct
  misc_feature 1..21: note = Synthetic oligonucleotide
  source 1..21: mol_type = other DNA
agacagccaagaatctgacac

SEQ ID NO: 54 - DNA, 17 bp, synthetic construct
  misc_feature 1..17: note = Synthetic oligonucleotide
  source 1..17: mol_type = other DNA
cagccaagaatctgaca

SEQ ID NO: 55 - DNA, 15 bp, synthetic construct
  misc_feature 1..15: note = Synthetic oligonucleotide
  source 1..15: mol_type = other DNA
acaggaacccgacag

SEQ ID NO: 56 - DNA, 21 bp, synthetic construct
  misc_feature 1..21: note = Synthetic oligonucleotide
  source 1..21: mol_type = other DNA
gttactctcttgtttcttcac

SEQ ID NO: 57 - DNA, 19 bp, synthetic construct
  misc_feature 1..19: note = Synthetic oligonucleotide
  source 1..19: mol_type = other DNA
ttactctcttgtttcttca

SEQ ID NO: 58 - DNA, 19 bp, synthetic construct
  misc_feature 1..19: note = Synthetic oligonucleotide
  source 1..19: mol_type = other DNA
gttactctcttgtttcttc

SEQ ID NO: 59 - DNA, 16 bp, synthetic construct
  misc_feature 1..16: note = Synthetic oligonucleotide
  source 1..16: mol_type = other DNA
cgtgggtggagaagca

SEQ ID NO: 60 - DNA, 16 bp, synthetic construct
  misc_feature 1..16: note = Synthetic oligonucleotide
  source 1..16: mol_type = other DNA
acgtgggtggagaagc

SEQ ID NO: 61 - DNA, 17 bp, synthetic construct
  misc_feature 1..17: note = Synthetic oligonucleotide
  source 1..17: mol_type = other DNA
cagaaactgtaagggca

SEQ ID NO: 62 - DNA, 17 bp, synthetic construct
  misc_feature 1..17: note = Synthetic oligonucleotide
  source 1..17: mol_type = other DNA
agggatagcagggaagg

SEQ ID NO: 63 - DNA, 17 bp, synthetic construct
  misc_feature 1..17: note = Synthetic oligonucleotide
  source 1..17: mol_type = other DNA
gcttaaacacagacaga

SEQ ID NO: 64 - DNA, 17 bp, synthetic construct
  misc_feature 1..17: note = Synthetic oligonucleotide
  source 1..17: mol_type = other DNA
tgcttaaacacagacag

SEQ ID NO: 65 - DNA, 18 bp, synthetic construct
  misc_feature 1..18: note = Synthetic oligonucleotide
  source 1..18: mol_type = other DNA
cagggcagggaagaacag

SEQ ID NO: 66 - DNA, 16 bp, synthetic construct
  misc_feature 1..16: note = Synthetic oligonucleotide
  source 1..16: mol_type = other DNA
catggaaggacagtgg

SEQ ID NO: 67 - DNA, 17 bp, synthetic construct
  misc_feature 1..17: note = Synthetic oligonucleotide
  source 1..17: mol_type = other DNA
ccccctcaatataagaa

SEQ ID NO: 68 - DNA, 18 bp, synthetic construct
  misc_feature 1..18: note = Synthetic oligonucleotide
  source 1..18: mol_type = other DNA
aaccaaccctattcctgg

SEQ ID NO: 69 - DNA, 16 bp, synthetic construct
  misc_feature 1..16: note = Synthetic oligonucleotide
  source 1..16: mol_type = other DNA
accaaccctattcctg

SEQ ID NO: 70 - DNA, 17 bp, synthetic construct
  misc_feature 1..17: note = Synthetic oligonucleotide
  source 1..17: mol_type = other DNA
aaccaaccctattcctg

SEQ ID NO: 71 - DNA, 18 bp, synthetic construct
  misc_feature 1..18: note = Synthetic oligonucleotide
  source 1..18: mol_type = other DNA
aaaaccaaccctattcct

SEQ ID NO: 72 - DNA, 16 bp, synthetic construct
  misc_feature 1..16: note = Synthetic oligonucleotide
  source 1..16: mol_type = other DNA
aaaccaaccctattcc

SEQ ID NO: 73 - DNA, 17 bp, synthetic construct
  misc_feature 1..17: note = Synthetic oligonucleotide
  source 1..17: mol_type = other DNA
ggtagtaagggcacacc

SEQ ID NO: 74 - DNA, 20 bp, synthetic construct
  misc_feature 1..20: note = Synthetic oligonucleotide
  source 1..20: mol_type = other DNA
gacaggtagtaagggcacac

SEQ ID NO: 75 - DNA, 15 bp, synthetic construct
  misc_feature 1..15: note = Synthetic oligonucleotide
  source 1..15: mol_type = other DNA
gtagtaagggcacac

SEQ ID NO: 76 - DNA, 19 bp, synthetic construct
  misc_feature 1..19: note = Synthetic oligonucleotide
  source 1..19: mol_type = other DNA
gacaggtagtaagggcaca

SEQ ID NO: 77 - DNA, 18 bp, synthetic construct
  misc_feature 1..18: note = Synthetic oligonucleotide
  source 1..18: mol_type = other DNA
acaggtagtaagggcaca

SEQ ID NO: 78 - DNA, 17 bp, synthetic construct
  misc_feature 1..17: note = Synthetic oligonucleotide
  source 1..17: mol_type = other DNA
gacaggtagtaagggca

SEQ ID NO: 79 - DNA, 18 bp, synthetic construct
  misc_feature 1..18: note = Synthetic oligonucleotide
  source 1..18: mol_type = other DNA
cgggacaggtagtaaggg

SEQ ID NO: 80 - DNA, 18 bp, synthetic construct
  misc_feature 1..18: note = Synthetic oligonucleotide
  source 1..18: mol_type = other DNA
catccaacaagtaattgt

SEQ ID NO: 81 - DNA, 20 bp, synthetic construct
  misc_feature 1..20: note = Synthetic oligonucleotide
  source 1..20: mol_type = other DNA
ggcatccaacaagtaattgt

SEQ ID NO: 82 - DNA, 19 bp, synthetic construct
  misc_feature 1..19: note = Synthetic oligonucleotide
  source 1..19: mol_type = other DNA
ggcatccaacaagtaattg

SEQ ID NO: 83 - DNA, 17 bp, synthetic construct
  misc_feature 1..17: note = Synthetic oligonucleotide
  source 1..17: mol_type = other DNA
ggcatccaacaagtaat

SEQ ID NO: 84 - DNA, 17 bp, synthetic construct
  misc_feature 1..17: note = Synthetic oligonucleotide
  source 1..17: mol_type = other DNA
cgtgaaggagagaacct

SEQ ID NO: 85 - DNA, 17 bp, synthetic construct
  misc_feature 1..17: note = Synthetic oligonucleotide
  source 1..17: mol_type = other DNA
acgtgaaggagagaacc

SEQ ID NO: 86 - DNA, 18 bp, synthetic construct
  misc_feature 1..18: note = Synthetic oligonucleotide
  source 1..18: mol_type = other DNA
gacgtgaaggagagaacc

SEQ ID NO: 87 - DNA, 17 bp, synthetic construct
  misc_feature 1..17: note = Synthetic oligonucleotide
  source 1..17: mol_type = other DNA
gacgtgaaggagagaac

SEQ ID NO: 88 - DNA, 16 bp, synthetic construct
  misc_feature 1..16: note = Synthetic oligonucleotide
  source 1..16: mol_type = other DNA
cagtcttgctatgcct

SEQ ID NO: 89 - DNA, 17 bp, synthetic construct
  misc_feature 1..17: note = Synthetic oligonucleotide
  source 1..17: mol_type = other DNA
ctagaatcaaagctcca

SEQ ID NO: 90 - DNA, 15 bp, synthetic construct
  misc_feature 1..15: note = Synthetic oligonucleotide
  source 1..15: mol_type = other DNA
acatcgcacttgggc

SEQ ID NO: 91 - DNA, 16 bp, synthetic construct
  misc_feature 1..16: note = Synthetic oligonucleotide
  source 1..16: mol_type = other DNA
cacggcaaacctcacc

SEQ ID NO: 92 - DNA, 18 bp, synthetic construct
  misc_feature 1..18: note = Synthetic oligonucleotide
  source 1..18: mol_type = other DNA
aaccacggcaaacctcac

SEQ ID NO: 93 - DNA, 17 bp, synthetic construct
  misc_feature 1..17: note = Synthetic oligonucleotide
  source 1..17: mol_type = other DNA
caaagcaccgagtcacc

SEQ ID NO: 94 - DNA, 17 bp, synthetic construct
  misc_feature 1..17: note = Synthetic oligonucleotide
  source 1..17: mol_type = other DNA
tcaaagcaccgagtcac

SEQ ID NO: 95 - DNA, 16 bp, synthetic construct
  misc_feature 1..16: note = Synthetic oligonucleotide
  source 1..16: mol_type = other DNA
ctggttgggttagaag

SEQ ID NO: 96 - DNA, 18 bp, synthetic construct
  misc_feature 1..18: note = Synthetic oligonucleotide
  source 1..18: mol_type = other DNA
tataacttttagtttagc

SEQ ID NO: 97 - DNA, 18 bp, synthetic construct
  misc_feature 1..18: note = Synthetic oligonucleotide
  source 1..18: mol_type = other DNA
ttcctttctataactttt

SEQ ID NO: 98 - DNA, 19 bp, synthetic construct
  misc_feature 1..19: note = Synthetic oligonucleotide
  source 1..19: mol_type = other DNA
gttcctttctataactttt

SEQ ID NO: 99 - DNA, 18 bp, synthetic construct
  misc_feature 1..18: note = Synthetic oligonucleotide
  source 1..18: mol_type = other DNA
gttcctttctataacttt

SEQ ID NO: 100 - DNA, 20 bp, synthetic construct
  misc_feature 1..20: note = Synthetic oligonucleotide
  source 1..20: mol_type = other DNA
atgttcctttctataacttt

SEQ ID NO: 101 - DNA, 19 bp, synthetic construct
  misc_feature 1..19: note = Synthetic oligonucleotide
  source 1..19: mol_type = other DNA
atgttcctttctataactt

SEQ ID NO: 102 - DNA, 18 bp, synthetic construct
  misc_feature 1..18: note = Synthetic oligonucleotide
  source 1..18: mol_type = other DNA
atgttcctttctataact

SEQ ID NO: 103 - DNA, 16 bp, synthetic construct
  misc_feature 1..16: note = Synthetic oligonucleotide
  source 1..16: mol_type = other DNA
gctttaatctgccttc

SEQ ID NO: 104 - DNA, 17 bp, synthetic construct
  misc_feature 1..17: note = Synthetic oligonucleotide
  source 1..17: mol_type = other DNA
ccgtggctttaatctgc

SEQ ID NO: 105 - DNA, 16 bp, synthetic construct
  misc_feature 1..16: note = Synthetic oligonucleotide
  source 1..16: mol_type = other DNA
ccgtggctttaatctg

SEQ ID NO: 106 - DNA, 17 bp, synthetic construct
  misc_feature 1..17: note = Synthetic oligonucleotide
  source 1..17: mol_type = other DNA
caaaaaacagtaggtcc

SEQ ID NO: 107 - DNA, 18 bp, synthetic construct
  misc_feature 1..18: note = Synthetic oligonucleotide
  source 1..18: mol_type = other DNA
gaataaaacagtaggtcc

SEQ ID NO: 108 - DNA, 19 bp, synthetic construct
  misc_feature 1..19: note = Synthetic oligonucleotide
  source 1..19: mol_type = other DNA
ggaataaaacagtaggtcc

SEQ ID NO: 109 - DNA, 17 bp, synthetic construct
  misc_feature 1..17: note = Synthetic oligonucleotide
  source 1..17: mol_type = other DNA
ggaataaaacagtaggt

SEQ ID NO: 110 - DNA, 16 bp, synthetic construct
  misc_feature 1..16: note = Synthetic oligonucleotide
  source 1..16: mol_type = other DNA
ggaataaaacagtagg

SEQ ID NO: 111 - DNA, 16 bp, synthetic construct
  misc_feature 1..16: note = Synthetic oligonucleotide
  source 1..16: mol_type = other DNA
cacagagtgtcatggg

SEQ ID NO: 112 - DNA, 18 bp, synthetic construct
  misc_feature 1..18: note = Synthetic oligonucleotide
  source 1..18: mol_type = other DNA
acagcatggaaaggcacg

SEQ ID NO: 113 - DNA, 17 bp, synthetic construct
  misc_feature 1..17: note = Synthetic oligonucleotide
  source 1..17: mol_type = other DNA
cagcatggaaaggcacg

SEQ ID NO: 114 - DNA, 21 bp, synthetic construct
  misc_feature 1..21: note = Synthetic oligonucleotide
  source 1..21: mol_type = other DNA
tacaggaggaagagaagggac

SEQ ID NO: 115 - DNA, 18 bp, synthetic construct
  misc_feature 1..18: note = Synthetic oligonucleotide
  source 1..18: mol_type = other DNA
acaggaggaagagaaggg

SEQ ID NO: 116 - DNA, 18 bp, synthetic construct
  misc_feature 1..18: note = Synthetic oligonucleotide
  source 1..18: mol_type = other DNA
tctacaggaggaagagaa

SEQ ID NO: 117 - DNA, 17 bp, synthetic construct
  misc_feature 1..17: note = Synthetic oligonucleotide
  source 1..17: mol_type = other DNA
tctacaggaggaagaga

SEQ ID NO: 118 - DNA, 17 bp, synthetic construct
  misc_feature 1..17: note = Synthetic oligonucleotide
  source 1..17: mol_type = other DNA
cttcgtcaaagatcacg

SEQ ID NO: 119 - DNA, 18 bp, synthetic construct
  misc_feature 1..18: note = Synthetic oligonucleotide
  source 1..18: mol_type = other DNA
gcttcgtcaaagatcacg

SEQ ID NO: 120 - DNA, 17 bp, synthetic construct
  misc_feature 1..17: note = Synthetic oligonucleotide
  source 1..17: mol_type = other DNA
gcttcgtcaaagatcac

SEQ ID NO: 121 - DNA, 15 bp, synthetic construct
  misc_feature 1..15: note = Synthetic oligonucleotide
  source 1..15: mol_type = other DNA
ccagaaaggtttgcg

SEQ ID NO: 122 - DNA, 16 bp, synthetic construct
  misc_feature 1..16: note = Synthetic oligonucleotide
  source 1..16: mol_type = other DNA
tccagaaaggtttgcg

SEQ ID NO: 123 - DNA, 17 bp, synthetic construct
  misc_feature 1..17: note = Synthetic oligonucleotide
  source 1..17: mol_type = other DNA
cagaggcatcggatcag

SEQ ID NO: 124 - DNA, 16 bp, synthetic construct
  misc_feature 1..16: note = Synthetic oligonucleotide
  source 1..16: mol_type = other DNA
cagaggcatcggatca

SEQ ID NO: 125 - DNA, 17 bp, synthetic construct
  misc_feature 1..17: note = Synthetic oligonucleotide
  source 1..17: mol_type = other DNA
agcagaggcatcggatc

SEQ ID NO: 126 - DNA, 17 bp, synthetic construct
  misc_feature 1..17: note = Synthetic oligonucleotide
  source 1..17: mol_type = other DNA
attcttcacacatcttc

SEQ ID NO: 127 - DNA, 15 bp, synthetic construct
  misc_feature 1..15: note = Synthetic oligonucleotide
  source 1..15: mol_type = other DNA
ctatgaacgcacctg

SEQ ID NO: 128 - DNA, 17 bp, synthetic construct
  misc_feature 1..17: note = Synthetic oligonucleotide
  source 1..17: mol_type = other DNA
ggctatgaacgcacctg

SEQ ID NO: 129 - DNA, 17 bp, synthetic construct
  misc_feature 1..17: note = Synthetic oligonucleotide
  source 1..17: mol_type = other DNA
gctgggagaagacatag

SEQ ID NO: 130 - DNA, 19 bp, synthetic construct
  misc_feature 1..19: note = Synthetic oligonucleotide
  source 1..19: mol_type = other DNA
caaaatgcccttacagtga

SEQ ID NO: 131 - DNA, 17 bp, synthetic construct
  misc_feature 1..17: note = Synthetic oligonucleotide
  source 1..17: mol_type = other DNA
caaaatgcccttacagt

SEQ ID NO: 132 - DNA, 21 bp, synthetic construct
  misc_feature 1..21: note = Synthetic oligonucleotide
  source 1..21: mol_type = other DNA
tgtgcgattttaaaggaaaat

SEQ ID NO: 133 - DNA, 21 bp, synthetic construct
  misc_feature 1..21: note = Synthetic oligonucleotide
  source 1..21: mol_type = other DNA
catgtgcgattttaaaggaaa

SEQ ID NO: 134 - DNA, 18 bp, synthetic construct
  misc_feature 1..18: note = Synthetic oligonucleotide
  source 1..18: mol_type = other DNA
tgtgcgattttaaaggaa

SEQ ID NO: 135 - DNA, 19 bp, synthetic construct
  misc_feature 1..19: note = Synthetic oligonucleotide
  source 1..19: mol_type = other DNA
catgtgcgattttaaagga

SEQ ID NO: 136 - DNA, 18 bp, synthetic construct
  misc_feature 1..18: note = Synthetic oligonucleotide
  source 1..18: mol_type = other DNA
atgtgcgattttaaagga

SEQ ID NO: 137 - DNA, 21 bp, synthetic construct
  misc_feature 1..21: note = Synthetic oligonucleotide
  source 1..21: mol_type = other DNA
accctgtcacttaaatatatg

SEQ ID NO: 138 - DNA, 17 bp, synthetic construct
  misc_feature 1..17: note = Synthetic oligonucleotide
  source 1..17: mol_type = other DNA
gagggaggtggagcgtt

SEQ ID NO: 139 - DNA, 17 bp, synthetic construct
  misc_feature 1..17: note = Synthetic oligonucleotide
  source 1..17: mol_type = other DNA
ctgaagagtggagaagg

SEQ ID NO: 140 - DNA, 20 bp, synthetic construct
  misc_feature 1..20: note = Synthetic oligonucleotide
  source 1..20: mol_type = other DNA
caataaataaagtgtgagga

SEQ ID NO: 141 - DNA, 18 bp, synthetic construct
  misc_feature 1..18: note = Synthetic oligonucleotide
  source 1..18: mol_type = other DNA
caacccagtaaccatgac

SEQ ID NO: 142 - DNA, 17 bp, synthetic construct
  misc_feature 1..17: note = Synthetic oligonucleotide
  source 1..17: mol_type = other DNA
caacccagtaaccatga

SEQ ID NO: 143 - DNA, 19 bp, synthetic construct
  misc_feature 1..19: note = Synthetic oligonucleotide
  source 1..19: mol_type = other DNA
accaacccagtaaccatga

SEQ ID NO: 144 - DNA, 16 bp, synthetic construct
  misc_feature 1..16: note = Synthetic oligonucleotide
  source 1..16: mol_type = other DNA
gagcaggtgttttatc

SEQ ID NO: 145 - DNA, 17 bp, synthetic construct
  misc_feature 1..17: note = Synthetic oligonucleotide
  source 1..17: mol_type = other DNA
ggtcgaggaggtgtcac

SEQ ID NO: 146 - DNA, 16 bp, synthetic construct
  misc_feature 1..16: note = Synthetic oligonucleotide
  source 1..16: mol_type = other DNA
gtcgaggaggtgtcac

SEQ ID NO: 147 - DNA, 16 bp, synthetic construct
  misc_feature 1..16: note = Synthetic oligonucleotide
  source 1..16: mol_type = other DNA
ggtcgaggaggtgtca

SEQ ID NO: 148 - DNA, 15 bp, synthetic construct
  misc_feature 1..15: note = Synthetic oligonucleotide
  source 1..15: mol_type = other DNA
ggtcgaggaggtgtc

SEQ ID NO: 149 - DNA, 17 bp, synthetic construct
  misc_feature 1..17: note = Synthetic oligonucleotide
  source 1..17: mol_type = other DNA
ccaggtctcaaaaaggg

SEQ ID NO: 150 - DNA, 15 bp, synthetic construct
  misc_feature 1..15: note = Synthetic oligonucleotide
  source 1..15: mol_type = other DNA
attacgctgaggaca

SEQ ID NO: 151 - DNA, 15 bp, synthetic construct
  misc_feature 1..15: note = Synthetic oligonucleotide
  source 1..15: mol_type = other DNA
cattacgctgaggac

SEQ ID NO: 152 - DNA, 14 bp, synthetic construct
  misc_feature 1..14: note = Synthetic oligonucleotide
  source 1..14: mol_type = other DNA
cttgagcattacgc

SEQ ID NO: 153 - DNA, 17 bp, synthetic construct
  misc_feature 1..17: note = Synthetic oligonucleotide
  source 1..17: mol_type = other DNA
cgaggagaagaaggcag

SEQ ID NO: 154 - DNA, 19 bp, synthetic construct
  misc_feature 1..19: note = Synthetic oligonucleotide
  source 1..19: mol_type = other DNA
ccttggtctgaaacgtgat

SEQ ID NO: 155 - DNA, 15 bp, synthetic construct
  misc_feature 1..15: note = Synthetic oligonucleotide
  source 1..15: mol_type = other DNA
ctaacgcctccacgc

SEQ ID NO: 156 - DNA, 15 bp, synthetic construct
  misc_feature 1..15: note = Synthetic oligonucleotide
  source 1..15: mol_type = other DNA
ggacaggctctacgg

SEQ ID NO: 157 - DNA, 20 bp, synthetic construct
  misc_feature 1..20: note = Synthetic oligonucleotide
  source 1..20: mol_type = other DNA
actaatacagcaggagaagg

SEQ ID NO: 158 - DNA, 21 bp, synthetic construct
  misc_feature 1..21: note = Synthetic oligonucleotide
  source 1..21: mol_type = other DNA
aactaatacagcaggagaagg

SEQ ID NO: 159 - DNA, 21 bp, synthetic construct
  misc_feature 1..21: note = Synthetic oligonucleotide
  source 1..21: mol_type = other DNA
taactaatacagcaggagaag

SEQ ID NO: 160 - DNA, 16 bp, synthetic construct
  misc_feature 1..16: note = Synthetic oligonucleotide
  source 1..16: mol_type = other DNA
ttgaagagccaaccac

SEQ ID NO: 161 - DNA, 17 bp, synthetic construct
  misc_feature 1..17: note = Synthetic oligonucleotide
  source 1..17: mol_type = other DNA
ccattttcactgtcaag

SEQ ID NO: 162 - DNA, 17 bp, synthetic construct
  misc_feature 1..17: note = Synthetic oligonucleotide
  source 1..17: mol_type = other DNA
gccattttcactgtcaa

SEQ ID NO: 163 - DNA, 16 bp, synthetic construct
  misc_feature 1..16: note = Synthetic oligonucleotide
  source 1..16: mol_type = other DNA
agaaatgcggagaagc

SEQ ID NO: 164 - DNA, 20 bp, synthetic construct
  misc_feature 1..20: note = Synthetic oligonucleotide
  source 1..20: mol_type = other DNA
aaatggaaaaaatgaccagc

SEQ ID NO: 165 - DNA, 20 bp, synthetic construct
  misc_feature 1..20: note = Synthetic oligonucleotide
  source 1..20: mol_type = other DNA
aggacttacgacaaaaccac

SEQ ID NO: 166 - DNA, 18 bp, synthetic construct
  misc_feature 1..18: note = Synthetic oligonucleotide
  source 1..18: mol_type = other DNA
ggacttacgacaaaacca

SEQ ID NO: 167 - DNA, 17 bp, synthetic construct
  misc_feature 1..17: note = Synthetic oligonucleotide
  source 1..17: mol_type = other DNA
gacttacgacaaaacca

SEQ ID NO: 168 - DNA, 18 bp, synthetic construct
  misc_feature 1..18: note = Synthetic oligonucleotide
  source 1..18: mol_type = other DNA
acaccaggacttacgaca

SEQ ID NO: 169 - DNA, 17 bp, synthetic construct
  misc_feature 1..17: note = Synthetic oligonucleotide
  source 1..17: mol_type = other DNA
tagaaattcaacatggc

SEQ ID NO: 170 - DNA, 18 bp, synthetic construct
  misc_feature 1..18: note = Synthetic oligonucleotide
  source 1..18: mol_type = other DNA
ctagaaattcaacatggc

SEQ ID NO: 171 - DNA, 14 bp, synthetic construct
  misc_feature 1..14: note = Synthetic oligonucleotide
  source 1..14: mol_type = other DNA
gtcatcggttcacc

SEQ ID NO: 172 - DNA, 14 bp, synthetic construct
  misc_feature 1..14: note = Synthetic oligonucleotide
  source 1..14: mol_type = other DNA
actcgaagacgcca

SEQ ID NO: 173 - DNA, 14 bp, synthetic construct
  misc_feature 1..14: note = Synthetic oligonucleotide
  source 1..14: mol_type = other DNA
gactcgaagacgcc

SEQ ID NO: 174 - DNA, 17 bp, synthetic construct
  misc_feature 1..17: note = Synthetic oligonucleotide
  source 1..17: mol_type = other DNA
ggcacaagcagaacgac

SEQ ID NO: 175 - DNA, 18 bp, synthetic construct
  misc_feature 1..18: note = Synthetic oligonucleotide
  source 1..18: mol_type = other DNA
agtcagaacaaaggaggc

SEQ ID NO: 176 - DNA, 18 bp, synthetic construct
  misc_feature 1..18: note = Synthetic oligonucleotide
  source 1..18: mol_type = other DNA
gaagtcagaacaaaggag

SEQ ID NO: 177 - DNA, 19 bp, synthetic construct
  misc_feature 1..19: note = Synthetic oligonucleotide
  source 1..19: mol_type = other DNA
gcagaagtcagaacaaagg

SEQ ID NO: 178 - DNA, 19 bp, synthetic construct
  misc_feature 1..19: note = Synthetic oligonucleotide
  source 1..19: mol_type = other DNA
gtgcagaagtcagaacaaa

SEQ ID NO: 179 - DNA, 18 bp, synthetic construct
  misc_feature 1..18: note = Synthetic oligonucleotide
  source 1..18: mol_type = other DNA
gtgcagaagtcagaacaa

SEQ ID NO: 180 - DNA, 18 bp, synthetic construct
  misc_feature 1..18: note = Synthetic oligonucleotide
  source 1..18: mol_type = other DNA
aaggatgagggagcggac

SEQ ID NO: 181 - DNA, 16 bp, synthetic construct
  misc_feature 1..16: note = Synthetic oligonucleotide
  source 1..16: mol_type = other DNA
gtaaggatgagggagc

SEQ ID NO: 182 - DNA, 17 bp, synthetic construct
  misc_feature 1..17: note = Synthetic oligonucleotide
  source 1..17: mol_type = other DNA
tggtaaggatgagggag

SEQ ID NO: 183 - DNA, 16 bp, synthetic construct
  misc_feature 1..16: note = Synthetic oligonucleotide
  source 1..16: mol_type = other DNA
cgtacatctgcatctc

SEQ ID NO: 184 - DNA, 21 bp, synthetic construct
  misc_feature 1..21: note = Synthetic oligonucleotide
  source 1..21: mol_type = other DNA
tgtaagataagaggcaacact

SEQ ID NO: 185 - DNA, 21 bp, synthetic construct
  misc_feature 1..21: note = Synthetic oligonucleotide
  source 1..21: mol_type = other DNA
ttgtaagataagaggcaacac

SEQ ID NO: 186 - DNA, 20 bp, synthetic construct
  misc_feature 1..20: note = Synthetic oligonucleotide
  source 1..20: mol_type = other DNA
ttgtaagataagaggcaaca

SEQ ID NO: 187 - DNA, 21 bp, synthetic construct
  misc_feature 1..21: note = Synthetic oligonucleotide
  source 1..21: mol_type = other DNA
tttgtaagataagaggcaaca

SEQ ID NO: 188 - DNA, 17 bp, synthetic construct
  misc_feature 1..17: note = Synthetic oligonucleotide
  source 1..17: mol_type = other DNA
tgtaagataagaggcaa

SEQ ID NO: 189 - DNA, 17 bp, synthetic construct
  misc_feature 1..17: note = Synthetic oligonucleotide
  source 1..17: mol_type = other DNA
ctggaaggaaagttggt

SEQ ID NO: 190 - DNA, 19 bp, synthetic construct
  misc_feature 1..19: note = Synthetic oligonucleotide
  source 1..19: mol_type = other DNA
atagtaagcactgatggtc

SEQ ID NO: 191 - DNA, 16 bp, synthetic construct
  misc_feature 1..16: note = Synthetic oligonucleotide
  source 1..16: mol_type = other DNA
tagtaagcactgatgg

SEQ ID NO: 192 - DNA, 17 bp, synthetic construct
  misc_feature 1..17: note = Synthetic oligonucleotide
  source 1..17: mol_type = other DNA
catagtaagcactgatg

SEQ ID NO: 193 - DNA, 16 bp, synthetic construct
  misc_feature 1..16: note = Synthetic oligonucleotide
  source 1..16: mol_type = other DNA
ctgtaactcacctggc

SEQ ID NO: 194 - DNA, 15 bp, synthetic construct
  misc_feature 1..15: note = Synthetic oligonucleotide
  source 1..15: mol_type = other DNA
cggatcactcgcccg

SEQ ID NO: 195 - DNA, 17 bp, synthetic construct
  misc_feature 1..17: note = Synthetic oligonucleotide
  source 1..17: mol_type = other DNA
acacaggctactctcgg

SEQ ID NO: 196 - DNA, 16 bp, synthetic construct
  misc_feature 1..16: note = Synthetic oligonucleotide
  source 1..16: mol_type = other DNA
acacaggctactctcg

SEQ ID NO: 197 - DNA, 17 bp, synthetic construct
  misc_feature 1..17: note = Synthetic oligonucleotide
  source 1..17: mol_type = other DNA
ccacacacaggctactc

SEQ ID NO: 198 - DNA, 18 bp, synthetic construct
  misc_feature 1..18: note = Synthetic oligonucleotide
  source 1..18: mol_type = other DNA
atactccacacacaggct

SEQ ID NO: 199 - DNA, 17 bp, synthetic construct
  misc_feature 1..17: note = Synthetic oligonucleotide
  source 1..17: mol_type = other DNA
atactccacacacaggc

SEQ ID NO: 200 - DNA, 19 bp, synthetic construct
  misc_feature 1..19: note = Synthetic oligonucleotide
  source 1..19: mol_type = other DNA
gctcatactccacacacag

SEQ ID NO: 201 - DNA, 17 bp, synthetic construct
  misc_feature 1..17: note = Synthetic oligonucleotide
  source 1..17: mol_type = other DNA
tcatactccacacacag

SEQ ID NO: 202 - DNA, 18 bp, synthetic construct
  misc_feature 1..18: note = Synthetic oligonucleotide
  source 1..18: mol_type = other DNA
gctcatactccacacaca

SEQ ID NO: 203 - DNA, 18 bp, synthetic construct
  misc_feature 1..18: note = Synthetic oligonucleotide
  source 1..18: mol_type = other DNA
tgctcatactccacacac

SEQ ID NO: 204 - DNA, 19 bp, synthetic construct
  misc_feature 1..19: note = Synthetic oligonucleotide
  source 1..19: mol_type = other DNA
agcaggaagcagggagaaa

SEQ ID NO: 205 - DNA, 15 bp, synthetic construct
  misc_feature 1..15: note = Synthetic oligonucleotide
  source 1..15: mol_type = other DNA
tccgaccacagcgag

SEQ ID NO: 206 - DNA, 18 bp, synthetic construct
  misc_feature 1..18: note = Synthetic oligonucleotide
  source 1..18: mol_type = other DNA
cagaagccaagggacatg

SEQ ID NO: 207 - DNA, 17 bp, synthetic construct
  misc_feature 1..17: note = Synthetic oligonucleotide
  source 1..17: mol_type = other DNA
cagaagccaagggacat

SEQ ID NO: 208 - DNA, 19 bp, synthetic construct
  misc_feature 1..19: note = Synthetic oligonucleotide
  source 1..19: mol_type = other DNA
ccagaccaacacggaaacg

SEQ ID NO: 209 - DNA, 18 bp, synthetic construct
  misc_feature 1..18: note = Synthetic oligonucleotide
  source 1..18: mol_type = other DNA
ccagaccaacacggaaac

SEQ ID NO: 210 - DNA, 18 bp, synthetic construct
  misc_feature 1..18: note = Synthetic oligonucleotide
  source 1..18: mol_type = other DNA
gaatgggcaaagggtaga

SEQ ID NO: 211 - DNA, 17 bp, synthetic construct
  misc_feature 1..17: note = Synthetic oligonucleotide
  source 1..17: mol_type = other DNA
aatgggcaaagggtaga

SEQ ID NO: 212 - DNA, 17 bp, synthetic construct
  misc_feature 1..17: note = Synthetic oligonucleotide
  source 1..17: mol_type = other DNA
gaatgggcaaagggtag

SEQ ID NO: 213 - DNA, 17 bp, synthetic construct
  misc_feature 1..17: note = Synthetic oligonucleotide
  source 1..17: mol_type = other DNA
gaacccttgtagtcctg

SEQ ID NO: 214 - DNA, 15 bp, synthetic construct
  misc_feature 1..15: note = Synthetic oligonucleotide
  source 1..15: mol_type = other DNA
aacccttgtagtcct

SEQ ID NO: 215 - DNA, 15 bp, synthetic construct
  misc_feature 1..15: note = Synthetic oligonucleotide
  source 1..15: mol_type = other DNA
ggaacccttgtagtc

SEQ ID NO: 216 - DNA, 18 bp, synthetic construct
  misc_feature 1..18: note = Synthetic oligonucleotide
  source 1..18: mol_type = other DNA
atcggaacccttgtagtc

SEQ ID NO: 217 - DNA, 19 bp, synthetic construct
  misc_feature 1..19: note = Synthetic oligonucleotide
  source 1..19: mol_type = other DNA
catcggaacccttgtagtc

SEQ ID NO: 218 - DNA, 18 bp, synthetic construct
  misc_feature 1..18: note = Synthetic oligonucleotide
  source 1..18: mol_type = other DNA
catcggaacccttgtagt

SEQ ID NO: 219 - DNA, 20 bp, synthetic construct
  misc_feature 1..20: note = Synthetic oligonucleotide
  source 1..20: mol_type = other DNA
gatacagacctcctcaaact

SEQ ID NO: 220 - DNA, 19 bp, synthetic construct
  misc_feature 1..19: note = Synthetic oligonucleotide
  source 1..19: mol_type = other DNA
gatacagacctcctcaaac

SEQ ID NO: 221 - DNA, 18 bp, synthetic construct
  misc_feature 1..18: note = Synthetic oligonucleotide
  source 1..18: mol_type = other DNA
gatacagacctcctcaaa

SEQ ID NO: 222 - DNA, 17 bp, synthetic construct
  misc_feature 1..17: note = Synthetic oligonucleotide
  source 1..17: mol_type = other DNA
gatacagacctcctcaa

SEQ ID NO: 223 - DNA, 16 bp, synthetic construct
  misc_feature 1..16: note = Synthetic oligonucleotide
  source 1..16: mol_type = other DNA
gccccatttaccagtg

SEQ ID NO: 224 - DNA, 16 bp, synthetic construct
  misc_feature 1..16: note = Synthetic oligonucleotide
  source 1..16: mol_type = other DNA
cccaacaagtgatgct

SEQ ID NO: 225 - DNA, 15 bp, synthetic construct
  misc_feature 1..15: note = Synthetic oligonucleotide
  source 1..15: mol_type = other DNA
cccaacaagtgatgc

SEQ ID NO: 226 - DNA, 17 bp, synthetic construct
  misc_feature 1..17: note = Synthetic oligonucleotide
  source 1..17: mol_type = other DNA
gtaccaagcccagaagg

SEQ ID NO: 227 - DNA, 16 bp, synthetic construct
  misc_feature 1..16: note = Synthetic oligonucleotide
  source 1..16: mol_type = other DNA
gtaccaagcccagaag

SEQ ID NO: 228 - DNA, 17 bp, synthetic construct
  misc_feature 1..17: note = Synthetic oligonucleotide
  source 1..17: mol_type = other DNA
ttcctgatgaagagatg

SEQ ID NO: 229 - DNA, 16 bp, synthetic construct
  misc_feature 1..16: note = Synthetic oligonucleotide
  source 1..16: mol_type = other DNA
tcctgatgaagagatg

SEQ ID NO: 230 - DNA, 15 bp, synthetic construct
  misc_feature 1..15: note = Synthetic oligonucleotide
  source 1..15: mol_type = other DNA
tgggagtagcatggc

SEQ ID NO: 231 - DNA, 17 bp, synthetic construct
  misc_feature 1..17: note = Synthetic oligonucleotide
  source 1..17: mol_type = other DNA
tgtgggagtagcatggc

SEQ ID NO: 232 - DNA, 16 bp, synthetic construct
  misc_feature 1..16: note = Synthetic oligonucleotide
  source 1..16: mol_type = other DNA
gtgggagtagcatggc

SEQ ID NO: 233 - DNA, 17 bp, synthetic construct
  misc_feature 1..17: note = Synthetic oligonucleotide
  source 1..17: mol_type = other DNA
aaacatgctgaaccctg

SEQ ID NO: 234 - DNA, 18 bp, synthetic construct
  misc_feature 1..18: note = Synthetic oligonucleotide
  source 1..18: mol_type = other DNA
acaaacatgctgaaccct

SEQ ID NO: 235 - DNA, 18 bp, synthetic construct
  misc_feature 1..18: note = Synthetic oligonucleotide
  source 1..18: mol_type = other DNA
cacaaacatgctgaaccc

SEQ ID NO: 236 - DNA, 17 bp, synthetic construct
  misc_feature 1..17: note = Synthetic oligonucleotide
  source 1..17: mol_type = other DNA
cacaaacatgctgaacc

SEQ ID NO: 237 - DNA, 17 bp, synthetic construct
  misc_feature 1..17: note = Synthetic oligonucleotide
  source 1..17: mol_type = other DNA
tggacgcacaaacatgc

SEQ ID NO: 238 - DNA, 21 bp, synthetic construct
  misc_feature 1..21: note = Primer
  source 1..21: mol_type = other DNA
ggagtgtggattcgcactcct

SEQ ID NO: 239 - DNA, 20 bp, synthetic construct
  misc_feature 1..20: note = Primer
  source 1..20: mol_type = other DNA
agattgagatcttctgcgac

SEQ ID NO: 240 - DNA, 27 bp, synthetic construct
  misc_feature 1..27: note = probe
  source 1..27: mol_type = other DNA
aggcaggtcccctagaagaagaactcc

SEQ ID NO: 241 - DNA, 21 bp, synthetic construct
  misc_feature 1..21: note = primer
  source 1..21: mol_type = other DNA
ccatcctggacattgaggact

SEQ ID NO: 242 - DNA, 21 bp, synthetic construct
  misc_feature 1..21: note = primer
  source 1..21: mol_type = other DNA
caggttccgggacaggtagta

SEQ ID NO: 243 - DNA, 21 bp, synthetic construct
  misc_feature 1..21: note = primer
  source 1..21: mol_type = other DNA
cgtctgtgccttctcatctgc

SEQ ID NO: 244 - DNA, 20 bp, synthetic construct
  misc_feature 1..20: note = primer
  source 1..20: mol_type = other DNA
gcacagcttggaggcttgaa

SEQ ID NO: 245 - DNA, 20 bp, synthetic construct
  misc_feature 1..20: note = primer
  source 1..20: mol_type = other DNA
ccgtctgaactatcctgccc

SEQ ID NO: 246 - DNA, 20 bp, synthetic construct
  misc_feature 1..20: note = primer
  source 1..20: mol_type = other DNA
gccgtagtcggtgtactcgt

(19) United States
(12) Patent Application Publication
(10) Pub. No.: US 2026/0002161 A1
(43) Pub. Date: Jan. 1, 2026

(54) OLIGONUCLEOTIDES FOR MODULATING RTEL1 EXPRESSION

(71) Applicant: Hoffmann-La Roche Inc., Little Falls, NJ (US)

(72) Inventors: Marco Berrera, Flueh (CH); Josephine Felber, Basel (CH); Jean-Christophe Hoflack, Saint-Louis (FR); Susanne Kammler, Holte (DK); Tony Kam-Thong, Basel (CH); Brian Leonard, Riehen (CH); Lykke Pedersen, Copenhagen NV (DK); Philipp Tropberger, Loerrach (DE); Miriam Triyatni, Basel (CH); Daniel Jeremy Turley, Basel (CH); Angelina Wallier, Flueh (CH); Jitao David Zhang, Riehen (CH)

(21) Appl. No.: 19/320,635

(22) Filed: Sep. 5, 2025

Related U.S. Application Data
(62) Division of application No. 17/147,797, filed on Jan. 13, 2021.
Application No. 17/147,797 is a continuation of application No. PCT/EP2019/068639, filed on Jul. 11, 2019.

(30) Foreign Application Priority Data
Jul. 13, 2018 (EP) 18183477.1

(51) Int. Cl. C12N 15/113; A61P 31/20

(52) U.S. Cl. CPC C12N 15/1137; A61P 31/20; C12N 2310/11; C12N 2310/315; C12N 2310/3231; C12N 2310/3341; C12N 2310/341; C12N 2310/351

(57) ABSTRACT

The present invention relates to a RTEL1 inhibitor for use in treatment of an HBV infection, in particular a chronic HBV infection. The invention in particular relates to the use of RTEL1 inhibitors for destabilizing cccDNA, such as HBV cccDNA. The invention also relates to antisense oligonucleotides which are complementary to RTEL1 and capable of reducing a RTEL1 mRNA. Also comprised in the present invention is a pharmaceutical composition and its use in the treatment and/or prevention of a HBV infection.

### SEQUENCE LISTING

The instant application contains a Sequence Listing which has been submitted electronically in XML format and is hereby incorporated by reference in its entirety. Said XML copy, created on Sep. 5, 2025, is named 51551-005004_Sequence_Listing_9_5_25 and is 382,661bytes in size.

### FIELD OF INVENTION

The present invention relates to RTEL1 inhibitors, such as oligonucleotides (oligomers) that are complementary to RTEL1, leading to modulation of the expression of RTEL1 or modulation of RTEL1 activity. The invention in particular relates to the use of RTEL1 targeting nucleic acid molecules for use in treating and/or preventing a hepatitis B virus (HBV) infection, in particular a chronic HBV infection. The invention in particular relates to the use of RTEL1 inhibitors for destabilizing cccDNA, such as HBV cccDNA. Also comprised in the present invention is a pharmaceutical composition and its use in the treatment and/or prevention of a HBV infection.

### BACKGROUND

Hepatitis B is an infectious disease caused by the hepatitis B virus (HBV), a small hepatotropic virus that replicates through reverse transcription. Chronic HBV infection is a key factor for severe liver diseases such as liver cirrhosis and hepatocellular carcinoma. Current treatments for chronic HBV infection are based on administration of pegylated type 1 interferons or nucleos(t)ide analogues, such as lamivudine, adefovir, entecavir, tenofovir disoproxil, and tenofovir alafenamide, which target the viral polymerase, a multifunctional reverse transcriptase. Treatment success is usually measured as loss of hepatitis B surface antigen (HBsAg). However, a complete HBsAg clearance is rarely achieved since Hepatitis B virus DNA persists in the body after infection. HBV persistence is mediated by an episomal form of the HBV genome which is stably maintained in the nucleus. This episomal form is called “covalently closed circular DNA” (cccDNA). The cccDNA serves as a template for all HBV transcripts, including pregenomic RNA (pgRNA), a viral replicative intermediate. The presence of a few copies of cccDNA might be sufficient to reinitiate a full-blown HBV infection. Current treatments for HBV do not target cccDNA. A cure of chronic HBV infection, however, would require the elimination of cccDNA (reviewed by Nassal, Gut. 2015 December;64(12):1972-84.).

Regulator of telomere elongation helicase 1 (RTEL1) encodes a DNA helicase which functions in the stability, protection and elongation of telomeres and interacts with proteins in the shelterin complex known to protect telomeres during DNA replication. Mutations in this gene have been associated with dyskeratosis congenita and Hoyerall-Hreidarsson syndrome (See for example review by Vannier et al 2014 Trends Cell Biol. Vol 24 p.416).

Located in the nucleus, RTEL1 functions as an ATP-dependent DNA helicase implicated in telomere-length regulation, DNA repair and the maintenance of genomic stability. RTEL1 Acts as an anti-recombinase to counteract toxic recombination and limit crossover during meiosis and regulates meiotic recombination and crossover homeostasis by physically dissociating strand invasion events and thereby promotes non-crossover repair by meiotic synthesis dependent strand annealing (SDSA) as well as disassembly of D loop recombination intermediates. In additional RTEL1 disassembles T loops and prevents telomere fragility by counteracting telomeric G4-DNA structures, which together ensure the dynamics and stability of the telomere.

RTEL1 has been identified in a siRNA screen as a stabilizer of HPV episomes: (Edwards et al 2013 PLoS One Vol 8, e75406). siRNA targeting RTEL1 has likewise been used to identify interactants with RTEL1 in Hoyeraal-Hreidarsson syndrome (Schertzer et al 2015 Nucleic Acid Res Vol 43 p. 1834). In addition, RTEL1 was identified as a HIV host dependency factor from a siRNA screen for essential host proteins to provide targets for inhibition HIV infection (WO 2007/094818).

To our knowledge RTEL1 has never been identified as a cccDNA dependency factor in the context of cccDNA stability and maintenance, nor have molecules inhibiting RTEL1 ever been suggested as cccDNA destabilizers for the treatment of HBV infection.

### OBJECTIVE OF THE INVENTION

The present invention shows that there is a correlation between the inhibition of RTEL1 and reduction of cccDNA in an HBV infected cell, which is relevant in the treatment of HBV infected individuals. An objective of the present invention is to identify RTEL1 inhibitors which reduce cccDNA in an HBV infected cell. Such RTEL1 inhibitors can be used in the treatment of HBV infection.

The present invention further identifies novel nucleic acid molecules, which are capable of inhibiting the expression of RTEL1 in vitro and in vivo.

### SUMMARY OF INVENTION

The present invention relates to oligonucleotides targeting a nucleic acid capable of modulating the expression of RTEL1 and to treat or prevent diseases related to the functioning of the RTEL1.

Accordingly, in a first aspect the invention provides a RTEL1 inhibitor for use in the treatment and/or prevention of Hepatitis B virus (HBV) infection. In particular, a RTEL1 inhibitor capable of reducing cccDNA and/or pre-genomic RNA (pgRNA) is useful. Such an inhibitor is advantageously selected from a nucleic acid molecule of 12 to 60 nucleotides in length, which is capable of reducing RTEL1 mRNA, such as a single stranded antisense oligonucleotide, a siRNA or a shRNA complementary to mammalian RTEL1

In a further aspect the invention relates to an oligonucleotide of 12-60 nucleotides, such as 12-30 nucleotides, comprising a contiguous nucleotides sequence of at least 10 nucleotides, in particular of 16 to 20 nucleotides, which is complementary to a mammalian RTEL1. Such an oligonucleotide is capable of inhibiting the expression of RTEL1. The oligonucleotide can be a single stranded antisense oligonucleotide or a shRNA nucleic acid molecule.

The antisense oligonucleotide can have a gapmer design. Preferably, the antisense oligonucleotide is capable of inhibiting the expression of RTEL1 by cleavage of the target nucleic acid. The cleavage is preferably achieved via nuclease recruitment.

In a further aspect, the invention provides pharmaceutical compositions comprising the antisense oligonucleotide of the invention and a pharmaceutically excipient.

In a further aspect, the invention provides methods for in vivo or in vitro method for modulation of RTEL1 expression in a target cell which is expressing RTEL1, by administering an antisense oligonucleotide or composition of the invention in an effective amount to said cell.

In a further aspect the invention provides methods for treating or preventing a disease, disorder or dysfunction associated with in vivo activity of RTEL1 comprising administering a therapeutically or prophylactically effective amount of the antisense oligonucleotide of the invention to a subject suffering from or susceptible to the disease, disorder or dysfunction.

Further aspects of the invention are conjugates of nucleic acid molecules of the invention and pharmaceutical compositions comprising the molecules of the invention. In particular conjugates targeting the liver are of interest, such as GalNAc clusters.

### BRIEF DESCRIPTION OF FIGURES

FIG. 1: Illustrates exemplary antisense oligonucleotide conjugates, where the oligonucleotide either is represented as a wavy line (A-D) or as “oligonucleotide” (E-H) or as T₂(I) and the asialoglycoprotein receptor targeting conjugate moieties are trivalent N-acetylgalactosamine moieties. Compounds A to D comprise a di-lysine brancher molecule, a PEG3 spacer and three terminal GalNAc carbohydrate moieties. In compound A and B the oligonucleotide is attached directly to the asialoglycoprotein receptor targeting conjugate moiety without a linker. In compound C and D the oligonucleotide is attached to the asialoglycoprotein receptor targeting conjugate moiety via a C6 linker. Compounds E-I comprise a commercially available trebler brancher molecule and spacers of varying length and structure and three terminal GalNAc carbohydrate moieties.

### DEFINITIONS

### HBV infection

The term “hepatitis B virus infection” or “HBV infection” is commonly known in the art and refers to an infectious disease that is caused by the hepatitis B virus (HBV) and affects the liver. A HBV infection can be an acute or a chronic infection. Chronic hepatitis B virus (CHB) infection is a global disease burden affecting 248 million individuals worldwide. Approximately 686,000 deaths annually are attributed to HBV-related end-stage liver diseases and hepatocellular carcinoma (HCC) (GBD 2013; Schweitzer et al., 2015). WHO projected that without expanded intervention, the number of people living with CHB infection will remain at the current high levels for the next 40-50 years, with a cumulative 20 million deaths occurring between 2015 and 2030 (WHO 2016). CHB infection is not a homogenous disease with singular clinical presentation. Infected individuals have progressed through several phases of CHB-associated liver disease in their life; these phases of disease are also the basis for treatment with standard of care (SOC). Current guidelines recommend treating only selected CHB-infected individuals based on three criteria—serum ALT level, HBV DNA level, and severity of liver disease (EASL, 2017). This recommendation was due to the fact that SOC i.e. nucleos(t)ide analogs (NAs) and pegylated interferon-alpha (PEG-IFN), are not curative and must be administered for long periods of time thereby increasing their safety risks. NAs effectively suppress HBV DNA replication; however, they have very limited/no effect on other viral markers. Two hallmarks of HBV infection, hepatitis B surface antigen (HBsAg) and covalently closed circular DNA (cccDNA), are the main targets of novel drugs aiming for HBV cure. In the plasma of CHB individuals, HBsAg subviral (empty) particles outnumber HBV virions by a factor of 103 to 105 (Ganem & Prince, 2014); its excess is believed to contribute to immunopathogenesis of the disease, including inability of individuals to develop neutralizing anti-HBs antibody, the serological marker observed following resolution of acute HBV infection.

### CccDNA (Covalently Closed Circular DNA)

cccDNA is the viral genetic template that resides in the nucleus of infected hepatocytes, where it gives rise to all HBV RNA transcripts needed for productive infection and is responsible for viral persistence during natural course of chronic HBV infection (Locarnini & Zoulim, 2010 Antivir Ther. 15 Suppl 3:3-14.). Acting as a viral reservoir, cccDNA is the source of viral rebound after cessation of treatment, necessitating long term, often, lifetime treatment. PEG-IFN can only be administered to a small subset of CHB due to its various side effects.

Consequently, novel therapies that can deliver a complete cure, defined by degradation or elimination of HBV cccDNA, to the majority of CHB patients are highly needed.

### Compound

Herein, the term “compound” means any molecule capable of inhibition RTEL1 expression or activity. Particular compounds of the invention are nucleic acid molecules, such as RNAi molecules or antisense oligonucleotides according to the invention or any conjugate comprising such a nucleic acid molecule. For example, herein the compound may be a nucleic acid molecule targeting RTEL1, in particular an antisense oligonucleotide or a siRNA.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers, which may be used interchangeably.

The oligonucleotides referred to in in the description and claims are generally therapeutic oligonucleotides below 70 nucleotides in length. The oligonucleotide may be or comprise a single stranded antisense oligonucleotide, or may be another oligomeric nucleic acid molecule, such as a CRISPR RNA, a siRNA, shRNA, an aptamer, or a ribozyme. Therapeutic oligonucleotide molecules are commonly made in the laboratory by solid-phase chemical synthesis followed by purification and isolation. shRNA's are however often delivered to cells using lentiviral vectors from which they are then transcribed to produce the single stranded RNA that will form a stem loop (hairpin) RNA structure that is capable of interacting with the RNA interference machinery (including the RNA-induced silencing complex (RISC)). In an embodiment of the present invention the shRNA is chemically produced shRNA molecules (not relying on cell based expression from plasmids or viruses).

When referring to a sequence of the oligonucleotide, reference is made to the sequence or order of nucleobase moieties, or modifications thereof, of the covalently linked nucleotides or nucleosides. Generally, the oligonucleotide of the invention is man-made, and is chemically synthesized, and is typically purified or isolated. Although in some embodiments the oligonucleotide of the invention is a shRNA transcribed from a vector upon entry into the target cell. The oligonucleotide of the invention may comprise one or more modified nucleosides or nucleotides.

In some embodiments, the oligonucleotide of the invention comprises or consists of 10 to 70 nucleotides in length, such as from 12 to 60, such as from 13 to 50, such as from 14 to 40, such as from 15 to 30, such as from 12-25, such as from 16 to 22, such as from 16 to 20 contiguous nucleotides in length. Accordingly, the oligonucleotide of the present invention, in some embodiments, may have a length of 12-25 nucleotides. Alternatively, the oligonucleotide of the present invention, in some embodiments, may have a length of 15-22 nucleotides.

In some embodiments, the oligonucleotide or contiguous nucleotide sequence thereof comprises or consists of 24 or less nucleotides, such as 22, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 12 to 25 nucleotides, both 12 and 25 nucleotides are included.

In some embodiments, the contiguous nucleotide sequence comprises or consists of 12, 13, 14, 15, 16, 17, 18, 19, 20, 21 or 22 contiguous nucleotides in length

The olignucleotide(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s).

In one embodiment of the invention oligonucleotide is selected from a RNAi agent, such as a siRNA or shRNA. In another embodiment the oligonucleotide is a single stranded antisense oligonucleotide, such as a high affinity modified antisense oligonucleotide interacting with RNaseH.

In some embodiments the oligonucleotide of the invention may comprise one or more modified nucleosides or nucleotides, such as 2′ sugar modified nucleosides.

In some embodiments the oligonucleotide comprises phosphorothioate internucleoside linkages.

In some embodiments the oligonucleotide may be conjugated to non-nucleosidic moieties (conjugate moieties).

A library of oligonucleotides is to be understood as a collection of variant oligonucleotidess. The purpose of the library of oligonucleotides can vary. In some embodiments, the library of oligonucleotides is composed of oligonucleotides with overlapping nucleobase sequence targeting one or more mammalian RTEL1 target nucleic acids with the purpose of identifying the most potent sequence within the library of oligonucleotides. In some embodiments, the library of oligonucleotides is a library of oligonucleotide design variants (child nucleic acid molecules) of a parent or ancestral oligonucleotide, wherein the oligonucleotide design variants retaining the core nucleobase sequence of the parent nucleic acid molecule.

### Antisense Oligonucleotides

The term “antisense oligonucleotide” as used herein is defined as oligonucleotides capable of modulating expression of a target gene by hybridizing to a target nucleic acid, in particular to a contiguous sequence on a target nucleic acid. The antisense oligonucleotides herein are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide of the invention does not contain RNA nucleosides, since this will decrease nuclease resistance.

Advantageously, the oligonucleotide of the invention comprises one or more modified nucleosides or nucleotides, such as 2′ sugar modified nucleosides. Furthermore, it is advantageous that the nucleosides which are not modified are DNA nucleosides.

### RNAi Molecules

Herein, the term “RNA interference (RNAi) molecule” refers to short double-stranded RNA based oligonucleotide capable of inducing RNA-dependent gene silencing via the RNA-induced silencing complex (RISC) in a cell's cytoplasm, where they interact with the catalytic RISC component argonaute. The RNAi molecule modulates. e g., inhibits, the expression of the target nucleic acid in a cell. e.g. a cell within a subject. such as a mammalian subject. One type of RNAi molecule is a small interfering RNA (siRNA), which is a double-stranded RNA molecule composed of two complementary oligonucleotides, where the binding of one strand to complementary mRNA after transcription, leads to its degradation and loss of translation. A small hairpin RNA (shRNA) is a single stranded RNA-based oligonucleotide that forms a stem loop (hairpin) structure which is able to reduce mRNA via the DICER and RNA reducing silencing complex (RISC). RNAi molecules can be designed based on the sequence of the gene of interest (target nucleic acid). Corresponding RNAi can then be synthesized chemically or by in vitro transcription, or expressed from a vector or PCR product.

### SiRNA

The term siRNA refers to a small interfering ribonucleic acid RNAi molecule. It is a class of double-stranded RNA molecules, also known in the art as short interfering RNA or silencing RNA. siRNAs typically comprise a sense strand (also referred to as a passenger strand) and an antisense strand (also referred to as the guide strand), wherein each strand are of 17-30 nucleotides in length, typically 19-25 nucleosides in length, wherein the antisense strand is complementary, such as at least 95% complementary, such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and antisense strand 3′ ends may form a 3′ overhang of e.g. 1, 2 or 3 nucleosides to resemble the product produced by Dicer, which forms the RISC substrate in vivo. Effective extended forms of Dicer substrates have been described in U.S. Pat. Nos. 8,349,809 and 8,513,207, hereby incorporated by reference. In some embodiments, both the sense strand and antisense strand have a 2nt 3′ overhang. The duplex region may therefore be, for example 17-25 nucleotides in length, such as 21-23 nucleotide in length.

Once inside a cell the antisense strand is incorporated into the RISC complex which mediate target degradation or target inhibition of the target nucleic acid. siRNAs typically comprise modified nucleosides in addition to RNA nucleosides. In one embodiment the siRNA molecule may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. In particular 2′fluoro, 2′-O-methyl or 2′-O-methoxyethyl may be incorporated into siRNAs.

In some embodiments all of the nucleotides of an siRNA sense (passenger) strand may be modified with 2′ sugar modified nucleosides such as LNA (see WO2004/083430, WO2007/085485 for example). In some embodiments the passenger stand of the siRNA may be discontinuous (see WO2007/107162 for example). The incorporation of thermally destabilizing nucleotides occurring at a seed region of the antisense strand of siRNAs have been reported as useful in reducing off-target activity of siRNAs (see WO2018/098328 for example). Suitably the siRNA comprises a 5′ phosphate group or a 5′-phosphate mimic at the 5′ end of the antisense strand. In some embodiments the 5′ end of the antisense strand is a RNA nucleoside.

In one embodiment, the siRNA molecule further comprises at least one phosphorothioate or methylphosphonate internucleoside linkage. The phosphorothioaie or methylphosphonate internucleoside linkage may be at the 3′-terminus one or both strand (e.g., the antisense strand; or the sense strand); or the phosphorothioate or methylphosphonate internucleoside linkage may be at the 5′-terminus of one or both strands (e.g., the antisense strand; or the sense strand); or the phosphorothioate or methylphosphonate internucleoside linkage may be at the both the 5′- and 3′-terminus of one or both strands (e.g., the antisense strand; or the sense strand). In some embodiments the remaining internucleoside linkages are phosphodiester linkages. In some embodiments siRNA molecules comprise one or more phosphorothioate internucleoside linkages. In siRNA molecules phosphorothioate internucleoside linkages may reduce or the nuclease cleavage in RICS, it is therefore advantageous that not all internucleoside linkages in the antisense strand are modified.

The siRNA molecule may further comprise a ligand. In some embodiments, the ligand is conjugated to the 3′ end of the sense strand.

For biological distribution, siRNAs may be conjugated to a targeting ligand, and/or be formulated into lipid nanoparticles, for example.

Other aspects of the invention relate to pharmaceutical compositions comprising these dsRNA, such as siRNA molecules suitable for therapeutic use, and methods of inhibiting the expression of the target gene by administering the dsRNA molecules such as siRNAs of the invention, e.g., for the treatment of various disease conditions as disclosed herein.

### ShRNA

Short hairpin RNA or shRNA molecules are generally between 40 and 70 nucleotides in length, such as between 45 and 65 nucleotides in length, such as 50 and 60 nucleotides in length, and form a stem loop (hairpin) RNA structure, which interacts with the endonuclease known as Dicer which is believed to processes dsRNA into 19-23 base pair short interfering RNAs with characteristic two base 3′ overhangs which are then incorporated into an RNA-induced silencing complex (RISC). Upon binding to the appropriate target mRNA, one or more endonucleases within the RISC cleave the target to induce silencing. RNAi oligonucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA.

In some embodiments shRNA nucleic acid molecules comprise one or more phosphorothioate internucleoside linkages. In RNAi molecules phosphorothioate internucleoside linkages may reduce or the nuclease cleavage in RICS it is therefore advantageous that not al internucleoside linkages in the stem loop of the shRNA molecule are modified. Phosphorothioate internucleoside linkages can advantageously be place in the 3′ and/or 5′ end of the stem loop of the shRNA molecule, in particular in the of the part of the molecule that is not complementary to the target nucleic acid (e.g. the sense stand or passenger strand in an siRNA molecule). The region of the shRNA molecule that is complementary to the target nucleic acid may however also be modified in the first 2 to 3 internucleoside linkages in the part that is predicted to become the 3′ and/or 5′ terminal following cleavage by Dicer.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the contiguous nucleotide sequence is included in the guide strand of an siRNA molecule. In some embodiments the contiguous nucleotide sequence is the part of an shRNA molecule which is 100% complementary to the target nucleic acid. In some embodiments the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group for targeting) to the contiguous nucleotide sequence. The nucleotide linker region may or may not be complementary to the target nucleic acid. In some embodiments, the nucleobase sequence of the antisense oligonucleotide is the contiguous nucleotide sequence. In some embodiments, the contiguous nucleotide sequence is 100% complementary to the target nucleic acid.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages, such as a one or more phosphorothioate internucleoside linkages, or one or more phoshporodithioate internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region G of a gapmer oligonucleotide, as well as in regions of modified nucleosides, such as region F and F′.

In an embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

With the oligonucleotide of the invention it is advantageous to use phosphorothioate internucleoside linkages.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorthioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleoside, which according to EP 2 742 135 may for example be tolerated in an otherwise DNA phosphorothioate the gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides and DNA nucleosides. The antisense oligonucleotide of the invention is advantageously a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

The following is an example of an oligonucleotide motif (SEQ ID NO: 33) that is fully complementary to the target nucleic acid (SEQ ID NO: 11)

(SEQ ID NO: 11)
5′-CTTTGACCAGAGTATGTAAAATTCTC-3′
(SEQ ID NO: 33)
3′-AAACTGGTCTCATACATTTT-5′

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1 M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, the target nucleic acid is a nucleic acid which encodes mammalian RTEL1 and may for example be a gene, a RNA, a mRNA, and pre-mRNA, a mature mRNA or a cDNA sequence. The target may therefore be referred to as an RTEL1 target nucleic acid.

The oligonucleotide of the invention may for example target exon regions of a mammalian RTEL1 (in particular siRNA and shRNA target exon regions, but also antisense oligonucleotides), or may for example target intron region in the RTEL1 pre-mRNA (in particular antisense oligonucleotides target intron regions). The human RTEL1 gene encodes 15 transcripts of these 7 are protein coding and therefore potential nucleic acid targets. Table 1 lists predicted exon and intron regions of the 7 transcripts, as positioned on the human RTEL1 premRNA of SEQ ID NO: 1. It is understood that the oligonucleotides of the invention can target the mature mRNA sequence of one or more of the listed transcripts in table 1.

TABLE 1
Transcript-, exonic- and intronic regions in the human RTEL1 premRNA
(SEQ ID NO: 1) for the different protein coding RTEL1 mRNA transcripts

| | Transcript region | | Exonic regions | | | Intron regions | | |
|---|---|---|---|---|---|---|---|---|
| Transcript ID | start | end | Exon | start | end | intron | start | end |
| RTEL1-205 | 1 | 38444 | 1 | 1 | 657 | 1 | 657 | 1424 |
| ENST00000370018 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38444 | | | |
| RTEL1-203 | 485 | 38433 | 1 | 485 | 657 | 1 | 657 | 1424 |
| ENST00000360203 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37841 | 34 | 37841 | 37969 |
| | | | 35 | 37969 | 38433 | | | |
| RTEL1-212 | 482 | 38171 | 1 | 482 | 657 | 1 | 657 | 1424 |
| ENST00000508582 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4665 |
| | | | 5 | 4665 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38171 | | | |
| RTEL1-201 | 505 | 38434 | 1 | 505 | 650 | 1 | 650 | 3489 |
| ENST00000318100 | | | 2 | 3489 | 3687 | 2 | 3687 | 4041 |
| | | | 3 | 4041 | 4134 | 3 | 4134 | 4737 |
| | | | 4 | 4737 | 4818 | 4 | 4818 | 5020 |
| | | | 5 | 5020 | 5080 | 5 | 5080 | 8195 |
| | | | 6 | 8195 | 8270 | 6 | 8270 | 9660 |
| | | | 7 | 9660 | 9744 | 7 | 9744 | 14747 |
| | | | 8 | 14747 | 14812 | 8 | 14812 | 16131 |
| | | | 9 | 16131 | 16284 | 9 | 16284 | 20336 |
| | | | 10 | 20336 | 20374 | 10 | 20374 | 20459 |
| | | | 11 | 20459 | 20537 | 11 | 20537 | 22040 |
| | | | 12 | 22040 | 22137 | 12 | 22137 | 22855 |
| | | | 13 | 22855 | 22910 | 13 | 22910 | 27714 |
| | | | 14 | 27714 | 27788 | 14 | 27788 | 27982 |
| | | | 15 | 27982 | 28063 | 15 | 28063 | 29829 |
| | | | 16 | 29829 | 29961 | 16 | 29961 | 30128 |
| | | | 17 | 30128 | 30241 | 17 | 30241 | 30330 |
| | | | 18 | 30330 | 30370 | 18 | 30370 | 30492 |
| | | | 19 | 30492 | 30577 | 19 | 30577 | 30719 |
| | | | 20 | 30719 | 30796 | 20 | 30796 | 31246 |
| | | | 21 | 31246 | 31323 | 21 | 31323 | 31693 |
| | | | 22 | 31693 | 31839 | 22 | 31839 | 31941 |
| | | | 23 | 31941 | 32056 | 23 | 32056 | 32278 |
| | | | 24 | 32278 | 32401 | 24 | 32401 | 32485 |
| | | | 25 | 32485 | 32632 | 25 | 32632 | 32996 |
| | | | 26 | 32996 | 33138 | 26 | 33138 | 33933 |
| | | | 27 | 33933 | 34028 | 27 | 34028 | 34996 |
| | | | 28 | 34996 | 35194 | 28 | 35194 | 35334 |
| | | | 29 | 35334 | 35474 | 29 | 35474 | 36563 |
| | | | 30 | 36563 | 36679 | 30 | 36679 | 36932 |
| | | | 31 | 36932 | 37165 | 31 | 37165 | 37257 |
| | | | 32 | 37257 | 37412 | 32 | 37412 | 37519 |
| | | | 33 | 37519 | 37671 | 33 | 37671 | 37969 |
| | | | 34 | 37969 | 38434 | | | |
| RTEL1-202 | 551 | 16284 | 1 | 551 | 650 | 1 | 650 | 1424 |
| ENST00000356810 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4587 |
| | | | 5 | 4587 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | | | |
| RTEL1-206 | 30530 | 33067 | 1 | 30530 | 30577 | 1 | 30577 | 30719 |
| ENST00000425905 | | | 2 | 30719 | 30796 | 2 | 30796 | 31246 |
| | | | 3 | 31246 | 31323 | 3 | 31323 | 31941 |
| | | | 4 | 31941 | 32056 | 4 | 32056 | 32278 |
| | | | 5 | 32278 | 32401 | 5 | 32401 | 32485 |
| | | | 6 | 32485 | 32632 | 6 | 32632 | 32996 |
| | | | 7 | 32996 | 33067 | | | |
| RTEL1-214 | 811 | 3653 | 1 | 811 | 943 | 1 | 943 | 1424 |
| ENST00000646389 | | | 2 | 1424 | 1695 | 2 | 1695 | 3489 |
| | | | 3 | 3489 | 3653 | | | |

Suitably, the target nucleic acid encodes an RTEL1 protein, in particular mammalian RTEL1, such as human RTEL1 (See for example tables 2 and 3) which provides the pre-mRNA sequences for human and monkey, RTEL1.

In some embodiments, the target nucleic acid is selected from SEQ ID NO: 1 and/or 2 or naturally occurring variants thereof (e.g. sequences encoding a mammalian RTEL1 protein in table 1).

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the RTEL1 target nucleic acid in a cell which is expressing the RTEL1 target nucleic acid. The contiguous sequence of nucleobases of the oligonucleotide of the invention is typically complementary to the RTEL1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′or D″). The target nucleic acid may, in some embodiments, be a RNA or DNA, such as a messenger RNA, such as a mature mRNA (e.g. the exonic regions of the transcripts listed in table 1) or a pre-mRNA.

In some embodiments the target nucleic acid is a RNA or DNA which encodes mammalian RTEL1 protein, such as human RTEL1, e.g. the human RTEL1 mRNA sequence, such as that disclosed as SEQ ID NO 1. Further information on exemplary target nucleic acids is provided in tables 2 and 3.

TABLE 2
Genome and assembly information for RTEL1 across species.

| | | | Genomic coordinates | | | |
|---|---|---|---|---|---|---|
| Species | Chr. | Strand | Start | End | Assembly | ensembl gene_id |
| Human | 20 | fwd | 63657810 | 63696253 | GRCh38.p12 | ENSG00000258366 |
| Cynomolgus | 10 | fwd | 95853726 | 95890939 | Macaca_fascicularis_5.0 | ENSMFAG00000043680 |
| monkey | | | | | | |

Fwd = forward strand.
The genome coordinates provide the pre-mRNA sequence (genomic sequence).
The NCBI reference provides the mRNA sequence (cDNA sequence).

TABLE 3
Sequence details for RTEL1 across species.

| Species | RNA type | Length (nt) | SEQ ID NO |
|---|---|---|---|
| Human | premRNA | 38444 | 1 |
| Monkey | premRNA | 37214 | 2 |

Note SEQ ID NO 2 comprises regions of multiple NNNNs, where the sequencing has been unable to accurately refine the sequence, and a degenerate sequence is therefore included. For the avoidance of doubt the compounds of the invention are complementary to the actual target sequence and are not therefore degenerate compounds.

In some embodiments, the target nucleic acid is SEQ ID NO 1.

In some embodiments, the target nucleic acid is SEQ ID NO 2.

### Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides of the invention.

In some embodiments the target sequence is a sequence selected from the group consisting of a human RTEL1 mRNA exon, such as a RTEL1 human mRNA exon selected from the list in table 1 above.

In some embodiments the target sequence is a sequence selected from the group consisting of a human RTEL1 mRNA intron, such as a RTEL1 human mRNA intron selected from the list in table 1 above.

The oligonucleotide of the invention comprises a contiguous nucleotide sequence which is complementary to or hybridizes to the target nucleic acid, such as a target sequence described herein.

The target sequence to which the oligonucleotide is complementary or hybridizes to generally comprises a contiguous nucleobases sequence of at least 10 nucleotides. The contiguous nucleotide sequence is between 10 to 35 nucleotides, such as 12 to 30, such as 14 to 20, such as 16 to 20 contiguous nucleotides. In one embodiment of the invention the target sequence is selected from the group consisting of

SEQ ID NO: 3-21 as shown in table 4.

TABLE 4
Target sequences on human
RTEL1 prem RNA (SEQ ID NO: 1)

| | | Start | End |
|---|---|---|---|
| SEQ | | on SEQ | on SEQ |
| ID NO | Target Sequence | ID 1 | ID 1 |
| 3 | gaccactgtccttccatg | 8294 | 8311 |
| 4 | ttcagagattcaagttataataa | 8677 | 8722 |
| | agctcttcttatattgaggggga | | |
| 5 | aggaatagggttggtttt | 9377 | 9394 |
| 6 | ccttactacctgtcccg | 9667 | 9683 |
| 7 | acaattacttgttggatgcc | 9722 | 9741 |
| 8 | agcttctaacccaaccag | 10921 | 10938 |
| 9 | tataaacctaaatgtaaaagc | 11482 | 11502 |
| 10 | ttcaccaaaatttaaagctt | 11622 | 11641 |
| 11 | ctttgaccagagtatgtaaa | 11752 | 11777 |
| | attctc | | |
| 12 | aagacgtgttcaaagatt | 12868 | 12885 |
| 13 | ggacctactgttttttg | 13234 | 13250 |
| 14 | ggacctactgttttattcc | 13550 | 13568 |
| 15 | gtcccttctcttcctcctgtag | 14725 | 14746 |
| 16 | cgtgatctttgacgaagct | 14785 | 14803 |
| 17 | cgcaaacctttctgga | 14874 | 14889 |
| 18 | agcctgtgtgtggagtatgagca | 33025 | 33047 |
| 19 | cgtttccgtgttggtctggg | 34571 | 34590 |
| 20 | gactacaagggttccgatg | 35104 | 35122 |
| 21 | agtttgaggaggtctgtatc | 35370 | 35389 |

In some embodiments, the target sequence is selected from a region shown in Table 5A or 5B.

### Target Cell

The term a “target cell” as used herein refers to a cell which is expressing the target nucleic acid. For the therapeutic use of the present invention it is advantageous if the target cell is infected with HBV. In some embodiments the target cell may be in vivo or in vitro. In some embodiments the target cell is a mammalian cell such as a rodent cell, such as a mouse cell or a rat cell, or a woodchuck cell or a primate cell such as a monkey cell (e.g. a cynomolgus monkey cell) or a human cell.

In preferred embodiments the target cell expresses RTEL1 mRNA, such as the RTEL1 pre-mRNA or RTEL1 mature mRNA. The poly A tail of RTEL1 mRNA is typically disregarded for antisense oligonucleotide targeting.

Further, the target cell may be a hepatocyte. In one embodiment the target cell is HBV infected primary human hepatocytes, either derived from HBV infected individuals or from a HBV infected mouse with a humanized liver (PhoenixBio, PXB-mouse).

In accordance with the present invention, the target cell may be infected with HBV. Further, the target cell may comprise HBV cccDNA. Thus, the target cell preferably comprises RTEL1 mRNA, such as the RTEL1 pre-mRNA or RTEL1 mature mRNA, and HBV cccDNA.

### Naturally Occurring Variant

The term “naturally occurring variant” refers to variants of RTEL1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian RTEL1 target nucleic acid, such as a target nucleic acid of SEQ ID NO 1 and/or 2. In some embodiments the naturally occurring variants have at least 99% homology to the human RTEL1 target nucleic acid of SEQ ID NO: 1. In some embodiments the naturally occurring variants are known polymorphisms.

### Modulation of Expression

The term “modulation of expression” as used herein is to be understood as an overall term for an oligonucleotide's ability to alter the amount of RTEL1 when compared to the amount of RTEL1 before administration of the oligonucleotide. Alternatively, modulation of expression may be determined by reference to a control experiment. It is generally understood that the control is an individual or target cell treated with a saline composition or an individual or target cell treated with a non-targeting oligonucleotide (mock).

One type of modulation is the ability of an oligonucleotide to inhibit, down-regulate, reduce, suppress, remove, stop, block, prevent, lessen, lower, avoid or terminate expression of RTEL1, e.g. by degradation of mRNA or blockage of transcription. Another type of modulation is an oligonucleotide's ability to restore, increase or enhance expression of RTEL1, e.g. by repair of splice sites or prevention of splicing or removal or blockage of inhibitory mechanisms such as microRNA repression.

### Sugar Modifications

The oligonucleotide of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between+3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

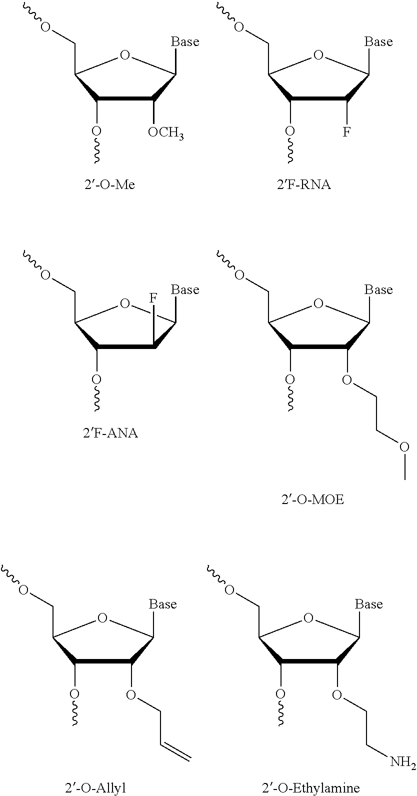

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667.

Particular examples of LNA nucleosides of the invention are presented in Scheme 1 (wherein B is as defined above).

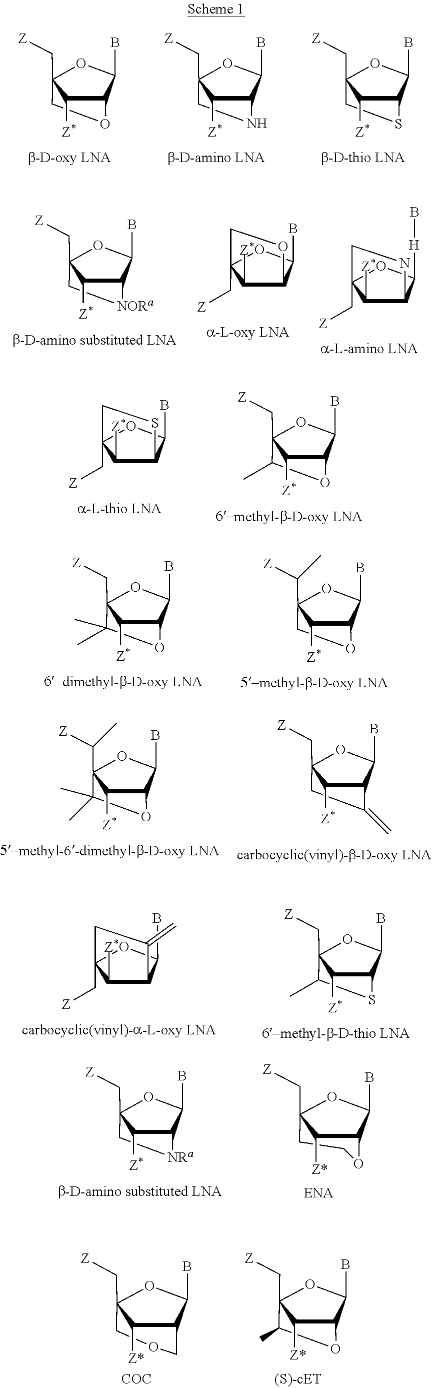

Particular LNA nucleosides are beta-D-oxy-LNA, 6′-methyl-beta-D-oxy LNA such as (S)-6′-methyl-beta-D-oxy-LNA (ScET) and ENA.

### Pharmaceutically Acceptable Salts

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO 01/23613 (hereby incorporated by reference). For use in determining RHase H activity, recombinant human RNase H1 is available from Creative Biomart® (Recombinant Human RNASEH1 fused with His tag expressed in E. coli).

### Gapmer

The antisense oligonucleotide of the invention, or contiguous nucleotide sequence thereof, may be a gapmer, also termed gapmer oligonucleotide or gapmer designs. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to17, such as 16 to18 nucleosides. By way of example, the gapmer oligonucleotide of the present invention can be represented by the following formulae:

F₁₋₈-G₅₋₁₈-F′₁₋₈, such as
F₁₋₈-G₅₋₁₆-F′₁₋₈, such as
F₁₋₈-G₇₋₁₆-F′₂₋₈
with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 18, such as 6 and 16, nucleosides which are capable of recruiting RNaseH. In some embodiments the G region consists of DNA nucleosides.

Regions F, G and F′ are further defined below and can be incorporated into the F-G-F′ formula.

### Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-18 contiguous DNA nucleosides, 5-17 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15, 16, 17 or 18 contiguous DNA nucleosides. Cytosine (C) DNA in the gap region may in some instances be methylated, such residues are either annotated as 5′-methyl-cytosine (^{me}C or with an e instead of a c). Methylation of cytosine DNA in the gap is advantageous if cg dinucleotides are present in the gap to reduce potential toxicity, the modification does not have significant impact on efficacy of the oligonucleotides. 5′ substituted DNA nucleosides, such as 5′ methyl DNA nucleoside have been reported for use in DNA gap regions (EP 2 742 136).

In some embodiments the gap region G may consist of 6, 7, 8, 9, 10, 11, 12, 13, 14, 15, 16, 17 or 18 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

### Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region For F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

### MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

### Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′or D″ constitute a separate part of the oligonucleotide.

Region D′or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈
D′-F-G-F′, in particular D′₁₋₃-F₁₋₈-G₅₋₁₆-F′₂₋₈
F-G-F′-D″, in particular F₁₋₈-G₅₋₁₆-F′₂₋₈-D″₁₋₃
D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

### Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs.

WO 93/07883 and WO2013/033230 provides suitable conjugate moieties, which are hereby incorporated by reference. Further suitable conjugate moieties are those capable of binding to the asialoglycoprotein receptor (ASGPR). In particular, tri-valent N-acetylgalactosamine conjugate moieties are suitable for binding to the ASGPR, see for example WO 2014/076196, WO 2014/207232 and WO 2014/179620 (hereby incorporated by reference). Such conjugates serve to enhance uptake of the oligonucleotide to the liver while reducing its presence in the kidney, thereby increasing the liver/kidney ratio of a conjugated oligonucleotide compared to the unconjugated version of the same oligonucleotide.

Oligonucleotide conjugates and their synthesis has also been reported in comprehensive reviews by Manoharan in Antisense Drug Technology, Principles, Strategies, and Applications, S.T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Prophylactic can be understood as preventing an HBV infection from turning into a chronic HBV infection or the prevention of severe liver diseases such as liver cirrhosis and hepatocellular carcinoma caused by a chronic HBV infection.

### Prevention

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof. Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is human.

### DETAILED DESCRIPTION OF THE INVENTION

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that RTEL1 is associated with cccDNA stability. This knowledge allows for the opportunity to destabilize cccDNA in HBV infected subjects which in turn opens the opportunity for a complete cure of chronically infected HBV patients.

One aspect of the present invention is a RTEL1 inhibitor for use in the treatment and/or prevention of Hepatitis B virus (HBV) infection, in particular a chronic HBV infection.

The RTEL1 inhibitor can for example be a small molecule that specifically binds to RTEL1 protein, wherein said inhibitor prevents or reduces binding of RTEL1 protein to cccDNA.

An embodiment of the invention is a RTEL1 inhibitor which is capable of reducing cccDNA and/or pgRNA in an infected cell, such as an HBV infected cell.

In a further embodiment, the RTEL1 inhibitor is capable of reducing HBsAg and/or HBeAg in vivo in an HBV infected individual.

### The Oligonucleotides of the Invention

Therapeutic oligonucleotides are potentially excellent RTEL1 inhibitors since they can target the RTEL1 transcript and promote its degradation either via the RNA interference pathway or via RNaseH cleavage. Alternatively, oligonucleotides such as aptamers can also act as inhibitors of RTEL1 protein interactions.

One aspect of the present invention is a RTEL1 targeting oligonucleotide for use in treatment and/or prevention of Hepatitis B virus (HBV) infection. Such an oligonucleotide can be selected from the group consisting of single stranded antisense oligonucleotide; siRNA molecule; or shRNA molecule.

The present section describes novel oligonucleotides suitable for use in treatment and/or prevention of Hepatitis B virus (HBV) infection.

The oligonucleotides of the present invention are capable of inhibiting expression of RTEL1 in vitro and in vivo. The inhibition is achieved by hybridizing an oligonucleotide to a target nucleic acid encoding RTEL1 or which is involved in the regulation of RTEL1. The target nucleic acid may be a mammalian RTEL1 sequence, such as the sequence of SEQ ID NO: 1 and/or 2

In some embodiments the oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. In some embodiments, the oligonucleotide of the invention may be capable of inhibiting expression levels of RTEL1 mRNA by at least 60% or 70% in vitro using 10 μM in PXB-PHH cells. In some embodiments, the oligonucleotide of the invention may be capable of inhibiting expression levels of RTEL1 protein by at least 50% in vitro using 10 μM PXB-PHH cells, this range of target reduction is advantageous in terms of selecting nucleic acid molecules with good correlation to the cccDNA reduction. Suitably, the examples provide assays which may be used to measure RTEL1 RNA or protein inhibition (e.g. example 1). The target inhibition is triggered by the hybridization between a contiguous nucleotide sequence of the oligonucleotide and the target nucleic acid. In some embodiments, the oligonucleotide of the invention comprises mismatches between the oligonucleotide and the target nucleic acid. Despite mismatches hybridization to the target nucleic acid may still be sufficient to show a desired inhibition of RTEL1 expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

An aspect of the present invention relates to an oligonucleotides of 12 to 60 nucleotides in length, which comprises a contiguous nucleotide sequence of at least 10 nucleotides in length, such as at least 12 to 30 nucleotides in length, which is at least 95% complementary, such as fully complementary, to a mammalian RTEL1 target nucleic acid, in particular a human RTEL1 nucleic acid. These oligonucleotides are capable of inhibiting the expression of RTEL1.

An aspect of the invention relates to an oligonucleotide according to the invention which is an antisense oligonucleotide of 12 to 30 nucleotides in length, comprising a contiguous nucleotide sequence of at least 10 nucleotides, such as 10 to 30 nucleotides in length which is at least 90% complementary, such as fully complementary, to a mammalian RTEL1.

A further aspect of the present invention relates to an oligonucleotide according to the invention comprising a contiguous nucleotide sequence of 12 to 20, such as 15 to 22, nucleotides in length with at least 90% complementarity, such as fully complementary, to the target nucleic acid of SEQ ID NO: 1.

In some embodiments, the oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the oligonucleotide of the invention, or contiguous nucleotide sequence thereof is fully complementary (100% complementary) to a region of the target nucleic acid, or in some embodiments may comprise one or two mismatches between the oligonucleotide and the target nucleic acid.

In some embodiments the antisense oligonucleotide sequence is 100% complementary to a corresponding target nucleic acid of SEQ ID NO: 1.

In some embodiments the oligonucleotide or the contiguous nucleotide sequence of the invention is at least 95% complementarity, such as fully (or 100%) complementary, to the target nucleic acid of SEQ ID NO: 1 and SEQ ID NO: 2.

In some embodiments, the oligonucleotide comprises a contiguous nucleotide sequence of 15 to 22 nucleotides in length with at least 90% complementary, such as 100% complementarity, to a corresponding target sequence present in SEQ ID NO: 1, wherein the target sequence is selected from the group consisting of SEQ ID NO: 3 to 21 (table 4) or region 1A to 959A in Table 5A.

TABLE 5A
Regions of SEQ ID NO 1 which may be
targeted using an oligonucleotide of the invention

| | Position in SEQ ID NO 1 | | |
|---|---|---|---|
| Reg. A | from | to | Length |
| 1A | 1594 | 1623 | 30 |
| 2A | 1636 | 1685 | 50 |
| 3A | 1687 | 1708 | 22 |
| 4A | 1773 | 1794 | 22 |
| 5A | 1810 | 1824 | 15 |
| 6A | 1824 | 1870 | 47 |
| 7A | 2890 | 2907 | 18 |
| 8A | 2931 | 2952 | 22 |
| 9A | 2984 | 2999 | 16 |
| 10A | 3002 | 3021 | 20 |
| 11A | 3026 | 3081 | 56 |
| 12A | 3083 | 3100 | 18 |
| 13A | 3175 | 3202 | 28 |
| 14A | 3205 | 3228 | 24 |
| 15A | 3253 | 3270 | 18 |
| 16A | 3289 | 3320 | 32 |
| 17A | 3353 | 3368 | 16 |
| 18A | 3374 | 3388 | 15 |
| 19A | 3443 | 3472 | 30 |
| 20A | 3525 | 3547 | 23 |
| 21A | 3549 | 3581 | 33 |
| 22A | 3584 | 3612 | 29 |
| 23A | 3648 | 3675 | 28 |
| 24A | 3681 | 3708 | 28 |
| 25A | 3716 | 3731 | 16 |
| 26A | 3733 | 3755 | 23 |
| 27A | 3757 | 3775 | 19 |
| 28A | 3797 | 3811 | 15 |
| 29A | 3823 | 3844 | 22 |
| 30A | 3846 | 3881 | 36 |
| 31A | 3922 | 3955 | 34 |
| 32A | 3957 | 3975 | 19 |
| 33A | 4016 | 4036 | 21 |
| 34A | 4038 | 4053 | 16 |
| 35A | 4067 | 4098 | 32 |
| 36A | 4100 | 4150 | 51 |
| 37A | 4271 | 4290 | 20 |
| 38A | 4312 | 4330 | 19 |
| 39A | 4345 | 4362 | 18 |
| 40A | 4364 | 4379 | 16 |
| 41A | 4420 | 4448 | 29 |
| 42A | 4480 | 4512 | 33 |
| 43A | 4527 | 4552 | 26 |
| 44A | 4651 | 4674 | 24 |
| 45A | 4733 | 4784 | 52 |
| 46A | 4786 | 4811 | 26 |
| 47A | 4830 | 4848 | 19 |
| 48A | 4857 | 4874 | 18 |
| 49A | 4915 | 4937 | 23 |
| 50A | 4944 | 4966 | 23 |
| 51A | 4969 | 4983 | 15 |
| 52A | 4995 | 5015 | 21 |
| 53A | 5017 | 5033 | 17 |
| 54A | 5035 | 5075 | 41 |
| 55A | 5109 | 5136 | 28 |
| 56A | 5155 | 5173 | 19 |
| 57A | 5175 | 5191 | 17 |
| 58A | 5209 | 5225 | 17 |
| 59A | 5241 | 5261 | 21 |
| 60A | 5263 | 5287 | 25 |
| 61A | 5299 | 5346 | 48 |
| 62A | 5394 | 5409 | 16 |
| 63A | 5428 | 5447 | 20 |
| 64A | 5472 | 5514 | 43 |
| 65A | 5579 | 5606 | 28 |
| 66A | 5617 | 5632 | 16 |
| 67A | 5690 | 5711 | 22 |
| 68A | 5713 | 5754 | 42 |
| 69A | 5727 | 5741 | 15 |
| 70A | 5756 | 5770 | 15 |
| 71A | 5812 | 5854 | 43 |
| 72A | 5871 | 5886 | 16 |
| 73A | 5896 | 5932 | 37 |
| 74A | 5992 | 6009 | 18 |
| 75A | 6011 | 6038 | 28 |
| 76A | 6057 | 6072 | 16 |
| 77A | 6101 | 6122 | 22 |
| 78A | 6127 | 6165 | 39 |
| 79A | 6187 | 6203 | 17 |
| 80A | 6210 | 6227 | 18 |
| 81A | 6243 | 6261 | 19 |
| 82A | 6271 | 6299 | 29 |
| 83A | 6378 | 6393 | 16 |
| 84A | 6468 | 6496 | 29 |
| 85A | 6498 | 6526 | 29 |
| 86A | 6558 | 6574 | 17 |
| 87A | 6720 | 6737 | 18 |
| 88A | 6735 | 6749 | 15 |
| 89A | 6785 | 6825 | 41 |
| 90A | 6879 | 6894 | 16 |
| 91A | 6921 | 6959 | 39 |
| 92A | 6995 | 7060 | 66 |
| 93A | 7062 | 7084 | 23 |
| 94A | 7114 | 7146 | 33 |
| 95A | 7155 | 7186 | 32 |
| 96A | 7188 | 7203 | 16 |
| 97A | 7230 | 7267 | 38 |
| 98A | 7281 | 7299 | 19 |
| 99A | 7291 | 7316 | 26 |
| 100A | 7344 | 7369 | 26 |
| 101A | 7375 | 7391 | 17 |
| 102A | 7400 | 7414 | 15 |
| 103A | 7427 | 7441 | 15 |
| 104A | 7437 | 7453 | 17 |
| 105A | 7449 | 7463 | 15 |
| 106A | 7467 | 7481 | 15 |
| 107A | 7500 | 7518 | 19 |
| 108A | 7532 | 7546 | 15 |
| 109A | 7573 | 7587 | 15 |
| 110A | 7607 | 7621 | 15 |
| 111A | 7659 | 7685 | 27 |
| 112A | 7732 | 7767 | 36 |
| 113A | 7779 | 7793 | 15 |
| 114A | 7844 | 7882 | 39 |
| 115A | 7888 | 7910 | 23 |
| 116A | 7966 | 7980 | 15 |
| 117A | 8033 | 8047 | 15 |
| 118A | 8049 | 8063 | 15 |
| 119A | 8160 | 8178 | 19 |
| 120A | 8180 | 8195 | 16 |
| 121A | 8216 | 8237 | 22 |
| 122A | 8239 | 8341 | 103 |
| 123A | 8357 | 8373 | 17 |
| 124A | 8415 | 8430 | 16 |
| 125A | 8449 | 8465 | 17 |
| 126A | 8541 | 8560 | 20 |
| 127A | 8574 | 8596 | 23 |
| 128A | 8677 | 8703 | 27 |
| 129A | 8705 | 8722 | 18 |
| 130A | 8748 | 8763 | 16 |
| 131A | 8792 | 8807 | 16 |
| 132A | 8796 | 8811 | 16 |
| 133A | 8799 | 8818 | 20 |
| 134A | 8807 | 8821 | 15 |
| 135A | 8814 | 8828 | 15 |
| 136A | 8837 | 8853 | 17 |
| 137A | 8837 | 8851 | 15 |
| 138A | 8841 | 8858 | 18 |
| 139A | 8884 | 8911 | 28 |
| 140A | 8918 | 8937 | 20 |
| 141A | 8918 | 8933 | 16 |
| 142A | 8969 | 9005 | 37 |
| 143A | 8969 | 8999 | 31 |
| 144A | 8969 | 9000 | 32 |
| 145A | 8971 | 8989 | 19 |
| 146A | 8974 | 9000 | 27 |
| 147A | 8982 | 8999 | 18 |
| 148A | 9024 | 9042 | 19 |
| 149A | 9026 | 9042 | 17 |
| 150A | 9026 | 9040 | 15 |
| 151A | 9026 | 9041 | 16 |
| 152A | 9027 | 9043 | 17 |
| 153A | 9027 | 9041 | 15 |
| 154A | 9027 | 9042 | 16 |
| 155A | 9028 | 9044 | 17 |
| 156A | 9028 | 9042 | 15 |
| 157A | 9028 | 9043 | 16 |
| 158A | 9029 | 9045 | 17 |
| 159A | 9029 | 9043 | 15 |
| 160A | 9029 | 9044 | 16 |
| 161A | 9030 | 9046 | 17 |
| 162A | 9030 | 9044 | 15 |
| 163A | 9030 | 9045 | 16 |
| 164A | 9031 | 9047 | 17 |
| 165A | 9031 | 9045 | 15 |
| 166A | 9031 | 9046 | 16 |
| 167A | 9032 | 9048 | 17 |
| 168A | 9032 | 9046 | 15 |
| 169A | 9032 | 9047 | 16 |
| 170A | 9033 | 9047 | 15 |
| 171A | 9033 | 9048 | 16 |
| 172A | 9034 | 9048 | 15 |
| 173A | 9038 | 9052 | 15 |
| 174A | 9046 | 9064 | 19 |
| 175A | 9069 | 9090 | 22 |
| 176A | 9074 | 9089 | 16 |
| 177A | 9078 | 9093 | 16 |
| 178A | 9095 | 9110 | 16 |
| 179A | 9101 | 9124 | 24 |
| 180A | 9126 | 9161 | 36 |
| 181A | 9135 | 9155 | 21 |
| 182A | 9147 | 9162 | 16 |
| 183A | 9163 | 9186 | 24 |
| 184A | 9203 | 9222 | 20 |
| 185A | 9210 | 9253 | 44 |
| 186A | 9210 | 9230 | 21 |
| 187A | 9223 | 9254 | 32 |
| 188A | 9241 | 9256 | 16 |
| 189A | 9258 | 9272 | 15 |
| 190A | 9266 | 9303 | 38 |
| 191A | 9291 | 9308 | 18 |
| 192A | 9311 | 9329 | 19 |
| 193A | 9370 | 9394 | 25 |
| 194A | 9406 | 9420 | 15 |
| 195A | 9569 | 9591 | 23 |
| 196A | 9653 | 9708 | 56 |
| 197A | 9712 | 9758 | 47 |
| 198A | 9771 | 9788 | 18 |
| 199A | 9812 | 9829 | 18 |
| 200A | 9844 | 9862 | 19 |
| 201A | 9872 | 9917 | 46 |
| 202A | 9958 | 9983 | 26 |
| 203A | 9985 | 10002 | 18 |
| 204A | 10017 | 10054 | 38 |
| 205A | 10113 | 10132 | 20 |
| 206A | 10113 | 10130 | 18 |
| 207A | 10120 | 10137 | 18 |
| 208A | 10183 | 10204 | 22 |
| 209A | 10185 | 10204 | 20 |
| 210A | 10185 | 10202 | 18 |
| 211A | 10192 | 10209 | 18 |
| 212A | 10192 | 10210 | 19 |
| 213A | 10231 | 10251 | 21 |
| 214A | 10236 | 10251 | 16 |
| 215A | 10320 | 10337 | 18 |
| 216A | 10338 | 10353 | 16 |
| 217A | 10397 | 10415 | 19 |
| 218A | 10563 | 10584 | 22 |
| 219A | 10591 | 10607 | 17 |
| 220A | 10703 | 10723 | 21 |
| 221A | 10766 | 10784 | 19 |
| 222A | 10805 | 10822 | 18 |
| 223A | 10844 | 10870 | 27 |
| 224A | 10873 | 10893 | 21 |
| 225A | 10895 | 10913 | 19 |
| 226A | 10915 | 10942 | 28 |
| 227A | 10961 | 10975 | 15 |
| 228A | 10983 | 10999 | 17 |
| 229A | 11001 | 11015 | 15 |
| 230A | 11021 | 11035 | 15 |
| 231A | 11033 | 11059 | 27 |
| 232A | 11061 | 11082 | 22 |
| 233A | 11084 | 11104 | 21 |
| 234A | 11124 | 11154 | 31 |
| 235A | 11156 | 11170 | 15 |
| 236A | 11175 | 11192 | 18 |
| 237A | 11227 | 11260 | 34 |
| 238A | 11239 | 11254 | 16 |
| 239A | 11274 | 11302 | 29 |
| 240A | 11290 | 11305 | 16 |
| 241A | 11299 | 11317 | 19 |
| 242A | 11305 | 11329 | 25 |
| 243A | 11344 | 11361 | 18 |
| 244A | 11372 | 11400 | 29 |
| 245A | 11402 | 11416 | 15 |
| 246A | 11418 | 11445 | 28 |
| 247A | 11457 | 11471 | 15 |
| 248A | 11482 | 11511 | 30 |
| 249A | 11550 | 11566 | 17 |
| 250A | 11622 | 11645 | 24 |
| 251A | 11722 | 11737 | 16 |
| 252A | 11745 | 11777 | 33 |
| 253A | 11824 | 11844 | 21 |
| 254A | 11824 | 11840 | 17 |
| 255A | 12622 | 12638 | 17 |
| 256A | 12673 | 12691 | 19 |
| 257A | 12693 | 12724 | 32 |
| 258A | 12747 | 12763 | 17 |
| 259A | 12783 | 12806 | 24 |
| 260A | 12818 | 12837 | 20 |
| 261A | 12856 | 12885 | 30 |
| 262A | 12890 | 12912 | 23 |
| 263A | 12914 | 12945 | 32 |
| 264A | 12984 | 13016 | 33 |
| 265A | 13001 | 13016 | 16 |
| 266A | 13004 | 13022 | 19 |
| 267A | 13004 | 13021 | 18 |
| 268A | 13014 | 13034 | 21 |
| 269A | 13166 | 13191 | 26 |
| 270A | 13228 | 13251 | 24 |
| 271A | 13283 | 13319 | 37 |
| 272A | 13295 | 13310 | 16 |
| 273A | 13317 | 13332 | 16 |
| 274A | 13354 | 13381 | 28 |
| 275A | 13383 | 13430 | 48 |
| 276A | 13446 | 13468 | 23 |
| 277A | 13449 | 13468 | 20 |
| 278A | 13471 | 13487 | 17 |
| 279A | 13500 | 13518 | 19 |
| 280A | 13547 | 13568 | 22 |
| 281A | 13631 | 13650 | 20 |
| 282A | 13663 | 13679 | 17 |
| 283A | 13680 | 13694 | 15 |
| 284A | 13744 | 13764 | 21 |
| 285A | 13766 | 13803 | 38 |
| 286A | 13768 | 13803 | 36 |
| 287A | 13777 | 13797 | 21 |
| 288A | 13789 | 13804 | 16 |
| 289A | 13804 | 13827 | 24 |
| 290A | 13823 | 13844 | 22 |
| 291A | 13840 | 13854 | 15 |
| 292A | 13840 | 13855 | 16 |
| 293A | 13841 | 13855 | 15 |
| 294A | 13851 | 13874 | 24 |
| 295A | 13851 | 13873 | 23 |
| 296A | 13853 | 13871 | 19 |
| 297A | 13855 | 13874 | 20 |
| 298A | 13862 | 13882 | 21 |
| 299A | 13890 | 13905 | 16 |
| 300A | 13897 | 13927 | 31 |
| 301A | 13926 | 13940 | 15 |
| 302A | 13957 | 13971 | 15 |
| 303A | 13966 | 13980 | 15 |
| 304A | 13995 | 14025 | 31 |
| 305A | 14027 | 14048 | 22 |
| 306A | 14048 | 14067 | 20 |
| 307A | 14084 | 14098 | 15 |
| 308A | 14118 | 14133 | 16 |
| 309A | 14154 | 14171 | 18 |
| 310A | 14173 | 14210 | 38 |
| 311A | 14198 | 14218 | 21 |
| 312A | 14200 | 14218 | 19 |
| 313A | 14237 | 14265 | 29 |
| 314A | 14242 | 14265 | 24 |
| 315A | 14242 | 14264 | 23 |
| 316A | 14244 | 14262 | 19 |
| 317A | 14246 | 14265 | 20 |
| 318A | 14253 | 14271 | 19 |
| 319A | 14273 | 14293 | 21 |
| 320A | 14290 | 14304 | 15 |
| 321A | 14295 | 14320 | 26 |
| 322A | 14308 | 14352 | 45 |
| 323A | 14323 | 14352 | 30 |
| 324A | 14326 | 14352 | 27 |
| 325A | 14334 | 14351 | 18 |
| 326A | 14340 | 14364 | 25 |
| 327A | 14340 | 14359 | 20 |
| 328A | 14348 | 14362 | 15 |
| 329A | 14374 | 14406 | 33 |
| 330A | 14416 | 14446 | 31 |
| 331A | 14462 | 14489 | 28 |
| 332A | 14505 | 14521 | 17 |
| 333A | 14523 | 14541 | 19 |
| 334A | 14577 | 14598 | 22 |
| 335A | 14725 | 14762 | 38 |
| 336A | 14764 | 14781 | 18 |
| 337A | 14783 | 14808 | 26 |
| 338A | 14874 | 14905 | 32 |
| 339A | 14974 | 15030 | 57 |
| 340A | 15032 | 15059 | 28 |
| 341A | 15084 | 15098 | 15 |
| 342A | 15087 | 15106 | 20 |
| 343A | 15108 | 15126 | 19 |
| 344A | 15147 | 15180 | 34 |
| 345A | 15183 | 15202 | 20 |
| 346A | 15230 | 15247 | 18 |
| 347A | 15255 | 15270 | 16 |
| 348A | 15272 | 15298 | 27 |
| 349A | 15288 | 15312 | 25 |
| 350A | 15319 | 15349 | 31 |
| 351A | 15359 | 15373 | 15 |
| 352A | 15370 | 15385 | 16 |
| 353A | 15382 | 15400 | 19 |
| 354A | 15388 | 15408 | 21 |
| 355A | 15410 | 15435 | 26 |
| 356A | 15435 | 15452 | 18 |
| 357A | 15456 | 15498 | 43 |
| 358A | 15459 | 15474 | 16 |
| 359A | 15479 | 15498 | 20 |
| 360A | 15486 | 15502 | 17 |
| 361A | 15528 | 15543 | 16 |
| 362A | 15543 | 15561 | 19 |
| 363A | 15572 | 15591 | 20 |
| 364A | 15623 | 15642 | 20 |
| 365A | 15646 | 15660 | 15 |
| 366A | 15662 | 15690 | 29 |
| 367A | 15702 | 15740 | 39 |
| 368A | 15740 | 15754 | 15 |
| 369A | 15743 | 15773 | 31 |
| 370A | 15746 | 15761 | 16 |
| 371A | 15764 | 15789 | 26 |
| 372A | 15777 | 15803 | 27 |
| 373A | 15791 | 15816 | 26 |
| 374A | 15832 | 15848 | 17 |
| 375A | 15855 | 15873 | 19 |
| 376A | 15870 | 15890 | 21 |
| 377A | 15878 | 15908 | 31 |
| 378A | 15880 | 15898 | 19 |
| 379A | 15891 | 15908 | 18 |
| 380A | 15896 | 15916 | 21 |
| 381A | 15911 | 15929 | 19 |
| 382A | 15911 | 15930 | 20 |
| 383A | 15947 | 15963 | 17 |
| 384A | 16023 | 16056 | 34 |
| 385A | 16068 | 16091 | 24 |
| 386A | 16083 | 16097 | 15 |
| 387A | 16129 | 16150 | 22 |
| 388A | 16170 | 16229 | 60 |
| 389A | 16245 | 16265 | 21 |
| 390A | 16269 | 16300 | 32 |
| 391A | 16308 | 16334 | 27 |
| 392A | 16336 | 16356 | 21 |
| 393A | 16336 | 16358 | 23 |
| 394A | 16360 | 16391 | 32 |
| 395A | 16360 | 16397 | 38 |
| 396A | 16425 | 16465 | 41 |
| 397A | 16472 | 16493 | 22 |
| 398A | 16498 | 16515 | 18 |
| 399A | 16545 | 16562 | 18 |
| 400A | 16564 | 16586 | 23 |
| 401A | 16588 | 16613 | 26 |
| 402A | 16615 | 16639 | 25 |
| 403A | 16651 | 16667 | 17 |
| 404A | 16669 | 16695 | 27 |
| 405A | 16696 | 16716 | 21 |
| 406A | 16704 | 16718 | 15 |
| 407A | 16732 | 16760 | 29 |
| 408A | 16737 | 16760 | 24 |
| 409A | 16849 | 16865 | 17 |
| 410A | 16853 | 16868 | 16 |
| 411A | 16853 | 16867 | 15 |
| 412A | 16882 | 16897 | 16 |
| 413A | 16885 | 16902 | 18 |
| 414A | 16914 | 16938 | 25 |
| 415A | 16942 | 16956 | 15 |
| 416A | 16990 | 17004 | 15 |
| 417A | 17016 | 17042 | 27 |
| 418A | 17097 | 17115 | 19 |
| 419A | 17105 | 17119 | 15 |
| 420A | 17105 | 17126 | 22 |
| 421A | 17114 | 17128 | 15 |
| 422A | 17133 | 17158 | 26 |
| 423A | 17160 | 17174 | 15 |
| 424A | 17162 | 17178 | 17 |
| 425A | 17166 | 17183 | 18 |
| 426A | 17178 | 17199 | 22 |
| 427A | 17187 | 17201 | 15 |
| 428A | 17203 | 17223 | 21 |
| 429A | 17213 | 17230 | 18 |
| 430A | 17213 | 17235 | 23 |
| 431A | 17237 | 17259 | 23 |
| 432A | 17249 | 17279 | 31 |
| 433A | 17267 | 17285 | 19 |
| 434A | 17273 | 17288 | 16 |
| 435A | 17297 | 17315 | 19 |
| 436A | 17300 | 17315 | 16 |
| 437A | 17302 | 17317 | 16 |
| 438A | 17303 | 17324 | 22 |
| 439A | 17312 | 17330 | 19 |
| 440A | 17346 | 17375 | 30 |
| 441A | 17349 | 17375 | 27 |
| 442A | 17357 | 17374 | 18 |
| 443A | 17363 | 17382 | 20 |
| 444A | 17371 | 17385 | 15 |
| 445A | 17420 | 17447 | 28 |
| 446A | 17524 | 17551 | 28 |
| 447A | 17562 | 17580 | 19 |
| 448A | 17622 | 17636 | 15 |
| 449A | 17702 | 17734 | 33 |
| 450A | 17730 | 17745 | 16 |
| 451A | 17733 | 17755 | 23 |
| 452A | 17743 | 17758 | 16 |
| 453A | 17810 | 17824 | 15 |
| 454A | 17900 | 17940 | 41 |
| 455A | 17942 | 17968 | 27 |
| 456A | 17988 | 18002 | 15 |
| 457A | 18007 | 18024 | 18 |
| 458A | 18026 | 18042 | 17 |
| 459A | 18044 | 18059 | 16 |
| 460A | 18126 | 18159 | 34 |
| 461A | 18179 | 18205 | 27 |
| 462A | 18237 | 18253 | 17 |
| 463A | 18272 | 18290 | 19 |
| 464A | 18299 | 18314 | 16 |
| 465A | 18328 | 18344 | 17 |
| 466A | 18329 | 18344 | 16 |
| 467A | 18347 | 18361 | 15 |
| 468A | 18380 | 18402 | 23 |
| 469A | 18385 | 18399 | 15 |
| 470A | 18406 | 18421 | 16 |
| 471A | 18446 | 18473 | 28 |
| 472A | 18527 | 18543 | 17 |
| 473A | 18554 | 18569 | 16 |
| 474A | 18631 | 18645 | 15 |
| 475A | 18673 | 18693 | 21 |
| 476A | 18746 | 18765 | 20 |
| 477A | 18797 | 18824 | 28 |
| 478A | 18842 | 18860 | 19 |
| 479A | 18872 | 18892 | 21 |
| 480A | 18901 | 18915 | 15 |
| 481A | 18901 | 18940 | 40 |
| 482A | 18942 | 18976 | 35 |
| 483A | 18951 | 18976 | 26 |
| 484A | 18971 | 18994 | 24 |
| 485A | 18998 | 19016 | 19 |
| 486A | 19020 | 19039 | 20 |
| 487A | 19027 | 19043 | 17 |
| 488A | 19027 | 19050 | 24 |
| 489A | 19088 | 19102 | 15 |
| 490A | 19109 | 19129 | 21 |
| 491A | 19128 | 19145 | 18 |
| 492A | 19240 | 19258 | 19 |
| 493A | 19280 | 19366 | 87 |
| 494A | 19372 | 19387 | 16 |
| 495A | 19422 | 19444 | 23 |
| 496A | 19446 | 19462 | 17 |
| 497A | 19489 | 19506 | 18 |
| 498A | 19546 | 19571 | 26 |
| 499A | 19597 | 19615 | 19 |
| 500A | 19624 | 19648 | 25 |
| 501A | 19680 | 19695 | 16 |
| 502A | 19713 | 19727 | 15 |
| 503A | 19775 | 19792 | 18 |
| 504A | 19789 | 19803 | 15 |
| 505A | 19811 | 19825 | 15 |
| 506A | 19838 | 19862 | 25 |
| 507A | 20241 | 20257 | 17 |
| 508A | 20259 | 20290 | 32 |
| 509A | 20309 | 20381 | 73 |
| 510A | 20404 | 20419 | 16 |
| 511A | 20470 | 20492 | 23 |
| 512A | 20495 | 20557 | 63 |
| 513A | 20593 | 20609 | 17 |
| 514A | 20626 | 20646 | 21 |
| 515A | 20648 | 20669 | 22 |
| 516A | 20683 | 20699 | 17 |
| 517A | 20718 | 20735 | 18 |
| 518A | 20749 | 20765 | 17 |
| 519A | 20751 | 20765 | 15 |
| 520A | 20769 | 20785 | 17 |
| 521A | 20773 | 20791 | 19 |
| 522A | 20777 | 20798 | 22 |
| 523A | 20779 | 20798 | 20 |
| 524A | 20779 | 20797 | 19 |
| 525A | 20798 | 20819 | 22 |
| 526A | 20800 | 20819 | 20 |
| 527A | 20800 | 20818 | 19 |
| 528A | 20819 | 20840 | 22 |
| 529A | 20819 | 20853 | 35 |
| 530A | 20821 | 20840 | 20 |
| 531A | 20821 | 20853 | 33 |
| 532A | 20821 | 20839 | 19 |
| 533A | 20833 | 20851 | 19 |
| 534A | 20833 | 20855 | 23 |
| 535A | 20841 | 20864 | 24 |
| 536A | 20855 | 20869 | 15 |
| 537A | 20866 | 20895 | 30 |
| 538A | 20881 | 20902 | 22 |
| 539A | 20881 | 20915 | 35 |
| 540A | 20883 | 20902 | 20 |
| 541A | 20883 | 20915 | 33 |
| 542A | 20883 | 20901 | 19 |
| 543A | 20895 | 20913 | 19 |
| 544A | 20895 | 20917 | 23 |
| 545A | 20903 | 20926 | 24 |
| 546A | 20917 | 20931 | 15 |
| 547A | 20928 | 20946 | 19 |
| 548A | 20937 | 20951 | 15 |
| 549A | 20955 | 20973 | 19 |
| 550A | 20975 | 20993 | 19 |
| 551A | 20975 | 20997 | 23 |
| 552A | 20983 | 21004 | 22 |
| 553A | 20983 | 21017 | 35 |
| 554A | 20985 | 21004 | 20 |
| 555A | 20985 | 21017 | 33 |
| 556A | 20985 | 21003 | 19 |
| 557A | 20997 | 21015 | 19 |
| 558A | 20997 | 21019 | 23 |
| 559A | 21005 | 21028 | 24 |
| 560A | 21019 | 21033 | 15 |
| 561A | 21030 | 21048 | 19 |
| 562A | 21030 | 21052 | 23 |
| 563A | 21057 | 21075 | 19 |
| 564A | 21057 | 21079 | 23 |
| 565A | 21067 | 21085 | 19 |
| 566A | 21088 | 21118 | 31 |
| 567A | 21127 | 21153 | 27 |
| 568A | 21155 | 21169 | 15 |
| 569A | 21155 | 21180 | 26 |
| 570A | 21205 | 21220 | 16 |
| 571A | 21222 | 21283 | 62 |
| 572A | 21347 | 21370 | 24 |
| 573A | 21431 | 21445 | 15 |
| 574A | 21463 | 21487 | 25 |
| 575A | 21489 | 21518 | 30 |
| 576A | 21520 | 21535 | 16 |
| 577A | 21551 | 21573 | 23 |
| 578A | 21574 | 21591 | 18 |
| 579A | 21595 | 21618 | 24 |
| 580A | 21622 | 21641 | 20 |
| 581A | 21664 | 21678 | 15 |
| 582A | 21758 | 21789 | 32 |
| 583A | 21799 | 21816 | 18 |
| 584A | 21820 | 21852 | 33 |
| 585A | 21865 | 21882 | 18 |
| 586A | 21890 | 21905 | 16 |
| 587A | 21917 | 21932 | 16 |
| 588A | 21956 | 21976 | 21 |
| 589A | 21975 | 21993 | 19 |
| 590A | 22007 | 22035 | 29 |
| 591A | 22014 | 22034 | 21 |
| 592A | 22036 | 22051 | 16 |
| 593A | 22036 | 22068 | 33 |
| 594A | 22070 | 22132 | 63 |
| 595A | 22174 | 22203 | 30 |
| 596A | 22205 | 22219 | 15 |
| 597A | 22229 | 22254 | 26 |
| 598A | 22276 | 22299 | 24 |
| 599A | 22309 | 22353 | 45 |
| 600A | 22359 | 22373 | 15 |
| 601A | 22385 | 22403 | 19 |
| 602A | 22443 | 22460 | 18 |
| 603A | 22462 | 22490 | 29 |
| 604A | 22499 | 22520 | 22 |
| 605A | 22601 | 22623 | 23 |
| 606A | 22646 | 22661 | 16 |
| 607A | 22663 | 22682 | 20 |
| 608A | 22713 | 22735 | 23 |
| 609A | 22737 | 22772 | 36 |
| 610A | 22793 | 22826 | 34 |
| 611A | 22851 | 22903 | 53 |
| 612A | 22905 | 22928 | 24 |
| 613A | 22934 | 22985 | 52 |
| 614A | 23071 | 23089 | 19 |
| 615A | 23094 | 23121 | 28 |
| 616A | 23174 | 23208 | 35 |
| 617A | 23249 | 23276 | 28 |
| 618A | 23279 | 23311 | 33 |
| 619A | 23313 | 23328 | 16 |
| 620A | 23450 | 23470 | 21 |
| 621A | 23488 | 23503 | 16 |
| 622A | 23511 | 23529 | 19 |
| 623A | 23555 | 23570 | 16 |
| 624A | 23575 | 23589 | 15 |
| 625A | 23597 | 23620 | 24 |
| 626A | 23632 | 23647 | 16 |
| 627A | 23672 | 23687 | 16 |
| 628A | 23737 | 23775 | 39 |
| 629A | 23746 | 23760 | 15 |
| 630A | 23833 | 23847 | 15 |
| 631A | 23872 | 23911 | 40 |
| 632A | 23919 | 23936 | 18 |
| 633A | 24050 | 24068 | 19 |
| 634A | 24083 | 24111 | 29 |
| 635A | 24111 | 24125 | 15 |
| 636A | 24131 | 24164 | 34 |
| 637A | 24167 | 24189 | 23 |
| 638A | 24204 | 24227 | 24 |
| 639A | 24236 | 24285 | 50 |
| 640A | 24438 | 24453 | 16 |
| 641A | 24499 | 24514 | 16 |
| 642A | 24560 | 24575 | 16 |
| 643A | 24621 | 24636 | 16 |
| 644A | 24682 | 24697 | 16 |
| 645A | 24717 | 24753 | 37 |
| 646A | 24842 | 24857 | 16 |
| 647A | 24902 | 24918 | 17 |
| 648A | 24932 | 24962 | 31 |
| 649A | 25018 | 25056 | 39 |
| 650A | 25160 | 25176 | 17 |
| 651A | 25219 | 25251 | 33 |
| 652A | 25259 | 25278 | 20 |
| 653A | 25332 | 25346 | 15 |
| 654A | 25363 | 25379 | 17 |
| 655A | 25367 | 25383 | 17 |
| 656A | 25405 | 25435 | 31 |
| 657A | 25405 | 25436 | 32 |
| 658A | 25407 | 25425 | 19 |
| 659A | 25410 | 25436 | 27 |
| 660A | 25418 | 25435 | 18 |
| 661A | 25475 | 25495 | 21 |
| 662A | 25502 | 25518 | 17 |
| 663A | 25559 | 25582 | 24 |
| 664A | 25596 | 25640 | 45 |
| 665A | 25671 | 25688 | 18 |
| 666A | 25796 | 25816 | 21 |
| 667A | 25818 | 25832 | 15 |
| 668A | 25834 | 25857 | 24 |
| 669A | 25867 | 25881 | 15 |
| 670A | 25928 | 25943 | 16 |
| 671A | 25986 | 26001 | 16 |
| 672A | 26014 | 26037 | 24 |
| 673A | 26187 | 26210 | 24 |
| 674A | 26212 | 26228 | 17 |
| 675A | 26268 | 26286 | 19 |
| 676A | 26300 | 26319 | 20 |
| 677A | 26359 | 26394 | 36 |
| 678A | 26396 | 26426 | 31 |
| 679A | 26465 | 26482 | 18 |
| 680A | 26505 | 26529 | 25 |
| 681A | 26547 | 26565 | 19 |
| 682A | 26576 | 26600 | 25 |
| 683A | 26588 | 26603 | 16 |
| 684A | 26588 | 26606 | 19 |
| 685A | 26609 | 26624 | 16 |
| 686A | 26615 | 26638 | 24 |
| 687A | 26615 | 26642 | 28 |
| 688A | 26632 | 26669 | 38 |
| 689A | 26649 | 26669 | 21 |
| 690A | 26658 | 26672 | 15 |
| 691A | 26695 | 26716 | 22 |
| 692A | 26706 | 26725 | 20 |
| 693A | 26713 | 26735 | 23 |
| 694A | 26715 | 26733 | 19 |
| 695A | 26737 | 26768 | 32 |
| 696A | 26755 | 26770 | 16 |
| 697A | 26756 | 26789 | 34 |
| 698A | 26759 | 26789 | 31 |
| 699A | 26787 | 26813 | 27 |
| 700A | 26795 | 26812 | 18 |
| 701A | 26815 | 26829 | 15 |
| 702A | 26861 | 26880 | 20 |
| 703A | 26862 | 26882 | 21 |
| 704A | 26865 | 26883 | 19 |
| 705A | 26868 | 26883 | 16 |
| 706A | 26870 | 26885 | 16 |
| 707A | 26871 | 26892 | 22 |
| 708A | 26880 | 26898 | 19 |
| 709A | 26889 | 26910 | 22 |
| 710A | 26908 | 26924 | 17 |
| 711A | 26917 | 26939 | 23 |
| 712A | 26948 | 26962 | 15 |
| 713A | 26955 | 26973 | 19 |
| 714A | 27097 | 27113 | 17 |
| 715A | 27101 | 27128 | 28 |
| 716A | 27112 | 27127 | 16 |
| 717A | 27116 | 27183 | 68 |
| 718A | 27133 | 27165 | 33 |
| 719A | 27188 | 27209 | 22 |
| 720A | 27218 | 27232 | 15 |
| 721A | 27218 | 27234 | 17 |
| 722A | 27235 | 27253 | 19 |
| 723A | 27237 | 27253 | 17 |
| 724A | 27237 | 27251 | 15 |
| 725A | 27237 | 27252 | 16 |
| 726A | 27238 | 27254 | 17 |
| 727A | 27238 | 27252 | 15 |
| 728A | 27238 | 27253 | 16 |
| 729A | 27239 | 27255 | 17 |
| 730A | 27239 | 27253 | 15 |
| 731A | 27239 | 27254 | 16 |
| 732A | 27240 | 27254 | 15 |
| 733A | 27240 | 27255 | 16 |
| 734A | 27241 | 27255 | 15 |
| 735A | 27269 | 27320 | 52 |
| 736A | 27281 | 27297 | 17 |
| 737A | 27286 | 27307 | 22 |
| 738A | 27340 | 27358 | 19 |
| 739A | 27360 | 27404 | 45 |
| 740A | 27411 | 27438 | 28 |
| 741A | 27458 | 27474 | 17 |
| 742A | 27531 | 27572 | 42 |
| 743A | 27575 | 27600 | 26 |
| 744A | 27602 | 27616 | 15 |
| 745A | 27618 | 27637 | 20 |
| 746A | 27670 | 27684 | 15 |
| 747A | 27707 | 27721 | 15 |
| 748A | 27723 | 27747 | 25 |
| 749A | 27772 | 27816 | 45 |
| 750A | 27772 | 27790 | 19 |
| 751A | 27829 | 27847 | 19 |
| 752A | 27850 | 27868 | 19 |
| 753A | 27870 | 27905 | 36 |
| 754A | 27927 | 27942 | 16 |
| 755A | 27963 | 27987 | 25 |
| 756A | 27989 | 28083 | 95 |
| 757A | 28085 | 28103 | 19 |
| 758A | 28120 | 28138 | 19 |
| 759A | 28167 | 28188 | 22 |
| 760A | 28190 | 28207 | 18 |
| 761A | 28209 | 28231 | 23 |
| 762A | 28234 | 28250 | 17 |
| 763A | 28260 | 28303 | 44 |
| 764A | 28427 | 28444 | 18 |
| 765A | 28446 | 28462 | 17 |
| 766A | 28464 | 28484 | 21 |
| 767A | 28503 | 28519 | 17 |
| 768A | 28521 | 28536 | 16 |
| 769A | 28538 | 28565 | 28 |
| 770A | 28595 | 28612 | 18 |
| 771A | 28694 | 28709 | 16 |
| 772A | 28701 | 28715 | 15 |
| 773A | 28715 | 28751 | 37 |
| 774A | 28801 | 28825 | 25 |
| 775A | 28832 | 28846 | 15 |
| 776A | 28846 | 28870 | 25 |
| 777A | 28878 | 28893 | 16 |
| 778A | 28895 | 28911 | 17 |
| 779A | 28938 | 28961 | 24 |
| 780A | 29010 | 29025 | 16 |
| 781A | 29057 | 29072 | 16 |
| 782A | 29119 | 29134 | 16 |
| 783A | 29179 | 29193 | 15 |
| 784A | 29235 | 29256 | 22 |
| 785A | 29330 | 29349 | 20 |
| 786A | 29367 | 29381 | 15 |
| 787A | 29530 | 29556 | 27 |
| 788A | 29587 | 29605 | 19 |
| 789A | 29652 | 29692 | 41 |
| 790A | 29695 | 29710 | 16 |
| 791A | 29722 | 29742 | 21 |
| 792A | 29743 | 29768 | 26 |
| 793A | 29770 | 29797 | 28 |
| 794A | 29818 | 29836 | 19 |
| 795A | 29838 | 29873 | 36 |
| 796A | 29875 | 29946 | 72 |
| 797A | 29948 | 29983 | 36 |
| 798A | 30028 | 30048 | 21 |
| 799A | 30046 | 30060 | 15 |
| 800A | 30051 | 30067 | 17 |
| 801A | 30069 | 30090 | 22 |
| 802A | 30093 | 30107 | 15 |
| 803A | 30116 | 30136 | 21 |
| 804A | 30138 | 30202 | 65 |
| 805A | 30220 | 30262 | 43 |
| 806A | 30303 | 30320 | 18 |
| 807A | 30349 | 30372 | 24 |
| 808A | 30387 | 30418 | 32 |
| 809A | 30417 | 30441 | 25 |
| 810A | 30476 | 30516 | 41 |
| 811A | 30524 | 30576 | 53 |
| 812A | 30602 | 30628 | 27 |
| 813A | 30658 | 30680 | 23 |
| 814A | 30682 | 30747 | 66 |
| 815A | 30749 | 30799 | 51 |
| 816A | 30801 | 30821 | 21 |
| 817A | 30823 | 30844 | 22 |
| 818A | 30908 | 30922 | 15 |
| 819A | 30924 | 30980 | 57 |
| 820A | 31027 | 31045 | 19 |
| 821A | 31047 | 31080 | 34 |
| 822A | 31086 | 31113 | 28 |
| 823A | 31128 | 31146 | 19 |
| 824A | 31150 | 31164 | 15 |
| 825A | 31166 | 31193 | 28 |
| 826A | 31229 | 31271 | 43 |
| 827A | 31276 | 31310 | 35 |
| 828A | 31312 | 31333 | 22 |
| 829A | 31400 | 31417 | 18 |
| 830A | 31419 | 31433 | 15 |
| 831A | 31456 | 31470 | 15 |
| 832A | 31517 | 31569 | 53 |
| 833A | 31578 | 31599 | 22 |
| 834A | 31661 | 31689 | 29 |
| 835A | 31706 | 31739 | 34 |
| 836A | 31741 | 31763 | 23 |
| 837A | 31765 | 31805 | 41 |
| 838A | 31807 | 31855 | 49 |
| 839A | 31819 | 31834 | 16 |
| 840A | 31851 | 31866 | 16 |
| 841A | 31857 | 31872 | 16 |
| 842A | 31938 | 31984 | 47 |
| 843A | 31986 | 32032 | 47 |
| 844A | 32034 | 32071 | 38 |
| 845A | 32082 | 32097 | 16 |
| 846A | 32124 | 32151 | 28 |
| 847A | 32197 | 32216 | 20 |
| 848A | 32233 | 32262 | 30 |
| 849A | 32264 | 32289 | 26 |
| 850A | 32306 | 32325 | 20 |
| 851A | 32357 | 32408 | 52 |
| 852A | 32410 | 32459 | 50 |
| 853A | 32474 | 32492 | 19 |
| 854A | 32494 | 32508 | 15 |
| 855A | 32527 | 32543 | 17 |
| 856A | 32545 | 32560 | 16 |
| 857A | 32570 | 32636 | 67 |
| 858A | 32697 | 32713 | 17 |
| 859A | 32744 | 32765 | 22 |
| 860A | 32801 | 32823 | 23 |
| 861A | 32865 | 32892 | 28 |
| 862A | 32944 | 32959 | 16 |
| 863A | 32962 | 32985 | 24 |
| 864A | 32998 | 33104 | 107 |
| 865A | 33126 | 33140 | 15 |
| 866A | 33142 | 33194 | 53 |
| 867A | 33213 | 33252 | 40 |
| 868A | 33277 | 33298 | 22 |
| 869A | 33318 | 33365 | 48 |
| 870A | 33375 | 33390 | 16 |
| 871A | 33402 | 33417 | 16 |
| 872A | 33419 | 33443 | 25 |
| 873A | 33456 | 33488 | 33 |
| 874A | 33509 | 33542 | 34 |
| 875A | 33562 | 33583 | 22 |
| 876A | 33607 | 33622 | 16 |
| 877A | 33655 | 33700 | 46 |
| 878A | 33704 | 33720 | 17 |
| 879A | 33735 | 33753 | 19 |
| 880A | 33755 | 33780 | 26 |
| 881A | 33806 | 33820 | 15 |
| 882A | 33829 | 33845 | 17 |
| 883A | 33916 | 33962 | 47 |
| 884A | 33964 | 33982 | 19 |
| 885A | 33989 | 34026 | 38 |
| 886A | 34028 | 34072 | 45 |
| 887A | 34089 | 34104 | 16 |
| 888A | 34113 | 34130 | 18 |
| 889A | 34141 | 34158 | 18 |
| 890A | 34281 | 34309 | 29 |
| 891A | 34377 | 34407 | 31 |
| 892A | 34423 | 34498 | 76 |
| 893A | 34507 | 34521 | 15 |
| 894A | 34524 | 34545 | 22 |
| 895A | 34552 | 34596 | 45 |
| 896A | 34688 | 34703 | 16 |
| 897A | 34742 | 34759 | 18 |
| 898A | 34770 | 34798 | 29 |
| 899A | 34860 | 34882 | 23 |
| 900A | 34919 | 34938 | 20 |
| 901A | 34950 | 34988 | 39 |
| 902A | 34990 | 35012 | 23 |
| 903A | 35022 | 35048 | 27 |
| 904A | 35063 | 35182 | 120 |
| 905A | 35184 | 35210 | 27 |
| 906A | 35222 | 35241 | 20 |
| 907A | 35245 | 35275 | 31 |
| 908A | 35277 | 35297 | 21 |
| 909A | 35319 | 35355 | 37 |
| 910A | 35367 | 35397 | 31 |
| 911A | 35433 | 35457 | 25 |
| 912A | 35461 | 35486 | 26 |
| 913A | 35490 | 35509 | 20 |
| 914A | 35546 | 35560 | 15 |
| 915A | 35573 | 35593 | 21 |
| 916A | 35597 | 35613 | 17 |
| 917A | 35968 | 35999 | 32 |
| 918A | 35997 | 36011 | 15 |
| 919A | 36037 | 36051 | 15 |
| 920A | 36097 | 36118 | 22 |
| 921A | 36117 | 36132 | 16 |
| 922A | 36278 | 36295 | 18 |
| 923A | 36350 | 36364 | 15 |
| 924A | 36366 | 36392 | 27 |
| 925A | 36433 | 36458 | 26 |
| 926A | 36460 | 36483 | 24 |
| 927A | 36530 | 36547 | 18 |
| 928A | 36549 | 36566 | 18 |
| 929A | 36600 | 36625 | 26 |
| 930A | 36627 | 36665 | 39 |
| 931A | 36759 | 36774 | 16 |
| 932A | 36765 | 36782 | 18 |
| 933A | 36815 | 36850 | 36 |
| 934A | 36873 | 36891 | 19 |
| 935A | 36894 | 36934 | 41 |
| 936A | 36969 | 36994 | 26 |
| 937A | 36996 | 37016 | 21 |
| 938A | 37023 | 37040 | 18 |
| 939A | 37093 | 37112 | 20 |
| 940A | 37118 | 37142 | 25 |
| 941A | 37144 | 37163 | 20 |
| 942A | 37242 | 37324 | 83 |
| 943A | 37352 | 37368 | 17 |
| 944A | 37370 | 37389 | 20 |
| 945A | 37391 | 37419 | 29 |
| 946A | 37421 | 37438 | 18 |
| 947A | 37444 | 37491 | 48 |
| 948A | 37511 | 37538 | 28 |
| 949A | 37567 | 37614 | 48 |
| 950A | 37636 | 37680 | 45 |
| 951A | 37723 | 37765 | 43 |
| 952A | 37773 | 37801 | 29 |
| 953A | 37803 | 37822 | 20 |
| 954A | 37824 | 37853 | 30 |
| 955A | 37855 | 37887 | 33 |
| 956A | 37889 | 37908 | 20 |
| 957A | 37920 | 37939 | 20 |
| 958A | 37988 | 38020 | 33 |
| 959A | 38022 | 38049 | 28 |

In some embodiments, the oligonucleotide comprises a contiguous nucleotide sequence of 16 to 20, such as 15 to 22, nucleotides in length with at least 90% complementary, such as 100% complementarity, to a corresponding target sequence present in SEQ ID NO: 1, wherein the target sequence is selected from the group consisting of SEQ ID NO: 3 to 21 (table 4) or region B1 to B28 in Table 5B.

TABLE 5B
Regions of SEQ ID NO 1 which may be
targeted using an oligonucleotide of the invention

| | Position in SEQ ID NO 1 | | |
|---|---|---|---|
| Reg. B | from | to | Length |
| 1 | 8295 | 8312 | 17 |
| 2 | 8684 | 8704 | 20 |
| 3 | 9668 | 9684 | 16 |
| 4 | 9669 | 9684 | 15 |
| 5 | 9722 | 9741 | 19 |
| 6 | 9723 | 9741 | 18 |
| 7 | 9724 | 9742 | 18 |
| 8 | 10921 | 10937 | 16 |
| 9 | 11483 | 11503 | 20 |
| 10 | 11512 | 11531 | 19 |
| 11 | 11622 | 11641 | 19 |
| 12 | 11753 | 11773 | 20 |
| 13 | 11755 | 11772 | 17 |
| 14 | 11756 | 11776 | 20 |
| 15 | 11757 | 11776 | 19 |
| 16 | 11758 | 11778 | 20 |
| 17 | 12868 | 11885 | 17 |
| 18 | 13234 | 13252 | 18 |
| 19 | 13551 | 13569 | 18 |
| 20 | 14786 | 14804 | 18 |
| 21 | 18085 | 18101 | 16 |
| 22 | 22425 | 22441 | 16 |
| 23 | 33030 | 33048 | 18 |
| 24 | 35103 | 35123 | 20 |
| 25 | 35371 | 35390 | 19 |
| 26 | 35636 | 35655 | 19 |
| 27 | 35638 | 35654 | 16 |
| 28 | 36915 | 36931 | 16 |

In some embodiments, the oligonucleotide of the invention comprises or consists of 12 to 60 nucleotides in length, such as from 13 to 50, such as from 14 to 35, such as 15 to 30, such as from 16 to 20 contiguous nucleotides in length. In a preferred embodiment, the oligonucleotide comprises or consists of 15, 16, 17, 18, 19 or 20 nucleotides in length.

In some embodiments, the contiguous nucleotide sequence of the oligonucleotide which is complementary to the target nucleic acids comprises or consists of 12 to 30, such as from 13 to 25, such as from 15 to 23, such as from 16 to 22, contiguous nucleotides in length.

In some embodiments, the contiguous nucleotide sequence of the siRNA or shRNA which is complementary to the target nucleic acids comprises or consists of 18 to 28, such as from 19 to 26, such as from 20 to 24, such as from 21 to 23, contiguous nucleotides in length.

In some embodiments, the contiguous nucleotide sequence of the single stranded antisense oligonucleotide which is complementary to the target nucleic acids comprises or consists of 12 to 22, such as from 14 to 20, such as from 16 to 20, such as from 15 to 21, such as from 15 to 18, such as from 16 to 18, such as from 16 to 17 contiguous nucleotides in length.

In some embodiments, the oligonucleotide or contiguous nucleotide sequence comprises or consists of a sequence selected from the group consisting of sequences listed in table 6 (Materials and Method section).

In some embodiments, the oligonucleotide or contiguous nucleotide sequence comprises or consists of 10 to 30 nucleotides in length with at least 90% identity, preferably 100% identity, to a sequence selected from the group consisting of SEQ ID NO: 22 to 237 (see motif sequences listed in table 6). In a particular embodiment the oligonucleotide or contiguous nucleotide sequence is selected from SEQ ID NO: 22; 23; 24; 25; 26; 27; 28; 29; 32; 35; 36; 37; 38; 39; 40; 41; 42; 42; 42; 43; 43; 46; 49; 83; 109; 130; 203; and 232.

It is understood that the contiguous oligonucleotide sequence (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The oligonucleotide of the invention may be designed with modified nucleosides and RNA nucleosides (in particular for siRNA and shRNA molecules) or DNA nucleosides (in particular for single stranded antisense oligonucleotides). Advantageously, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 2 to 3 internucleoside linkages at the 5′ or 3′ end of the oligonucleotide are phosphorothioate internucleoside linkages. For single stranded antisense oligonucleotides it is advantageous if at least 75%, such as all, the internucleoside linkages within the contiguous nucleotide sequence are phosphorothioate internucleoside linkages. In some embodiments all the internucleotide linkages in the contiguous sequence of the single stranded antisense oligonucleotide are phosphorothioate linkages.

In some embodiments, the oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In an embodiment of the invention the oligonucleotide of the invention is capable of recruiting RNase H.

In the current invention an advantageous structural design is a gapmer design as described in the “Definitions” section under for example “Gapmer”, “LNA Gapmer” and “MOE gapmer”. In the present invention it is advantageous if the antisense oligonucleotide of the invention is a gapmer with an F-G-F′ design. In some embodiments the gapmer is an LNA gapmer with uniform flanks.

In some embodiments of the invention the LNA gapmer is selected from the following uniform flank designs: 2-12-3, 4-14-2, 3-10-3, 3-9-3, 2-15-2, 2-12-4, 1-13-2, 3-13-2, 4-13-2, 2-12-2, 3-12-2, 3-15-2, 3-14-2, 3-13-3, 2-14-4, 3-12-3, 1-14-3, 3-14-3, 2-14-3, 2-15-3, 3-11-3, 1-12-3, 1-11-4, 1-13-2, 2-13-2, 2-16-2, 1-14-2, 1-17-3 and 1-18-2.

Table 6 (Materials and Method section) lists preferred designs of each motif sequence.

In all instances the F-G-F′ design may further include region D′ and/or D″ as described in the “Definitions” section under “Region D′or D” in an oligonucleotide”. In some embodiments the oligonucleotide of the invention has 1, 2 or 3 phosphodiester linked nucleoside units, such as DNA units, at the 5′ or 3′ end of the gapmer region. In some embodiments the oligonucleotide of the invention consists of two 5′ phosphodiester linked DNA nucleosides followed by a F-G-F′ gapmer region as defined in the “Definitions” section. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties.

For some embodiments of the invention, the oligonucleotide is selected from the group of oligonucleotide compounds with CMP-ID-NO: 22_1; 23_1; 24_1; 25_1; 26_1; 27_1; 28_1; 29_1; 30_1; 31_1; 32_1; 33_1; 34_1; 35_1; 36_1; 37_1; 38_1; 39_1; 40_1; 41_1; 42_1; 42_2; 42_3; 43_1; 43_2; 44_1; 45_1; 46_1; 47_1; 48_1; 49_1; 130_1; 109_1; 83_1; 203_1 and 232_1 (see Table 6).

### Conjugates

Since HBV infection primarily affects the hepatocytes in the liver it is advantageous to conjugate the RTEL1 inhibitor to a conjugate moiety that will increase the delivery of the inhibitor to the liver compared to the unconjugated inhibitor. In one embodiment liver targeting moieties are selected from moieties comprising cholesterol or other lipids or conjugate moieties capable of binding to the asialoglycoprotein receptor (ASGPR).

In some embodiments, the invention provides a conjugate comprising a nucleic acid molecule of the invention covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S.T. and Drickamer, K. JB.C. 1996, 271, 6686) or are readily determined using methods typical in the art.

In one embodiment the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GalNAc).

To generate the ASGPR conjugate moiety the ASPGR targeting moieties (preferably GalNAc) can be attached to a conjugate scaffold. Generally, the ASPGR targeting moieties can be at the same end of the scaffold. In one embodiment, the conjugate moiety consists of two to four terminal GalNAc moieties linked to a spacer which links each GalNAc moiety to a brancher molecule that can be conjugated to the antisense oligonucleotide.

In a further embodiment, the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GalNAc) moieties.

GalNAc conjugate moieties can include, for example, those described in WO 2014/179620 and WO 2016/055601 and PCT/EP2017/059080 (hereby incorporated by reference), as well as small peptides with GalNAc moieties attached such as Tyr-Glu-Glu-(aminohexyl GalNAc)3 (YEE(ahGalINAc)3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GalNAc conjugate moiety, may be attached to the 3′- or 5′-end of the oligonucleotide using methods known in the art. In one embodiment the ASGPR conjugate moiety is linked to the 5′-end of the oligonucleotide.

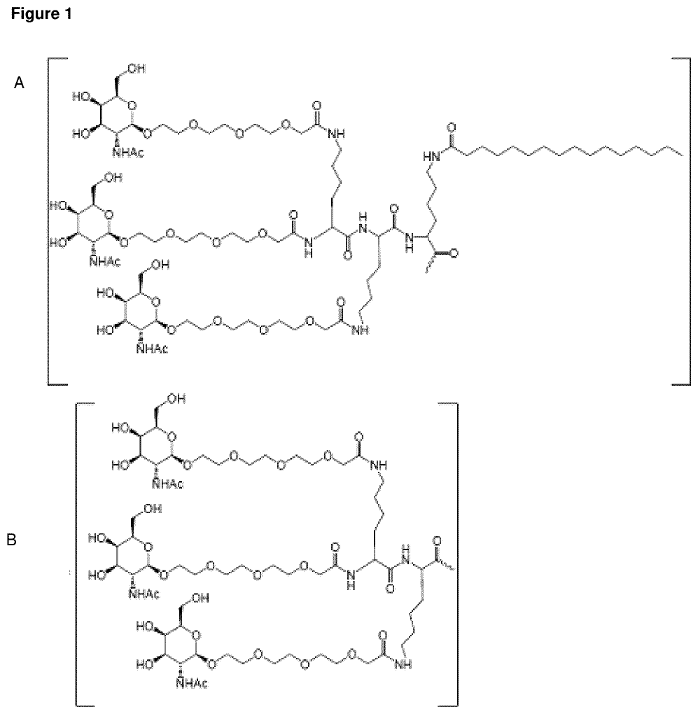

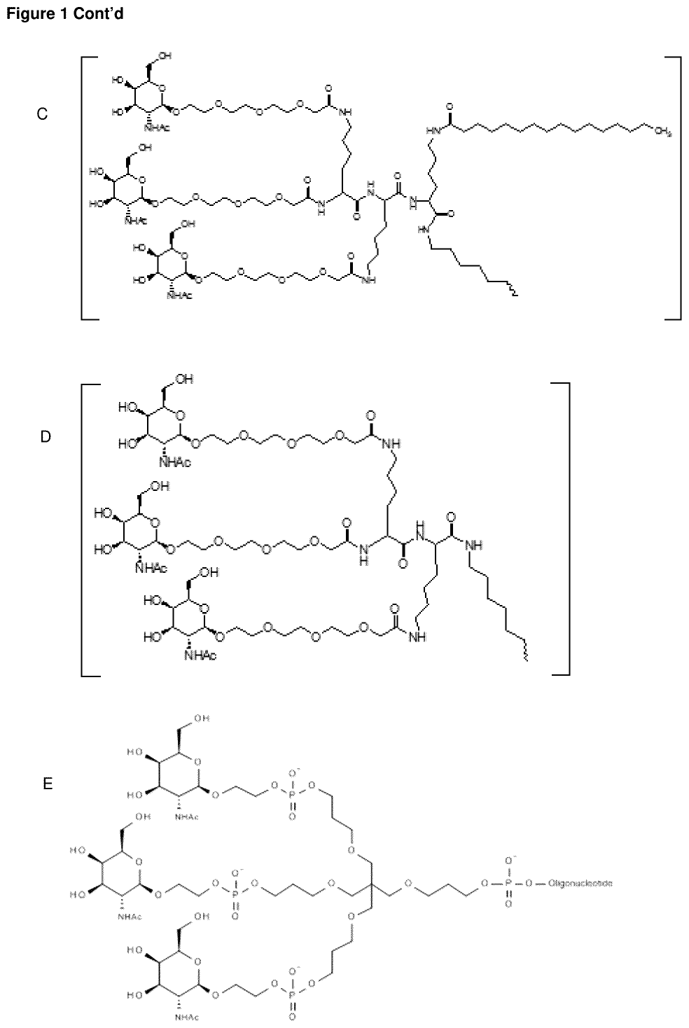

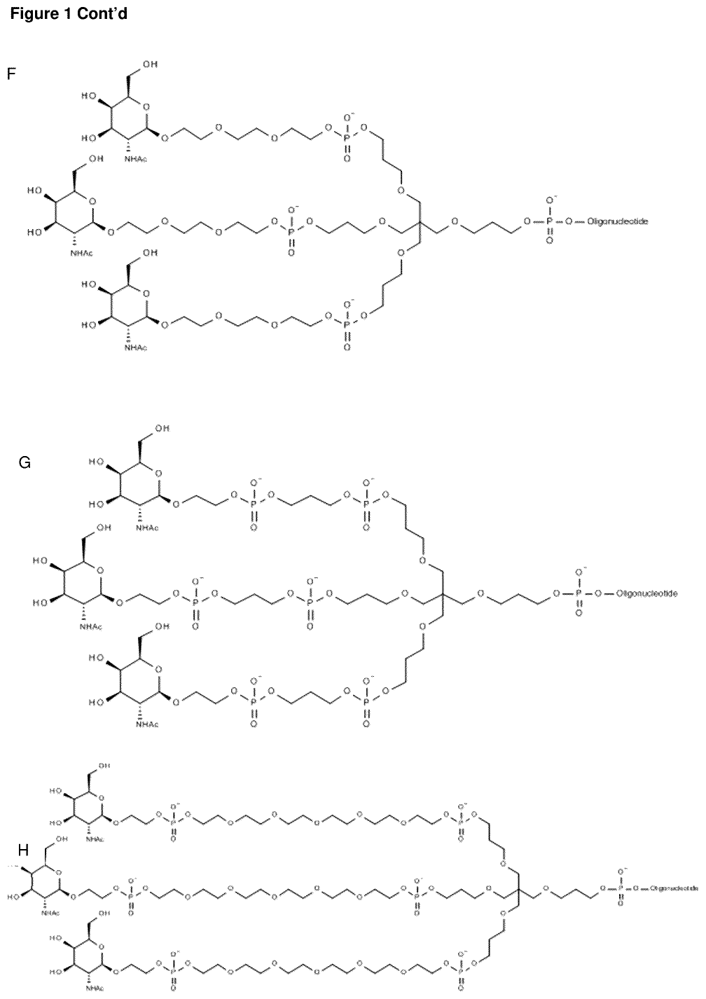

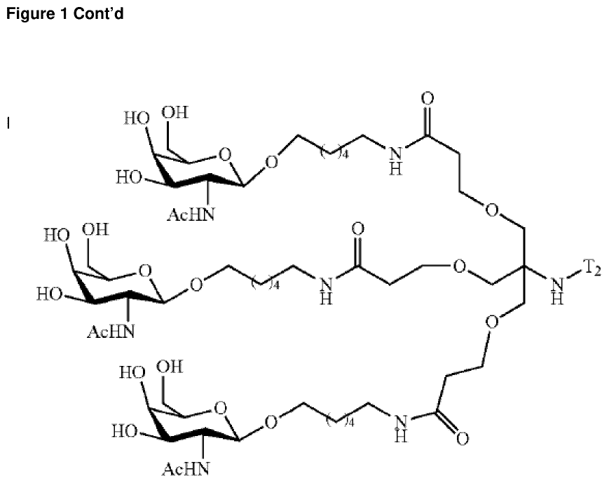

In one embodiment the conjugate moiety is a tri-valent N-acetylgalactosamine (GalNAc), such as those shown in FIG. 1, in particular as shown in FIG. 1D.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention provides a pharmaceutically acceptable salt of the antisense oligonucleotide or a conjugate thereof. In a preferred embodiment, the pharmaceutically acceptable salt is a sodium or a potassium salt.

### Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

In some embodiments, the oligonucleotide or oligonucleotide conjugates of the invention, or pharmaceutically acceptable salt thereof is in a solid form, such as a powder, such as a lyophilized powder.

Compounds, oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular, with respect to oligonucleotide conjugates the conjugate moiety is cleaved off the oligonucleotide once the prodrug is delivered to the site of action, e.g. the target cell.

### Administration

The compounds, oligonucleotides, oligonucleotide conjugates or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The invention also provides for the use of the oligonucleotide or oligonucleotide conjugate of the invention as described for the manufacture of a medicament wherein the medicament is in a dosage form for subcutaneous administration.

### Combination Therapies

In some embodiments the inhibitor of the present invention, such as the compound, oligonucleotide, oligonucleotide conjugate or pharmaceutical composition of the invention is for use in a combination treatment with another therapeutic agent. The therapeutic agent can for example be the standard of care for the diseases or disorders described above.

By way of example, the oligomer or the oligomer conjugate of the present invention may be used in combination with other actives, such as oligonucleotide-based antivirals—such as sequence specific oligonucleotide-based antivirals—acting either through antisense (including other LNA oligomers), siRNAs (such as ARC520), aptamers, morpholinos or any other antiviral, nucleotide sequence-dependent mode of action.

By way of further example, the oligomer or the oligomer conjugate of the present invention may be used in combination with other actives, such as immune stimulatory antiviral compounds, such as interferon (e.g. pegylated interferon alpha), TLR7 agonists (e.g. GS-9620), or therapeutic vaccines.

By way of further example, the oligomer or the oligomer conjugate of the present invention may be used in combination with other actives, such as small molecules, with antiviral activity. These other actives could be, for example, nucleoside/nucleotide inhibitors (eg entecavir or tenofovir disoproxil fumarate), encapsidation inhibitors, entry inhibitors (eg Myrcludex B).

In certain embodiments, the additional therapeutic agent may be an HBV agent, a Hepatitis C virus (HCV) agent, a chemotherapeutic agent, an antibiotic, an analgesic, a nonsteroidal anti-inflammatory (NSAID) agent, an antifungal agent, an antiparasitic agent, an anti-nausea agent, an anti-diarrheal agent, or an immunosuppressant agent.

In particular, related embodiments, the additional HBV agent may be interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In other particular related embodiments, the additional HCV agent may be interferon alpha-2b, interferon alpha-2a, and interferon alphacon-1 (pegylated and unpegylated); ribavirin; pegasys; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

### Applications

The oligonucleotides of the invention may be utilized as research reagents for, for example, diagnostics, therapeutics and prophylaxis.

In research, such oligonucleotides may be used to specifically modulate the synthesis of RTEL1 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the oligonucleotides of the invention in research or diagnostics the target nucleic acid may be a cDNA or a synthetic nucleic acid derived from DNA or RNA.

Also encompassed by the present invention is an in vivo or in vitro method for modulating RTEL1 expression in a target cell which is expressing RTEL1, said method comprising administering an oligonucleotide, conjugate compound or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is present in in the liver. The target cell may be a hepatocyte.

One aspect of the present invention is related the oligonucleotides, conjugate compounds or pharmaceutical compositions of the invention for use as a medicament.

In an aspect of the invention the oligonucleotides, conjugate compound or pharmaceutical composition of the invention is capable of reducing the cccDNA level in the infected cells and therefore inhibiting HBV infection. In particular, the antisense oligonucleotide is capable of affecting one or more of the following parameters i) reducing cccDNA and/or ii) reducing pgRNA and/or iii) reducing HBV DNA and/or iv) reducing HBV viral antigens in an infected cell.

For example, nucleic acid molecule that inhibits HBV infection may reduce i) the cccDNA levels in an infected cell by at least 40% such as 50%, 60%, 70%, 80%, or 90% reduction compared to controls; or ii) the level of pgRNA by at least 40% such as 50%, 60%, 70%, 80%, or 90% reduction compared to controls. The controls may be untreated cells or animals, or cells or animals treated with an appropriate control.

Inhibition of HBV infection may be measured in vitro using HBV infected primary human hepatocytes or in vivo using humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of intracellular cccDNA or HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

Due to the reduction of RTEL1 levels the oligonucleotides, conjugate compounds or pharmaceutical compositions of the present invention can be used to inhibit development of or in the treatment of HBV infection. In particular, the destabilization and reduction of the cccDNA, the oligonucleotides, conjugate compounds or pharmaceutical compositions of the present invention more efficiently inhibits development of or treats a chronic HBV infection as compared to a compound that only reduces secretion of HBsAg.

Accordingly, one aspect of the present invention is related to use of the oligonucleotide, conjugate compounds or pharmaceutical compositions of the invention to reduce cccDNA and/or pgRNA in an HBV infected individual.

A further aspect of the invention relates to the use of the oligonucleotides, conjugate compounds or pharmaceutical compositions of the invention to inhibit development of or treat a chronic HBV infection.

A further aspect of the invention relates to the use of the oligonucleotides, conjugate compounds or pharmaceutical compositions of the invention to reduce the infectiousness of a HBV infected person. In a particular aspect of the invention, the oligonucleotides, conjugate compounds or pharmaceutical compositions of the invention inhibits development of a chronic HBV infection.

The subject to be treated with the oligonucleotides, conjugate compounds or pharmaceutical compositions of the invention (or which prophylactically receives antisense oligonucleotides, conjugate compounds or pharmaceutical compositions of the present invention) is preferably a human, more preferably a human patient who is HBsAg positive and/or HBeAg positive, even more preferably a human patient that is HBsAg positive and HBeAg positive.

Accordingly, the present invention relates to a method of treating a HBV infection, wherein the method comprises administering an effective amount of the oligonucleotides, conjugate compounds or pharmaceutical compositions of the invention. The present invention further relates to a method of preventing liver cirrhosis and hepatocellular carcinoma caused by a chronic HBV infection.

The invention also provides for the use of an oligonucleotide, a conjugate compound or a pharmaceutical composition of the invention for the manufacture of a medicament, in particular a medicament for use in the treatment of HBV infection or chronic HBV infection or reduction of the infectiousness of a HBV infected person. In preferred embodiments the medicament is manufactured in a dosage form for subcutaneous administration.

The invention also provides for the use of an oligonucleotide, a conjugate compound, the pharmaceutical composition of the invention for the manufacture of a medicament wherein the medicament is in a dosage form for intravenous administration.

The oligonucleotide, conjugate or the pharmaceutical composition of the invention may be used in a combination therapy. For example, oligonucleotide, conjugate or the pharmaceutical composition of the invention may be combined with other anti-HBV agents such as interferon alpha-2b, interferon alpha-2a, and interferon alphacon-1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697, WO 2014/179629 and WO2017/216390), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Embodiments of the Invention

The following embodiments of the present invention may be used in combination with any other embodiments described herein.
1. A RTEL1 inhibitor for use in the in the treatment and/or prevention of Hepatitis B virus (HBV) infection.
2. The RTEL1 inhibitor for the use of embodiment 1, wherein the RTEL1 inhibitor is administered in an effective amount.
3. The RTEL1 inhibitor for the use of embodiment 1 or 2, wherein the HBV infection is a chronic infection.
4. The RTEL1 inhibitor for the use of embodiments 1 to 3, wherein the RTEL1 inhibitor is capable of reducing cccDNA and/or pgRNA in an infected cell.
5. The RTEL1 inhibitor for the use of any one of embodiments 1 to 4, wherein the RTEL1 inhibitor prevents or reduces the binding of RTEL1 to DNA, such as cccDNA.
6. RTEL1 inhibitor for the use of embodiment 5, wherein said inhibitor is a small molecule that specifically binds to RTEL1 protein, wherein said inhibitor prevents or reduces binding of RTEL1 protein to cccDNA.
7. The RTEL1 inhibitor for the use of any one of embodiments 1 to 5, wherein said inhibitor is an oligonucleotide of 12-60 nucleotides in length comprising or consisting of a contiguous nucleotide sequence of at least 10 nucleotides in length which is at least 90% complementary to a mammalian RTEL1 target nucleic acid.
8. The RTEL1 inhibitor for the use of embodiment 7, which is capable of reducing the level of the RTEL1 target nucleic acid.
9. The RTEL1 inhibitor for the use of embodiment 7 or 8, wherein the target nucleic acid is RNA.
10. The RTEL1 inhibitor for the use of embodiment 9, wherein the RNA is pre-mRNA.
11. The RTEL1 inhibitor for the use of any one of embodiments 7 to 10, wherein the oligonucleotide is selected from an antisense oligonucleotide, siRNA or shRNA.
12. The RTEL1 inhibitor for the use of embodiments 11, wherein the oligonucleotide is a single stranded antisense oligonucleotide or a double stranded siRNA.
13. The RTEL1 inhibitor for the use of any one of embodiments 7 to 12, wherein the mammalian RTEL1 target nucleic acid is selected from SEQ ID NO: 1 or 2.
14. The RTEL1 inhibitor for the use of any one of embodiments 7 to 12, wherein the contiguous nucleotide sequence of the oligonucleotide is at least 98% complementarity to the target nucleic acid of SEQ ID NO: 1 and SEQ ID NO: 2.
15. The RTEL1 inhibitor for the use of any one of embodiments 1 to 14, wherein the cccDNA in an HBV infected cell is reduced by at least 50%, such as 60%, such as 70%, such as 80%, such 90%, such as 95%, such as 100%, when compared to a control.
16. The oligonucleotide for the use of any one of embodiments 7 to 15, wherein the RTEL1 mRNA is reduced by at least 50%, such as 60%, such as 70%, such as 80%, such as 90%, such as 95%, such as 100%, when compared to a control.
17. An oligonucleotide of 12 to 60 nucleotides in length which comprises or consists of a contiguous nucleotide sequence of 12 to 30 nucleotides in length wherein the contiguous nucleotide sequence is at least 90% complementary, such as 95%, such as 98%, such as fully complementarity, to a mammalian RTEL1 target nucleic acid.
18. The oligonucleotide of embodiment 17, wherein the oligonucleotide is chemically produced.
19. The oligonucleotide of embodiment 17 or 18, wherein the mammalian RTEL1 target nucleic acid is selected from SEQ ID NO: 1 or 2.
20. The oligonucleotide embodiment 17 or 18, wherein the contiguous nucleotide sequence is at least 98% complementarity to the target nucleic acid of SEQ ID NO: 1 and SEQ ID NO: 2.
21. The oligonucleotide of any one of embodiments 17 to 20, wherein the oligonucleotide is 12 to 30 nucleotides in length.
22. The oligonucleotide of any one of embodiments 17 to 21, wherein the oligonucleotide is a RNAi molecule, such as a double stranded siRNA or shRNA
23. The oligonucleotide of any one of embodiments 17 to 21, wherein the oligonucleotide is a single stranded antisense oligonucleotide.
24. The oligonucleotide of any one of embodiments 17 to 23, wherein contiguous nucleotide sequence is complementary to a target sequence selected from SEQ ID NO: 3 to 21 (table 4).
25. The oligonucleotide of embodiment 17 to 24, which is capable of hybridizing to a target nucleic acid of SEQ ID NO: 1 and SEQ ID NO: 2 with a ΔG° below −15 kcal.
26. The oligonucleotide of any one of embodiments 17 to 25, wherein the contiguous nucleotide sequence comprises or consists of at least 14 contiguous nucleotides, particularly 15, 16, 17, 18, 19, 20, 21 or 22 contiguous nucleotides.
27. The oligonucleotide of any one of embodiments 17 to 25, wherein the contiguous nucleotide sequence comprises or consists of from 14 to 22 nucleotides.
28. The oligonucleotide of embodiment 27, wherein the contiguous nucleotide sequence comprises or consists of from 16 to 20 nucleotides.
29. The oligonucleotide of any one of embodiments 17 to 28, wherein the oligonucleotide comprises or consists of 14 to 25 nucleotides in length.
30. The oligonucleotide of embodiment 29, wherein the oligonucleotide comprises or consists of 16 to 22 nucleotides in length.
31. The oligonucleotide of any one of embodiment 17 to 30, wherein the oligonucleotide comprises a sequence selected from SEQ ID NO: 22-237.
32. The oligonucleotide of any one of embodiments 17 to 31, wherein the contiguous nucleotide sequence has zero to three mismatches compared to the target nucleic acids it is complementary to.
33. The oligonucleotide of embodiment 32, wherein the contiguous nucleotide sequence has one mismatch compared to the target nucleic acids.
34. The oligonucleotide of embodiment 32, wherein the contiguous nucleotide sequence has two mismatches compared to the target nucleic acids.
35. The oligonucleotide of embodiment 32, wherein the contiguous nucleotide sequence is fully complementary to both target nucleic acid sequences.
36. The oligonucleotide of embodiment 17 to 35, comprising one or more modified nucleosides.
37. The oligonucleotide of embodiment 36, wherein the one or more modified nucleoside is a high-affinity modified nucleosides.
38. The oligonucleotide of embodiment 36 or 37, wherein the one or more modified nucleoside is a 2′ sugar modified nucleoside.
39. The oligonucleotide of embodiment 38, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.
40. The oligonucleotide of embodiment 36-39, wherein the one or more modified nucleoside is a LNA nucleoside.
41. The oligonucleotide of embodiment 40, wherein the modified LNA nucleoside is selected from oxy-LNA, amino-LNA, thio-LNA, cET, and ENA.
42. The oligonucleotide of embodiment 40 or 41, wherein the modified LNA nucleoside is oxy-LNA with the following 2′-4′ bridge-O—CH₂—.
43. The oligonucleotide of embodiment 42, wherein the oxy-LNA is beta-D-oxy-LNA.
44. The oligonucleotide of embodiment 40 or 41, wherein the modified LNA nucleoside is cET with the following 2′-4′ bridge-O—CH(CH₃)—.
45. The oligonucleotide of embodiment 44, wherein the cET is (S)cET, i.e. 6′(S)methyl-beta-D-oxy-LNA.
46. The oligonucleotide of embodiment 40 or 41, wherein the LNA is ENA, with the following 2′-4′ bridge-O—CH₂—CH₂—.
47. The oligonucleotide of any one of embodiments 17 to 46, wherein the oligonucleotide comprises at least one modified internucleoside linkage.
48. The oligonucleotide of embodiment 47, wherein the modified internucleoside linkage is nuclease resistant.
49. The oligonucleotide of embodiment 47 or 48, wherein the modified internucleoside linkages is a phosphorothioate internucleoside linkages.
50. The oligonucleotide any one of embodiments 17 to 49, wherein the oligonucleotide is an antisense oligonucleotide capable of recruiting RNase H.
51. The antisense oligonucleotide of embodiment 50, wherein the antisense oligonucleotide or the contiguous nucleotide sequence is a gapmer.
52. The antisense oligonucleotide of embodiment 51, wherein the antisense oligonucleotide or contiguous nucleotide sequence thereof consists of or comprises a gapmer of formula 5′-F-G-F′-3′, where region F and F′ independently comprise or consist of 1-4 2′ sugar modified nucleosides and G is a region between 6 and 18 nucleosides which are capable of recruiting RNaseH.
53. The antisense oligonucleotide of embodiment 52, wherein the 2′ sugar modified nucleoside independently is selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
54. The antisense oligonucleotide of embodiment 52 or 53, wherein one or more of the 2′ sugar modified nucleosides in region F and F′is a LNA nucleoside
55. The antisense oligonucleotide of embodiment 54, wherein all the 2′ sugar modified nucleosides in region F and F′ are LNA nucleosides.
56. The oligonucleotide of embodiment 53 to 55, wherein the LNA nucleoside is selected from beta-D-oxy-LNA, alpha-L-oxy-LNA, beta-D-amino-LNA, alpha-L-amino-LNA, beta-D-thio-LNA, alpha-L-thio-LNA, (S)cET, (R)cET beta-D-ENA and alpha-L-ENA.
57. The antisense oligonucleotide of embodiment 53 to 56, wherein region F and F′ consist of identical LNA nucleosides.
58. The antisense oligonucleotide of embodiment 53 to 57, wherein all the 2′ sugar modified nucleosides in region F and F′ are oxy-LNA nucleosides.
59. The antisense oligonucleotide of any one of embodiments 52 to 58, wherein the nucleosides in region G is DNA and/or alpha-L-LNA nucleosides.
60. The antisense oligonucleotide of embodiment 59, wherein region G consists of at least 75% DNA nucleosides.
61. The antisense oligonucleotide of embodiment 60, where all the nucleosides in region G are DNA nucleosides.
62. The oligonucleotide any one of embodiments 17 to 62, wherein the oligonucleotide is selected from CMP ID NO: 22_1; 23_1; 24_1; 25_1; 26_1; 27_1; 28_1; 29_1; 30_1; 31_1; 32_1; 33_1; 34_1; 35_1; 36_1; 37_1; 38_1; 39_1; 40_1; 41_1; 42_1; 42_2; 42_3; 43_1; 43_2; 44_1; 45_1; 46_1; 49_1; 130_1; 109_1; 83_1; 203_1 and 232_1, or pharmaceutically acceptable salts thereof.
63. A conjugate compound comprising an oligonucleotide according to any one of embodiments 17 to 50 or an antisense oligonucleotide according to any one of embodiments 51 to 62, and at least one conjugate moiety covalently attached to said antisense oligonucleotide.
64. The conjugate compound of embodiment 63, wherein the oligonucleotide is a double stranded siRNA and the conjugate moiety is covalently attached to the sense strand of the siRNA.
65. The conjugate compound of embodiment 63 or 64, wherein the conjugate moiety is selected from carbohydrates, cell surface receptor ligands, drug substances, hormones, lipophilic substances, polymers, proteins, peptides, toxins, vitamins, viral proteins or combinations thereof.
66. The conjugate compound of any one of embodiments 63 to 65, wherein the conjugate moiety is capable of binding to the asialoglycoprotein receptor.
67. The conjugate compound of embodiment 66, wherein the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine.
68. The conjugate compound of embodiment 67, wherein the asialoglycoprotein receptor targeting moiety is N-acetylgalactosamine (GalNAc).
69. The conjugate compound of embodiment 67 or 68, wherein the conjugate moiety is mono-valent, di-valent, tri-valent or tetra-valent with respect to asialoglycoprotein receptor targeting moieties.
70. The conjugate compound of embodiment 69, wherein the conjugate moiety consists of two to four terminal GalNAc moieties and a spacer linking each GalNAc moiety to a brancher molecule that can be conjugated to the antisense compound.
71. The conjugate compound of embodiment 70, wherein the spacer is a PEG spacer.
72. The conjugate compound of embodiment 66 to 71, wherein the conjugate moiety is a tri-valent N-acetylgalactosamine (GalNAc) moiety.
73. The conjugate compound of embodiment 66 to 72, wherein the conjugate moiety is selected from one of the trivalent GalNAc moieties in FIG. 1.
74. The conjugate compound of embodiment 73, wherein the conjugate moiety is the trivalent GalNAc moiety in FIG. 1D.
75. The conjugate compound of embodiment 63-74, comprising a linker which is positioned between the oligonucleotide or the antisense oligonucleotide and the conjugate moiety.
76. The conjugate compound of embodiment 75, wherein the linker is a physiologically labile linker.
77. The conjugate compound of embodiment 76, wherein the physiologically labile linker is nuclease susceptible linker.
78. The oligonucleotide conjugate of embodiment 76 or 77, wherein the physiologically labile linker is composed of 2 to 5 consecutive phosphodiester linkages.
79. The conjugate compound of embodiment 66-78, which display improved cellular distribution between liver vs. kidney or improved cellular uptake into the liver of the conjugate compound as compared to an unconjugated oligonucleotide or antisense oligonucleotide.
80. A pharmaceutical composition comprising a oligonucleotide according to any one of embodiments 17 to 50 or an antisense oligonucleotide according to any one of embodiments 51 to 61, a conjugate compound of embodiment 63 to 79 or acceptable salts thereof and a pharmaceutically acceptable diluent, carrier, salt and/or adjuvant.
81. A method for identifying a compound that prevents, ameliorates and/or inhibits a hepatitis B virus (HBV) infection, comprising:
   a. contacting a test compound with
      i. a RTEL1 polypeptide; or
      ii. a cell expressing RTEL1;
   b. measuring the expression and/or activity of RTEL1 in the presence and absence of said test compound; and
   c. identifying a compound that reduces the expression and/or activity RTEL1 and reduces cccDNA.
82. An in vivo or in vitro method for modulating RTEL1 expression in a target cell which is expressing RTEL1, said method comprising administering the oligonucleotide of any one of embodiments 17 to 50 or an antisense oligonucleotide according to any one of embodiments 51 to 61, a conjugate compound of embodiment 63 to 79 or the pharmaceutical composition of embodiment 80 in an effective amount to said cell.
83. The method of embodiments 82, wherein the RTEL1 expression is reduced by at least 50%, or at least 60%, or at least 70%, or at least 80%, or at least 90%, or at least 95% in the target cell compared to the level without any treatment or treated with a control.
84. The method of embodiments 82, wherein the target cell is infected with HBV and the cccDNA in an HBV infected cell is reduced by at least 50%, or at least 60%, or at least 70%, or at least 80%, or at least 90%, or at least 95% in the HBV infected target cell compared to the level without any treatment or treated with a control.
85. A method for treating or preventing a disease comprising administering a therapeutically or prophylactically effective amount of the oligonucleotide any one of embodiments 17 to 50 or an antisense oligonucleotide according to any one of embodiments 51 to 61, a conjugate compound of embodiment 63 to 79 or the pharmaceutical composition of embodiment 80 to a subject suffering from or susceptible to the disease.
86. The oligonucleotide of any one of embodiments 17 to 50 or the antisense oligonucleotide according to any one of embodiments 51 to 61, or the conjugate compound of any one of embodiments 63 to 79 or the pharmaceutical composition of embodiment 80, for use as a medicament for treatment or prevention of a disease in a subject.
87. Use of the oligonucleotide any one of embodiments 17 to 50 or the antisense oligonucleotide according to any one of embodiments 51 to 61, or the conjugate compound of any one of embodiments 63 to 79 for the preparation of a medicament for treatment or prevention of a disease in a subject.
88. The method, the oligonucleotide, the antisense oligonucleotide, the conjugate or the use of embodiments 85-87 wherein the subject is a mammal.
89. The method, the oligonucleotide, the antisense oligonucleotide, the conjugate, or the use of embodiment 88, wherein the mammal is human.

The invention will now be illustrated by the following examples which have no limiting character.

### EXAMPLES

### Materials and Methods

### Oligonucleotide Motif Sequences and Oligonucleotide Compounds

TABLE 6
list of oligonucleotide motif sequences (indicated by SEQ ID NO),
designs of these, as well as specific oligonucleotide compounds
(indicated by CMP ID NO) designed based on the motif sequence.

| | | | | | Start |
|---|---|---|---|---|---|
| | | | | | position |
| SEQ | | | Oligonucleotide | CMP | on SEQ |
| ID NO | Motif sequence | Design | Compound | ID NO | ID NO: 1 |
| 22 | catggaaggacagtggt | 2-12-3 | CAtggaaggacagtGGT | 22_1 | 8295 |
| 23 | agctttattataacttgaat | 4-14-2 | AGCTttattataacttgaAT | 23_1 | 8684 |
| 24 | cgggacaggtagtaag | 3-10-3 | CGGgacaggtagtAAG | 24_1 | 9668 |
| 25 | cgggacaggtagtaa | 3-9-3 | CGGgacaggtagTAA | 25_1 | 9669 |
| 26 | gcatccaacaagtaattgt | 2-15-2 | GCatccaacaagtaattGT | 26_1 | 9722 |
| 27 | gcatccaacaagtaattg | 2-12-4 | GCatccaacaagtaATTG | 27_1 | 9723 |
| 28 | ggcatccaacaagtaatt | 3-13-2 | GGCatccaacaagtaaTT | 28_1 | 9724 |
| 29 | ggttgggttagaagct | 2-12-2 | GGttgggttagaagCT | 29_1 | 10921 |
| 30 | gcttttacatttaggtttat | 3-15-2 | GCTtttacatttaggtttAT | 30_1 | 11483 |
| 31 | catgttcctttctataact | 3-14-2 | CATgttcctttctataaCT | 31_1 | 11512 |
| 32 | agctttaaattttggtgaa | 3-13-3 | AGCtttaaattttggtGAA | 32_1 | 11622 |
| 33 | ttttacatactctggtcaaa | 2-14-4 | TTttacatactctggtCAAA | 33_1 | 11753 |
| 34 | ttttacatactctggtca | 3-12-3 | TTTtacatactctggTCA | 34_1 | 11755 |
| 35 | gaattttacatactctggtc | 3-14-3 | GAAttttacatactctgGTC | 35_1 | 11756 |
| 36 | gaattttacatactctggt | 2-14-3 | GAattttacatactctGGT | 36_1 | 11757 |
| 37 | gagaattttacatactctgg | 2-15-3 | GAgaattttacatactcTGG | 37_1 | 11758 |
| 38 | atctttgaacacgtctt | 3-11-3 | ATCtttgaacacgtCTT | 38_1 | 12868 |
| 39 | acaaaaaacagtaggtcc | 2-12-4 | ACaaaaaacagtagGTCC | 39_1 | 13234 |
| 40 | ggaataaaacagtaggtc | 2-12-4 | GGaataaaacagtaGGTC | 40_1 | 13551 |
| 41 | agcttcgtcaaagatcac | 3-13-2 | AGCttcgtcaaagatcAC | 41_1 | 14786 |
| 42 | ggtgggtggatgtttc | 1-12-3 | GgtgggtggatgtTTC | 42_1 | 18085 |
| 42 | ggtgggtggatgtttc | 1-11-4 | GgtgggtggatgTTTC | 42_2 | 18085 |
| 42 | ggtgggtggatgtttc | 1-13-2 | GgtgggtggatgttTC | 42_3 | 18085 |
| 43 | ggtggtgtggagaagc | 1-12-3 | GgtggtgtggagaAGC | 43_1 | 22425 |
| 43 | ggtggtgtggagaagc | 1-13-2 | GgtggtgtggagaaGC | 43_2 | 22425 |
| 44 | gctcatactccacacac | 2-13-2 | GCtcatactccacacAC | 44_1 | 33030 |
| 45 | catcggaacccttgtagtcc | 2-16-2 | CAtcggaacccttgtagtCC | 45_1 | 35103 |
| 46 | gatacagacctcctcaaac | 2-15-2 | GAtacagacctcctcaaAC | 46_1 | 35371 |
| 47 | ggtggaggtggtgctgc | 1-14-2 | GgtggaggtggtgctGC | 47_1 | 35636 |
| 48 | aggtggaggtggtgct | 1-12-3 | AggtggaggtggtGCT | 48_1 | 35638 |
| 49 | tggtgtgggagtagca | 2-12-2 | TGgtgtgggagtagCA | 49_1 | 36915 |
| 50 | cgatggcgagaaatta | 4-10-2 | CGATggegagaaatTA | 50_1 | 3824 |
| 51 | taattcagcaaaaaagccca | 3-15-2 | TAAttcagcaaaaaagccCA | 51_1 | 3858 |
| 52 | aagaatctgacacccca | 2-12-3 | AAgaatctgacaccCCA | 52_1 | 3924 |
| 53 | agacagccaagaatctgacac | 1-18-2 | AgacagccaagaatctgacAC | 53_1 | 3928 |
| 54 | cagccaagaatctgaca | 2-12-3 | CAgccaagaatctgACA | 54_1 | 3929 |
| 55 | acaggaacccgacag | 2-10-3 | ACaggaaccegaCAG | 55_1 | 4496 |
| 56 | gttactctcttgtttcttcac | 1-18-2 | GttactctcttgtttcttcAC | 56_1 | 4789 |
| 57 | ttactctcttgtttcttca | 1-16-2 | TtactctcttgtttcttCA | 57_1 | 4790 |
| 57 | ttactctcttgtttcttca | 1-14-4 | TtactctcttgtttcTTCA | 57_2 | 4790 |
| 58 | gttactctcttgtttcttc | 2-15-2 | GTtactctcttgtttctTC | 58_1 | 4791 |
| 59 | cgtgggtggagaagca | 1-13-2 | CgtgggtggagaagCA | 59_1 | 5717 |
| 60 | acgtgggtggagaagc | 2-12-2 | ACgtgggtggagaaGC | 60_1 | 5718 |
| 61 | cagaaactgtaagggca | 1-13-3 | CagaaactgtaaggGCA | 61_1 | 5815 |
| 62 | agggatagcagggaagg | 2-13-2 | AGggatagcagggaaGG | 62_1 | 7246 |
| 63 | gcttaaacacagacaga | 2-11-4 | GCttaaacacagaCAGA | 63_1 | 7501 |
| 64 | tgcttaaacacagacag | 3-11-3 | TGCttaaacacagaCAG | 64_1 | 7502 |
| 65 | cagggcagggaagaacag | 1-14-3 | CagggcagggaagaaCAG | 65_1 | 7845 |
| 66 | catggaaggacagtgg | 3-10-3 | CATggaaggacagTGG | 66_1 | 8296 |
| 66 | catggaaggacagtgg | 1-12-3 | CatggaaggacagTGG | 66_2 | 8296 |
| 67 | ccccctcaatataagaa | 3-12-2 | CCCcctcaatataagAA | 67_1 | 8705 |
| 68 | aaccaaccctattcctgg | 2-14-2 | AAccaaccctattcctGG | 68_1 | 9375 |
| 68 | aaccaaccctattcctgg | 1-15-2 | AaccaaccctattcctGG | 68_2 | 9375 |
| 69 | accaaccctattcctg | 1-12-3 | AccaaccctattcCTG | 69_1 | 9376 |
| 70 | aaccaaccctattcctg | 3-12-2 | AACcaaccctattccTG | 70_1 | 9376 |
| 71 | aaaaccaaccctattcct | 3-12-3 | AAAaccaaccctattCCT | 71_1 | 9377 |
| 72 | aaaccaaccctattcc | 4-10-2 | AAACcaaccctattCC | 72_1 | 9378 |
| 73 | ggtagtaagggcacacc | 1-14-2 | GgtagtaagggcacaCC | 73_1 | 9660 |
| 74 | gacaggtagtaagggcacac | 1-17-2 | GacaggtagtaagggcacAC | 74_1 | 9661 |
| 75 | gtagtaagggcacac | 3-9-3 | GTAgtaagggcaCAC | 75_1 | 9661 |
| 76 | gacaggtagtaagggcaca | 1-16-2 | GacaggtagtaagggcaCA | 76_1 | 9662 |
| 77 | acaggtagtaagggcaca | 2-14-2 | ACaggtagtaagggcaCA | 77_1 | 9662 |
| 78 | gacaggtagtaagggca | 2-13-2 | GAcaggtagtaagggCA | 78_1 | 9664 |
| 79 | cgggacaggtagtaaggg | 1-15-2 | CgggacaggtagtaagGG | 79_1 | 9666 |
| 80 | catccaacaagtaattgt | 3-12-3 | CATccaacaagtaatTGT | 80_1 | 9722 |
| 80 | catccaacaagtaattgt | 2-13-3 | CAtccaacaagtaatTGT | 80_2 | 9722 |
| 81 | ggcatccaacaagtaattgt | 1-16-3 | GgcatccaacaagtaatTGT | 81_1 | 9722 |
| 82 | ggcatccaacaagtaattg | 3-14-2 | GGCatccaacaagtaatTG | 82_1 | 9723 |
| 82 | ggcatccaacaagtaattg | 1-15-3 | GgcatccaacaagtaaTTG | 82_2 | 9723 |
| 83 | ggcatccaacaagtaat | 4-11-2 | GGCAtccaacaagtaAT | 83_1 | 9725 |
| 83 | ggcatccaacaagtaat | 3-12-2 | GGCatccaacaagtaAT | 83_2 | 9725 |
| 84 | cgtgaaggagagaacct | 2-12-3 | CGtgaaggagagaaCCT | 84_1 | 10036 |
| 85 | acgtgaaggagagaacc | 3-12-2 | ACGtgaaggagagaaCC | 85_1 | 10037 |
| 86 | gacgtgaaggagagaacc | 2-13-3 | GAegtgaaggagagaACC | 86_1 | 10037 |
| 86 | gacgtgaaggagagaacc | 2-14-2 | GAegtgaaggagagaaCC | 86_2 | 10037 |
| 85 | acgtgaaggagagaacc | 4-11-2 | ACGTgaaggagagaaCC | 85_2 | 10037 |
| 87 | gacgtgaaggagagaac | 2-11-4 | GAcgtgaaggagaGAAC | 87_1 | 10038 |
| 88 | cagtcttgctatgcct | 2-12-2 | CAgtcttgctatgcCT | 88_1 | 10563 |
| 89 | ctagaatcaaagctcca | 2-12-3 | CTagaatcaaagctCCA | 89_1 | 10591 |
| 90 | acatcgcacttgggc | 1-12-2 | AcategcacttggGC | 90_1 | 10705 |
| 91 | cacggcaaacctcacc | 1-12-3 | CaeggcaaacctcACC | 91_1 | 10851 |
| 92 | aaccacggcaaacctcac | 3-13-2 | AACcaeggcaaacctcAC | 92_1 | 10852 |
| 93 | caaagcaccgagtcacc | 1-13-3 | CaaagcacegagtcACC | 93_1 | 10873 |
| 94 | tcaaagcaccgagtcac | 1-13-3 | TcaaagcacegagtCAC | 94_1 | 10874 |
| 95 | ctggttgggttagaag | 2-10-4 | CTggttgggttaGAAG | 95_1 | 10923 |
| 95 | ctggttgggttagaag | 2-12-2 | CTggttgggttagaAG | 95_2 | 10923 |
| 96 | tataacttttagtttagc | 2-12-4 | TAtaacttttagttTAGC | 96_1 | 11501 |
| 97 | ttcctttctataactttt | 4-12-2 | TTCCtttctataacttTT | 97_1 | 11509 |
| 98 | gttcctttctataactttt | 4-13-2 | GTTCctttctataacttTT | 98_1 | 11509 |
| 99 | gttcctttctataacttt | 4-12-2 | GTTCctttctataactTT | 99_1 | 11510 |
| 100 | atgttcctttctataacttt | 2-15-3 | ATgttcctttctataacTTT | 100_1 | 11510 |
| 101 | atgttcctttctataactt | 2-14-3 | ATgttcctttctataaCTT | 101_1 | 11511 |
| 102 | atgttcctttctataact | 2-14-2 | ATgttcctttctataaCT | 102_1 | 11512 |
| 103 | gctttaatctgccttc | 1-11-4 | GctttaatctgcCTTC | 103_1 | 12697 |
| 104 | ccgtggctttaatctgc | 1-14-2 | CegtggctttaatctGC | 104_1 | 12701 |
| 105 | ccgtggctttaatctg | 2-12-2 | CCgtggctttaatcTG | 105_1 | 12702 |
| 105 | ccgtggctttaatctg | 3-11-2 | CCGtggctttaatcTG | 105_2 | 12702 |
| 106 | caaaaaacagtaggtcc | 2-11-4 | CAaaaaacagtagGTCC | 106_1 | 13234 |
| 106 | caaaaaacagtaggtcc | 3-11-3 | CAAaaaacagtaggTCC | 106_2 | 13234 |
| 107 | gaataaaacagtaggtcc | 2-12-4 | GAataaaacagtagGTCC | 107_1 | 13550 |
| 108 | ggaataaaacagtaggtcc | 4-13-2 | GGAAtaaaacagtaggtCC | 108_1 | 13550 |
| 108 | ggaataaaacagtaggtcc | 2-15-2 | GGaataaaacagtaggtCC | 108_2 | 13550 |
| 108 | ggaataaaacagtaggtcc | 1-14-4 | GgaataaaacagtagGTCC | 108_3 | 13550 |
| 109 | ggaataaaacagtaggt | 3-11-3 | GGAataaaacagtaGGT | 109_1 | 13552 |
| 109 | ggaataaaacagtaggt | 2-11-4 | GGaataaaacagtAGGT | 109_2 | 13552 |
| 110 | ggaataaaacagtagg | 2-10-4 | GGaataaaacagTAGG | 110_1 | 13553 |
| 111 | cacagagtgtcatggg | 1-13-2 | CacagagtgtcatgGG | 111_1 | 14032 |
| 112 | acagcatggaaaggcacg | 1-13-4 | AcagcatggaaaggCACG | 112_1 | 14523 |
| 113 | cagcatggaaaggcacg | 1-12-4 | CagcatggaaaggCACG | 113_1 | 14523 |
| 114 | tacaggaggaagagaagggac | 1-18-2 | TacaggaggaagagaagggAC | 114_1 | 14725 |
| 115 | acaggaggaagagaaggg | 1-13-4 | AcaggaggaagagaAGGG | 115_1 | 14727 |
| 116 | tctacaggaggaagagaa | 4-12-2 | TCTAcaggaggaagagAA | 116_1 | 14730 |
| 116 | tctacaggaggaagagaa | 1-13-4 | TctacaggaggaagAGAA | 116_2 | 14730 |
| 117 | tctacaggaggaagaga | 4-11-2 | TCTAcaggaggaagaGA | 117_1 | 14731 |
| 117 | tctacaggaggaagaga | 2-12-3 | TCtacaggaggaagAGA | 117_2 | 14731 |
| 117 | tctacaggaggaagaga | 2-11-4 | TCtacaggaggaaGAGA | 117_3 | 14731 |
| 118 | cttcgtcaaagatcacg | 2-11-4 | CTtcgtcaaagatCACG | 118_1 | 14785 |
| 119 | gcttcgtcaaagatcacg | 2-13-3 | GCttegtcaaagatcACG | 119_1 | 14785 |
| 120 | gcttcgtcaaagatcac | 3-11-3 | GCTtegtcaaagatCAC | 120_1 | 14786 |
| 120 | gcttcgtcaaagatcac | 2-13-2 | GCttegtcaaagatcAC | 120_2 | 14786 |
| 121 | ccagaaaggtttgcg | 3-10-2 | CCAgaaaggtttgCG | 121_1 | 14874 |
| 122 | tccagaaaggtttgcg | 3-11-2 | TCCagaaaggtttgCG | 122_1 | 14874 |
| 122 | tccagaaaggtttgcg | 1-12-3 | TccagaaaggtttGCG | 122_2 | 14874 |
| 123 | cagaggcatcggatcag | 2-13-2 | CAgaggcateggatcAG | 123_1 | 14974 |
| 124 | cagaggcatcggatca | 3-11-2 | CAGaggcateggatCA | 124_1 | 14975 |
| 125 | agcagaggcatcggatc | 2-13-2 | AGcagaggcateggaTC | 125_1 | 14976 |
| 126 | attcttcacacatcttc | 2-11-4 | ATtcttcacacatCTTC | 126_1 | 16133 |
| 127 | ctatgaacgcacctg | 3-9-3 | CTAtgaaegcacCTG | 127_1 | 16282 |
| 128 | ggctatgaacgcacctg | 1-14-2 | GgctatgaaegcaccTG | 128_1 | 16282 |
| 129 | gctgggagaagacatag | 1-12-4 | GctgggagaagacATAG | 129_1 | 16593 |
| 130 | caaaatgcccttacagtga | 4-13-2 | CAAAatgcccttacagtGA | 130_1 | 16919 |
| 131 | caaaatgcccttacagt | 2-12-3 | CAaaatgcccttacAGT | 131_1 | 16921 |
| 132 | tgtgcgattttaaaggaaaat | 3-15-3 | TGTgegattttaaaggaaAAT | 132_1 | 17525 |
| 133 | catgtgcgattttaaaggaaa | 3-15-3 | CATgtgegattttaaaggAAA | 133_1 | 17527 |
| 134 | tgtgcgattttaaaggaa | 4-12-2 | TGTGegattttaaaggAA | 134_1 | 17528 |
| 135 | catgtgcgattttaaagga | 1-15-3 | CatgtgegattttaaaGGA | 135_1 | 17529 |
| 136 | atgtgcgattttaaagga | 3-13-2 | ATGtgegattttaaagGA | 136_1 | 17529 |
| 137 | accctgtcacttaaatatatg | 1-18-2 | AccctgtcacttaaatataTG | 137_1 | 17712 |
| 138 | gagggaggtggagcgtt | 1-14-2 | GagggaggtggagegTT | 138_1 | 17924 |
| 139 | ctgaagagtggagaagg | 2-11-4 | CTgaagagtggagAAGG | 139_1 | 18130 |
| 139 | ctgaagagtggagaagg | 1-13-3 | CtgaagagtggagaAGG | 139_2 | 18130 |
| 140 | caataaataaagtgtgagga | 3-14-3 | CAAtaaataaagtgtgaGGA | 140_1 | 18454 |
| 141 | caacccagtaaccatgac | 3-13-2 | CAAcccagtaaccatgAC | 141_1 | 19424 |
| 142 | caacccagtaaccatga | 3-12-2 | CAAcccagtaaccatGA | 142_1 | 19425 |
| 143 | accaacccagtaaccatga | 1-16-2 | AccaacccagtaaccatGA | 143_1 | 19425 |
| 144 | gagcaggtgttttatc | 3-11-2 | GAGcaggtgttttaTC | 144_1 | 19825 |
| 145 | ggtcgaggaggtgtcac | 1-14-2 | GgtcgaggaggtgtcAC | 145_1 | 20437 |
| 145 | ggtcgaggaggtgtcac | 2-13-2 | GGtcgaggaggtgtcAC | 145_2 | 20437 |
| 146 | gtcgaggaggtgtcac | 1-11-4 | GtcgaggaggtgTCAC | 146_1 | 20437 |
| 147 | ggtcgaggaggtgtca | 1-13-2 | GgtcgaggaggtgtCA | 147_1 | 20438 |
| 147 | ggtcgaggaggtgtca | 1-12-3 | GgtcgaggaggtgTCA | 147_2 | 20438 |
| 148 | ggtcgaggaggtgtc | 2-11-2 | GGtcgaggaggtgTC | 148_1 | 20439 |
| 149 | ccaggtctcaaaaaggg | 1-13-3 | CcaggtctcaaaaaGGG | 149_1 | 20653 |
| 150 | attacgctgaggaca | 1-10-4 | AttaegctgagGACA | 150_1 | 21489 |
| 151 | cattacgctgaggac | 4-9-2 | CATTaegctgaggAC | 151_1 | 21490 |
| 152 | cttgagcattacgc | 3-8-3 | CTTgagcattaCGC | 152_1 | 21497 |
| 153 | cgaggagaagaaggcag | 3-12-2 | CGAggagaagaaggcAG | 153_1 | 22019 |
| 153 | cgaggagaagaaggcag | 2-11-4 | CGaggagaagaagGCAG | 153_2 | 22019 |
| 154 | ccttggtctgaaacgtgat | 1-15-3 | CcttggtctgaaaegtGAT | 154_1 | 22071 |
| 155 | ctaacgcctccacgc | 1-12-2 | CtaaegcctccacGC | 155_1 | 22281 |
| 156 | ggacaggctctacgg | 1-11-3 | GgacaggctctaCGG | 156_1 | 22312 |
| 157 | actaatacagcaggagaagg | 2-16-2 | ACtaatacagcaggagaaGG | 157_1 | 22964 |
| 158 | aactaatacagcaggagaagg | 1-16-4 | AactaatacagcaggagAAGG | 158_1 | 22964 |
| 158 | aactaatacagcaggagaagg | 1-17-3 | AactaatacagcaggagaAGG | 158_2 | 22964 |
| 159 | taactaatacagcaggagaag | 1-16-4 | TaactaatacagcaggaGAAG | 159_1 | 22965 |
| 160 | ttgaagagccaaccac | 1-11-4 | TtgaagagccaaCCAC | 160_1 | 24131 |
| 161 | ccattttcactgtcaag | 3-12-2 | CCAttttcactgtcaAG | 161_1 | 25605 |
| 162 | gccattttcactgtcaa | 2-12-3 | GCcattttcactgtCAA | 162_1 | 25606 |
| 163 | agaaatgcggagaagc | 2-10-4 | AGaaatgeggagAAGC | 163_1 | 25796 |
| 164 | aaatggaaaaaatgaccagc | 2-14-4 | AAatggaaaaaatgacCAGC | 164_1 | 26188 |
| 165 | aggacttacgacaaaaccac | 1-15-4 | AggacttaegacaaaaCCAC | 165_1 | 26505 |
| 166 | ggacttacgacaaaacca | 2-13-3 | GGacttaegacaaaaCCA | 166_1 | 26506 |
| 167 | gacttacgacaaaacca | 3-11-3 | GACttaegacaaaaCCA | 167_1 | 26506 |
| 168 | acaccaggacttacgaca | 1-14-3 | AcaccaggacttaegACA | 168_1 | 26512 |
| 169 | tagaaattcaacatggc | 1-12-4 | TagaaattcaacaTGGC | 169_1 | 27376 |
| 169 | tagaaattcaacatggc | 4-11-2 | TAGAaattcaacatgGC | 169_2 | 27376 |
| 169 | tagaaattcaacatggc | 2-12-3 | TAgaaattcaacatGGC | 169_3 | 27376 |
| 170 | ctagaaattcaacatggc | 2-13-3 | CTagaaattcaacatGGC | 170_1 | 27376 |
| 171 | gtcatcggttcacc | 1-9-4 | GtcateggttCACC | 171_1 | 27602 |
| 172 | actcgaagacgcca | 2-8-4 | ACtegaagacGCCA | 172_1 | 28539 |
| 173 | gactcgaagacgcc | 3-9-2 | GACtegaagaegCC | 173_1 | 28540 |
| 174 | ggcacaagcagaacgac | 2-13-2 | GGcacaagcagaaegAC | 174_1 | 29235 |
| 175 | agtcagaacaaaggaggc | 1-15-2 | AgtcagaacaaaggagGC | 175_1 | 29668 |
| 176 | gaagtcagaacaaaggag | 4-12-2 | GAAGtcagaacaaaggAG | 176_1 | 29670 |
| 177 | gcagaagtcagaacaaagg | 1-14-4 | GcagaagtcagaacaAAGG | 177_1 | 29672 |
| 178 | gtgcagaagtcagaacaaa | 3-13-3 | GTGcagaagtcagaacAAA | 178_1 | 29674 |
| 178 | gtgcagaagtcagaacaaa | 3-14-2 | GTGcagaagtcagaacaAA | 178_2 | 29674 |
| 179 | gtgcagaagtcagaacaa | 3-13-2 | GTGcagaagtcagaacAA | 179_1 | 29675 |
| 180 | aaggatgagggagcggac | 1-14-3 | AaggatgagggagegGAC | 180_1 | 29894 |
| 181 | gtaaggatgagggagc | 2-12-2 | GTaaggatgagggaGC | 181_1 | 29898 |
| 182 | tggtaaggatgagggag | 1-12-4 | TggtaaggatgagGGAG | 182_1 | 29899 |
| 183 | cgtacatctgcatctc | 2-10-4 | CGtacatctgcaTCTC | 183_1 | 29951 |
| 184 | tgtaagataagaggcaacact | 1-18-2 | TgtaagataagaggcaacaCT | 184_1 | 30947 |
| 185 | ttgtaagataagaggcaacac | 1-17-3 | TtgtaagataagaggcaaCAC | 185_1 | 30948 |
| 186 | ttgtaagataagaggcaaca | 2-14-4 | TTgtaagataagaggcAACA | 186_1 | 30949 |
| 187 | tttgtaagataagaggcaaca | 2-17-2 | TTtgtaagataagaggcaaCA | 187_1 | 30949 |
| 188 | tgtaagataagaggcaa | 2-11-4 | TGtaagataagagGCAA | 188_1 | 30951 |
| 189 | ctggaaggaaagttggt | 2-12-3 | CTggaaggaaagttGGT | 189_1 | 31229 |
| 190 | atagtaagcactgatggtc | 3-14-2 | ATAgtaagcactgatggTC | 190_1 | 31245 |
| 190 | atagtaagcactgatggtc | 1-14-4 | AtagtaagcactgatGGTC | 190_2 | 31245 |
| 191 | tagtaagcactgatgg | 2-11-3 | TAgtaagcactgaTGG | 191_1 | 31247 |
| 192 | catagtaagcactgatg | 3-12-2 | CATagtaagcactgaTG | 192_1 | 31248 |
| 192 | catagtaagcactgatg | 2-11-4 | CAtagtaagcactGATG | 192_2 | 31248 |
| 193 | ctgtaactcacctggc | 1-13-2 | CtgtaactcacctgGC | 193_1 | 31835 |
| 193 | ctgtaactcacctggc | 2-12-2 | CTgtaactcacctgGC | 193_2 | 31835 |
| 194 | cggatcactcgcccg | 1-12-2 | CggatcactegccCG | 194_1 | 32000 |
| 195 | acacaggctactctcgg | 1-14-2 | AcacaggctactcteGG | 195_1 | 33017 |
| 196 | acacaggctactctcg | 3-10-3 | ACAcaggctactcTCG | 196_1 | 33018 |
| 197 | ccacacacaggctactc | 1-14-2 | CcacacacaggctacTC | 197_1 | 33021 |
| 198 | atactccacacacaggct | 1-15-2 | AtactccacacacaggCT | 198_1 | 33025 |
| 199 | atactccacacacaggc | 1-14-2 | AtactccacacacagGC | 199_1 | 33026 |
| 200 | gctcatactccacacacag | 1-16-2 | GctcatactccacacacAG | 200_1 | 33028 |
| 201 | tcatactccacacacag | 2-11-4 | TCatactccacacACAG | 201_1 | 33028 |
| 201 | tcatactccacacacag | 2-13-2 | TCatactccacacacAG | 201_2 | 33028 |
| 202 | gctcatactccacacaca | 1-14-3 | GctcatactccacacACA | 202_1 | 33029 |
| 202 | gctcatactccacacaca | 1-15-2 | GctcatactccacacaCA | 202_2 | 33029 |
| 203 | tgctcatactccacacac | 1-14-3 | TgctcatactccacaCAC | 203_1 | 33030 |
| 203 | tgctcatactccacacac | 1-15-2 | TgctcatactccacacAC | 203_2 | 33030 |
| 203 | tgctcatactccacacac | 2-14-2 | TGctcatactccacacAC | 203_3 | 33030 |
| 204 | agcaggaagcagggagaaa | 2-15-2 | AGcaggaagcagggagaAA | 204_1 | 33562 |
| 205 | tccgaccacagcgag | 2-11-2 | TCegaccacagegAG | 205_1 | 33681 |
| 206 | cagaagccaagggacatg | 1-14-3 | CagaagccaagggacATG | 206_1 | 34432 |
| 206 | cagaagccaagggacatg | 2-14-2 | CAgaagccaagggacaTG | 206_2 | 34432 |
| 207 | cagaagccaagggacat | 2-12-3 | CAgaagccaagggaCAT | 207_1 | 34433 |
| 208 | ccagaccaacacggaaacg | 1-14-4 | CcagaccaacaeggaAACG | 208_1 | 34571 |
| 209 | ccagaccaacacggaaac | 2-12-4 | CCagaccaacaeggAAAC | 209_1 | 34572 |
| 210 | gaatgggcaaagggtaga | 4-12-2 | GAATgggcaaagggtaGA | 210_1 | 34742 |
| 211 | aatgggcaaagggtaga | 2-12-3 | AAtgggcaaagggtAGA | 211_1 | 34742 |
| 210 | gaatgggcaaagggtaga | 2-14-2 | GAatgggcaaagggtaGA | 210_2 | 34742 |
| 212 | gaatgggcaaagggtag | 2-12-3 | GAatgggcaaagggTAG | 212_1 | 34743 |
| 213 | gaacccttgtagtcctg | 1-14-2 | GaacccttgtagtccTG | 213_1 | 35101 |
| 214 | aacccttgtagtcct | 4-9-2 | AACCcttgtagtcCT | 214_1 | 35102 |
| 215 | ggaacccttgtagtc | 2-11-2 | GGaacccttgtagTC | 215_1 | 35104 |
| 216 | atcggaacccttgtagtc | 2-14-2 | ATeggaacccttgtagTC | 216_1 | 35104 |
| 216 | atcggaacccttgtagtc | 1-15-2 | AteggaacccttgtagTC | 216_2 | 35104 |
| 217 | catcggaacccttgtagtc | 1-16-2 | CateggaacccttgtagTC | 217_1 | 35104 |
| 218 | catcggaacccttgtagt | 2-14-2 | CAtcggaacccttgtaGT | 218_1 | 35105 |
| 219 | gatacagacctcctcaaact | 1-17-2 | GatacagacctcctcaaaCT | 219_1 | 35370 |
| 220 | gatacagacctcctcaaac | 2-14-3 | GAtacagacctcctcaAAC | 220_1 | 35371 |
| 221 | gatacagacctcctcaaa | 2-13-3 | GAtacagacctcctcAAA | 221_1 | 35372 |
| 221 | gatacagacctcctcaaa | 2-12-4 | GAtacagacctcctCAAA | 221_2 | 35372 |
| 221 | gatacagacctcctcaaa | 1-13-4 | GatacagacctcctCAAA | 221_3 | 35372 |
| 222 | gatacagacctcctcaa | 2-11-4 | GAtacagacctccTCAA | 222_1 | 35373 |
| 222 | gatacagacctcctcaa | 1-13-3 | GatacagacctcctCAA | 222_2 | 35373 |
| 223 | gccccatttaccagtg | 1-13-2 | GccccatttaccagTG | 223_1 | 35470 |
| 224 | cccaacaagtgatgct | 2-12-2 | CCcaacaagtgatgCT | 224_1 | 35965 |
| 225 | cccaacaagtgatgc | 2-11-2 | CCcaacaagtgatGC | 225_1 | 35966 |
| 226 | gtaccaagcccagaagg | 1-14-2 | GtaccaagcccagaaGG | 226_1 | 36279 |
| 227 | gtaccaagcccagaag | 1-11-4 | GtaccaagcccaGAAG | 227_1 | 36280 |
| 228 | ttcctgatgaagagatg | 4-11-2 | TTCCtgatgaagagaTG | 228_1 | 36549 |
| 229 | tcctgatgaagagatg | 2-10-4 | TCctgatgaagaGATG | 229_1 | 36549 |
| 229 | tcctgatgaagagatg | 3-11-2 | TCCtgatgaagagaTG | 229_2 | 36549 |
| 230 | tgggagtagcatggc | 2-11-2 | TGggagtagcatgGC | 230_1 | 36911 |
| 231 | tgtgggagtagcatggc | 1-14-2 | TgtgggagtagcatgGC | 231_1 | 36911 |
| 232 | gtgggagtagcatggc | 1-13-2 | GtgggagtagcatgGC | 232_1 | 36911 |
| 230 | tgggagtagcatggc | 1-11-3 | TgggagtagcatGGC | 230_2 | 36911 |
| 233 | aaacatgctgaaccctg | 2-11-4 | AAacatgctgaacCCTG | 233_1 | 37254 |
| 234 | acaaacatgctgaaccct | 2-13-3 | ACaaacatgctgaacCCT | 234_1 | 37255 |
| 234 | acaaacatgctgaaccct | 1-14-3 | AcaaacatgctgaacCCT | 234_2 | 37255 |
| 234 | acaaacatgctgaaccct | 3-13-2 | ACAaacatgctgaaccCT | 234_3 | 37255 |
| 235 | cacaaacatgctgaaccc | 2-14-2 | CAcaaacatgctgaacCC | 235_1 | 37256 |
| 236 | cacaaacatgctgaacc | 2-12-3 | CAcaaacatgctgaACC | 236_1 | 37257 |
| 237 | tggacgcacaaacatgc | 1-12-4 | TggaegcacaaacATGC | 237_1 | 37263 |

Motif sequences represent the contiguous sequence of nucleobases present in the oligonucleotide.

Designs refer to the gapmer design, F-G-F′, where each number represents the number of consecutive modified nucleosides, e.g 2′ modified nucleosides (first number=5′ flank), followed by the number of DNA nucleosides (second number=gap region), followed by the number of modified nucleosides, e.g 2′ modified nucleosides (third number=3′ flank), optionally preceded by or followed by further repeated regions of DNA and LNA, which are not necessarily part of the contiguous sequence that is complementary to the target nucleic acid.

Oligonucleotide compounds represent specific designs of a motif sequence. Capital letters represent beta-D-oxy LNA nucleosides, lowercase letters represent DNA nucleosides, all LNA C are 5-methyl cytosine and 5-methyl DNA cytosines are presented by “e”, and all internucleoside linkages are phosphorothioate internucleoside linkages.

### Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

### Elongation of the Oligonucleotide

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC

The crude compounds are purified by preparative RP-HPLC on a Phenomenex Jupiter C18 10 μ150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

### DCI: 4,5-Dicyanoimidazole

### DCM: Dichloromethane

### DMF: Dimethylformamide

### DMT: 4,4′-Dimethoxytrityl

### THF: Tetrahydrofurane

### Bz: Benzoyl

### Ibu: Isobutyryl

RP-HPLC: Reverse phase high performance liquid chromatography

### Tₘ Assay

Oligonucleotide and RNA target (phosphate linked, PO) duplexes are diluted to 3 mM in 500 ml RNase-free water and mixed with 500 ml 2× Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

clonal growth medium (dHCGM). dHCGM is a DMEM medium containing 100 U/ml Penicillin, 100 μg/ml Streptomycin, 20 mM Hepes, 44 mM NaHC03, 15 μg/ml L-proline, 0.25 μg/ml insulin, 50 nM Dexamethazone, 5 ng/ml EGF, 0.1 mM Asc-2P, 2% DMSO and 10% FBS (Ishida et al., 2015). Cells were cultured at 37° C. incubator in a humidified atmosphere with 5% CO2. Culture medium was replaced 24 h post-plating and every 2 days until harvest.

### Primary Human Hepatocytes (PXB-PHH)

Fresh primary human hepatocytes (PXB-PHH) harvested from humanized mice (uPA/SCID mice)—herein called PHH—were obtained from PhoenixBio Co., Ltd (Japan). Cells were seeded on a collagen I-coated plate at the following cell density: 35,000 cells/well (384-well), 70,000 cells/well (96-well), or, 400,000 cells/well (24-well) in modified hepatocyte clonal growth medium (dHCGM). dHCGM is a DMEM medium containing 100 U/ml Penicillin, 100 μg/ml Streptomycin, 20 mM Hepes, 44 mM NaHC03, 15 μg/ml L-proline, 0.25 μg/ml insulin, 50 nM Dexamethazone, 5 ng/ml EGF, 0.1 mM Asc-2P, 2% DMSO and 10% FBS (Ishida et al., 2015). Cells were cultured at 37° C. incubator in a humidified atmosphere with 5% CO2. Culture medium was replaced 24 h post-plating and every 2 days until harvest.

### HBV Infection and Oligonucleotide Treatment

PHH were incubated with HBV (purified from CHB individuals) at multiplicity of infection (MOI) of 40 together with 4% PEG for 24 hr; virus inoculum was removed the following day. To allow for cccDNA establishment compound treatment in PHH was started at day 3 post HBV infection. Fresh oligonucleotide dissolved in medium was replenished every 2 days (example 1) or fresh oligonucleotide on day 3, 5, 7 and 9 and after that medium was replenished every 2 days (example 2) until cells were harvested at day 19.

### HBV Antigen Measurements

To evaluate the impact on HBV antigen expression and secretion, supernatants were collected on Day 19. The HBV propagation parameters, HBsAg and HBeAg levels, were measured using CLIA ELISA Kits (Autobio Diagnostic #CL0310-2, #CL0312-2), according to the manufacturer's protocol. Briefly, 25 μL of supernatant per well were transferred to the respective antibody coated microtiter plate and 25 μL of enzyme conjugate reagent were added. The plate was incubated for 60 min on a shaker at room temperature before the wells were washed five times with washing buffer using an automatic washer. 25 μL of substrate A and B were added to each well. The plates were incubated on a shaker for 10 min at room temperature before luminescence was measured using an Envision luminescence reader (Perkin Elmer).

### CCK8 Cellular Toxicity Measurements

To evaluate the impact of cellular toxicity upon treatment of oligonucleotide, cells were treated as described in HBV infection and oligonucleotide treatment and at Day 18, PHH were pre-incubated for 24 hours at 37° C. incubator in a humidified atmosphere with 5% CO₂. 10 ul of CCK-8 solution was added to 100 ul in each well of a 96 well plate and incubated for 1-4 hours. Absorbance at 450 nM using a microplate reader (Tecan) was measured for each plate and values are calculated as % of control (untreated cells).

### Real-Time PCR for Intracellular HBV PgRNA and RTEL1 RNA

mRNA was extracted from the cells using a Qiagen BioRobot Universal System and the RNeasy 96 well Extraction Plates (RNeasy 96 BioRobot 8000 Kit (12)/Cat No./ID: 967152) according to the manufacturer's protocol. The relative HBV and cellular mRNA expression levels were analyzed using Real-time PCR on the ABI QuantStudio 12k Flex.

Beta-actin (ACT B) and HBV pgRNA were quantified by qPCR using TaqMan Fast Advanced Master Mix (Life Technologies, cat no. 4444558) in technical triplicates. Results were normalized over the human ACT B endogenous control. The mRNA expression was analyzed using the comparative cycle threshold 2-ΔΔCt method normalized to the reference gene ACT B and to non-transfected cells. Primers used for ACTB RNA and HBV pgRNA quantification are listed in table 7.

TABLE 7
ACT B and HBV pgRNA qPCR primers

| | | | Seq |
|---|---|---|---|
| Parameter | Direction | Primer Sequence | ID No |
| HBV pgRNA | Fwd | 5′-GGAGTGTGGAT | 238 |
| | | TCGCACTCCT-3′ | |
| | Rev | 5′-AGATTGAGATC | 239 |
| | | TTCTGCGAC-3′ | |
| | Probe | [6FAM]-AGGCAGG | 240 |
| | | TCCCCTAGAAGAA | |
| | | GAACTCC-[BHQ1] | |
| RTEL1 | Fwd | 5′-CCATCCTGGAC | 241 |
| | | ATTGAGGACT-3′ | |
| | Rev | 5′-CAGGTTCCGGG | 242 |
| | | ACAGGTAGTA-3′ | |

Housekeeping gene primers ACT B (VIC): Hs01060665_g1 (Thermo Fisher Scientific)

### HBV CccDNA Quantification

DNA was extracted from HBV infected Primary Human Hepatocytes using an SDS Lysis Buffer and purified using the ZymoResearch Genomic DNA Clean & Concentrator kit (ZymoResearch, cat no. D4067) protocol. cccDNA levels were determined after digestion with T5 exonuclease (New England Biolabs, MA, USA) using 10 U of T5 for 500 ng of DNA, 1 hour at 37° C. in 20 ul total volume. After digestion, the samples were diluted to 50 ul of which 4 ul were used for the qPCR reaction. The mRNA expression was analyzed using the comparative cycle threshold 2-ΔΔCt method normalized to the reference gene mitochondrial DNA and to non-transfected cells. Quantitative real-time polymerase chain reaction measurements were performed on the QuantStudio 12K Flex PCR System (Applied Biosystems). qPCR was performed with the Fast SYBR™ Green Master Mix (Life Technologies, Cat.No 4385612). Primer are shown in table 8.

TABLE 8
cccDNA qPCR primers.

| | | | Seq |
|---|---|---|---|
| Parameter | Direction | Primer Sequence | ID No |
| cccDNA | Fwd | 5′-CGTCTGTGCCT | 243 |
| | | TCTCATCTGC-3′ | |
| | Rev | 5′-GCACAGCTTGG | 244 |
| | | AGGCTTGAA-3′ | |
| mitochondrial | Fwd | 5′-CCGTCTGAACT | 245 |
| DNA | | ATCCTGCCC-3′ | |
| | Rev | 5′-GCCGTAGTCGG | 246 |
| | | TGTACTCGT-3′ | |

### Example 1: Effect of Antisense Oligonucleotides Targeting RTEL1 on HBV Parameters in HBV Infected PHH

In the following experiment, the effect of RTEL1 knock-down on the HBV parameters, HBsAg, HBeAg, HBV pgRNA and cccDNA, were tested using the oligonucleotide compounds in table 6.

PHH were cultured as described in the Materials and Methods section. The cells were dosed at a final oligonucleotide concentration of 10 μM dissolved in dHCGM Medium at Day 3 post HBV infection with a final culture volume of 100 μl/well. The experiment was performed in biological triplicate and cells were harvested at Day 19 post HBV infection replenishing oligonucleotide every 2 days. According to Materials and Methods, cellular toxicity was determined using CCK8, supernatant was collected to measure HBsAg and HBeAg and cells were harvested in two fractions; one for RTEL1 mRNA and pgRNA measurements using one lysis buffer and one for cccDNA measurements using another lysis buffers as described in Materials and Methods. All values are shown in Table 9 as % of control (untreated cells) i.e. for RTEL1 mRNA, cccDNA and pgRNA the lower the value the larger the inhibition.

CCK8 cellular toxicity was measured as described in the Materials and Methods section to confirm that any reduction in the viral parameters is not the cause of cell death, the closer the value is to 100% the lower the toxicity. The results are shown in table 9.

TABLE 9
RTEL1 mediated cccDNA degradation and inhibition of downstream products

| | % CCK8 | | % RTEL1 | | % cccDNA | | % pgRNA | | % HBsAg | | % HBeAg | |
|---|---|---|---|---|---|---|---|---|---|---|---|---|
| Comp | of Control | | of Control | | of Control | | of Control | | of Control | | of Control | |
| ID NO | Mean | SD | Mean | SD | Mean | SD | Mean | SD | Mean | SD | Mean | SD |
| 22_1 | 93 | 10 | 68 | 12 | 19 | 4 | 55 | 9 | 60 | 48 | 32 | 28 |
| 23_1 | 98 | 7 | 40 | 1 | 46 | 15 | 57 | 18 | 113 | 5 | 140 | 14 |
| 24_1 | 100 | 2 | 9 | 1 | 37 | 13 | 52 | 12 | 106 | 5 | 110 | 24 |
| 25_1 | 99 | 5 | 14 | 0 | 69 | 15 | 57 | 8 | 99 | 16 | 54 | 10 |
| 26_1 | 95 | 4 | 3 | 1 | 38 | 12 | 44 | 3 | 78 | 2 | 74 | 12 |
| 27_1 | 84 | 2 | 4 | 1 | 45 | 16 | 31 | 15 | 106 | 7 | 79 | 8 |
| 28_1 | 92 | 0 | 31 | 14 | 35 | 37 | 7 | 3 | 4 | 0 | 27 | 25 |
| 29_1 | 89 | 7 | 33 | 4 | 24 | 6 | 80 | 15 | 81 | 3 | 54 | 4 |
| 32_1 | 115 | 9 | 25 | 3 | 22 | 3 | 104 | 14 | 100 | 6 | 109 | 14 |
| 35_1 | 97 | 6 | 4 | 1 | 27 | 0 | 115 | 24 | 80 | 5 | 76 | 7 |
| 36_1 | 96 | 7 | 14 | 1 | 43 | 18 | 129 | 35 | 102 | 15 | 91 | 27 |
| 37_1 | 103 | 14 | 8 | 4 | 27 | 11 | 127 | 41 | 91 | 4 | 90 | 9 |
| 38_1 | 107 | 6 | 14 | 3 | 47 | 11 | 129 | 12 | 123 | 5 | 141 | 10 |
| 39_1 | 107 | 13 | 29 | 1 | 26 | 10 | 107 | 61 | 124 | 4 | 112 | 30 |
| 40_1 | 101 | 2 | 48 | 33 | 19 | 5 | 29 | 4 | 94 | 13 | 88 | 19 |
| 41_1 | 112 | 15 | 31 | 9 | 42 | 16 | 129 | 18 | 134 | 31 | 79 | 37 |
| 42_1 | 100 | 1 | 27 | 4 | 17 | 19 | 72 | 13 | 15 | 13 | 22 | 0 |
| 42_2 | 94 | 6 | 23 | 4 | 6 | 4 | 76 | 8 | 30 | 2 | 26 | 1 |
| 42_3 | 100 | 1 | 29 | 5 | 30 | 13 | 78 | 12 | 22 | 5 | 21 | 4 |
| 43_1 | 97 | 1 | 34 | 12 | 8 | 5 | 66 | 3 | 78 | 6 | 49 | 9 |
| 43_2 | 121 | 17 | 48 | 5 | 26 | 4 | 70 | 10 | 95 | 27 | 98 | 36 |
| 46_1 | 124 | 13 | 21 | 4 | 37 | 17 | 130 | 11 | 102 | 3 | 74 | 9 |
| 49_1 | 96 | 9 | 57 | 14 | 36 | 23 | 30 | 10 | 10 | 2 | 15 | 2 |

*RTEL1 and HBV pgRNA is normalized to housekeeping gene.

All the tested antisense oligonucleotides targeting RTEL1 display target knockdown and HBV antiviral efficacy at a single time point of measurement against at least one of the parameters cccDNA, pgRNA, HBsAg and HBeAg with no observed cellular toxicity measured by CCK8.

### Example 2: Additional Oligonucleotide Library Targeting RTEL1 Tested for Effect on CccDNA in Infected PHH

In the following experiment an additional library of 236 oligonucleotides targeting across the RTEL1 transcript was generated, shown in table 6 as CMP ID NO: 50_1 to 237_1. The ability to reduce RTEL1 as well as cccDNA, was tested.

PHH were cultured as described in the Materials and Methods section. The cells were dosed at a final oligonucleotide concentration of 10 μM dissolved in dHCGM Medium at Day 3. 5. 7 and 9 post HBV infection with a final culture volume of 100 μl/well. The experiment was performed in biological triplicate and cells were harvested at Day 19 post HBV infection with replenishing medium every 2 days until day 19.

According to Materials and Methods, cells were harvested in two fractions; one for RTEL1 mRNA using one lysis buffer and one for cccDNA measurements using another lysis buffers as described in Materials and Methods. All values are shown in Table 10 as % of control (untreated cells) i.e. for RTEL1 mRNA and cccDNA the lower the value the larger the inhibition/reduction.

TABLE 10
RTEL1 reduction and cccDNA
degradation following oligonucleotide treatment

| Comp | % RTEL1 of Control | | % cccDNA of Control | |
|---|---|---|---|---|
| ID NO | Mean | SD | Mean | SD |
| 50_1 | 7.01 | 1.47 | 91.93 | 53.33 |
| 51_1 | 23.34 | 5.14 | 91.90 | 100.79 |
| 52_1 | 4.97 | 3.32 | 73.18 | 18.65 |
| 53_1 | 74.82 | 16.01 | 52.34 | 6.08 |
| 54_1 | 23.96 | 11.87 | 84.18 | 48.67 |
| 55_1 | 35.59 | 7.17 | 93.69 | 8.65 |
| 56_1 | 78.85 | 7.76 | 79.38 | 6.37 |
| 57_1 | 43.89 | 3.45 | 56.65 | 21.45 |
| 57_2 | 33.43 | 20.36 | 53.42 | 24.96 |
| 58_1 | 23.10 | 3.28 | 55.61 | 24.65 |
| 59_1 | 84.74 | 22.73 | 83.30 | 8.28 |
| 60_1 | 64.90 | 34.31 | 51.76 | 9.74 |
| 61_1 | 40.04 | 6.27 | 105.58 | 36.39 |
| 62_1 | 48.58 | 7.48 | 69.29 | 5.28 |
| 63_1 | 12.26 | 1.26 | 104.61 | 34.95 |
| 64_1 | 9.23 | 5.14 | 101.03 | 20.54 |
| 65_1 | 44.85 | 17.33 | 101.27 | 9.05 |
| 66_1 | 69.93 | 1.72 | 57.89 | 17.40 |
| 66_2 | 68.17 | 6.74 | 86.07 | 8.18 |
| 67_1 | 34.99 | 34.99 | 56.88 | 31.24 |
| 68_1 | 49.49 | 15.69 | 108.68 | 37.37 |
| 68_2 | 68.22 | 14.89 | 100.98 | 74.61 |
| 69_1 | 86.75 | 3.21 | 96.31 | 6.98 |
| 70_1 | 30.76 | 11.20 | 105.62 | 22.24 |
| 71_1 | 41.40 | 9.03 | 77.41 | 55.10 |
| 72_1 | 42.31 | 3.14 | 84.91 | 30.20 |
| 73_1 | 56.43 | 2.53 | 52.43 | 7.92 |
| 74_1 | 62.53 | 11.46 | 63.20 | 10.15 |
| 75_1 | 27.73 | 0.03 | 54.09 | 8.64 |
| 76_1 | 68.10 | 16.93 | 60.01 | 41.58 |
| 77_1 | 25.25 | 8.17 | 97.07 | 11.51 |
| 78_1 | 24.30 | 4.02 | 57.29 | 3.26 |
| 79_1 | 34.02 | 5.55 | 52.04 | 8.90 |
| 80_1 | 64.76 | 6.15 | 92.09 | 8.09 |
| 80_2 | 100.95 | 0.38 | 91.57 | 16.17 |
| 81_1 | 14.80 | 0.00 | 61.33 | 9.38 |
| 82_1 | 7.11 | 1.75 | 54.70 | 15.77 |
| 82_2 | 9.81 | 1.64 | 58.75 | 1.67 |
| 83_1 | 36.85 | 0.00 | 69.70 | 7.20 |
| 83_2 | 8.21 | 4.88 | 66.53 | 7.60 |
| 84_1 | 62.48 | 11.67 | 93.31 | 8.13 |
| 85_1 | 44.09 | 9.50 | 61.00 | 9.51 |
| 85_2 | 46.72 | 4.15 | 97.36 | 24.50 |
| 86_1 | 80.90 | 31.21 | 51.63 | 23.28 |
| 86_2 | 78.93 | 4.86 | 79.87 | 49.47 |
| 87_1 | 26.46 | 3.80 | 58.61 | 6.02 |
| 88_1 | 19.44 | 0.42 | 80.75 | 14.07 |
| 89_1 | 86.51 | 25.84 | 58.06 | 27.75 |
| 90_1 | 58.49 | 9.86 | 97.93 | 30.79 |
| 91_1 | 90.34 | 8.97 | 70.59 | 11.83 |
| 92_1 | 53.35 | 13.77 | 92.14 | 13.18 |
| 93_1 | 86.17 | 18.80 | 68.78 | 12.94 |
| 94_1 | 82.51 | 34.51 | 74.00 | 6.73 |
| 95_1 | 28.32 | 13.87 | 81.77 | 0.96 |
| 95_2 | 31.64 | 0.23 | 54.23 | 0.02 |
| 96_1 | 65.92 | 43.39 | 90.65 | 44.78 |
| 97_1 | 67.17 | 19.66 | 91.10 | 45.98 |
| 98_1 | 20.59 | 11.95 | 70.09 | 7.77 |
| 99_1 | 45.17 | 11.96 | 81.29 | 17.73 |
| 100_1 | 17.96 | 8.21 | 72.67 | 33.48 |
| 101_1 | 17.68 | 4.56 | 59.25 | 15.63 |
| 102_1 | 21.78 | 7.52 | 56.99 | 3.77 |
| 103_1 | 19.65 | 2.57 | 97.02 | 4.67 |
| 104_1 | 46.82 | 22.92 | 55.25 | 3.33 |
| 105_1 | 27.66 | 3.21 | 72.99 | 3.87 |
| 105_2 | 48.60 | 13.68 | 83.37 | 14.25 |
| 106_1 | 29.66 | 11.05 | 102.77 | 23.24 |
| 106_2 | 56.28 | 1.60 | 50.25 | 12.84 |
| 107_1 | 47.82 | 18.86 | 57.44 | 34.75 |
| 108_1 | 26.06 | 6.57 | 53.90 | 14.97 |
| 108_2 | 9.75 | 2.24 | 59.30 | 0.53 |
| 108_3 | 51.83 | 0.77 | 69.13 | 12.27 |
| 109_1 | 5.91 | 2.61 | 52.52 | 12.19 |
| 109_2 | 6.37 | 1.45 | 68.41 | 17.92 |
| 110_1 | 11.12 | 1.90 | 71.58 | 47.02 |
| 111_1 | 84.19 | 10.48 | 55.32 | 2.17 |
| 112_1 | 41.08 | 12.50 | 52.72 | 6.13 |
| 113_1 | 100.00 | 0.00 | 78.91 | 6.77 |
| 114_1 | 99.08 | 41.68 | 57.06 | 6.19 |
| 115_1 | 25.63 | 22.23 | 72.30 | 60.35 |
| 116_1 | 87.50 | 1.46 | 108.23 | 75.62 |
| 116_2 | 83.18 | 0.17 | 105.88 | 5.43 |
| 117_1 | 76.07 | 4.23 | 67.01 | 6.43 |
| 117_2 | 109.05 | 3.40 | 108.67 | 30.57 |
| 117_3 | 93.69 | 11.88 | 76.35 | 2.30 |
| 118_1 | 46.05 | 10.08 | 72.32 | 8.58 |
| 119_1 | 54.19 | 20.46 | 90.03 | 4.64 |
| 120_1 | 29.75 | 11.69 | 90.50 | 26.06 |
| 120_2 | 47.34 | 0.97 | 101.98 | 12.10 |
| 121_1 | 34.63 | 9.13 | 103.04 | 31.21 |
| 122_1 | 23.84 | 3.45 | 60.00 | 1.08 |
| 122_2 | 49.03 | 3.58 | 57.10 | 15.69 |
| 123_1 | 87.91 | 14.47 | 80.61 | 3.32 |
| 124_1 | 45.34 | 4.29 | 97.91 | 15.74 |
| 125_1 | 65.93 | 8.36 | 82.38 | 1.51 |
| 126_1 | 19.63 | 1.74 | 67.12 | 12.80 |
| 127_1 | 28.07 | 3.10 | 59.52 | 3.75 |
| 128_1 | 46.11 | 9.85 | 89.13 | 11.64 |
| 129_1 | 83.14 | 23.83 | 88.56 | 9.69 |
| 130_1 | 3.01 | 1.73 | 51.18 | 0.78 |
| 131_1 | 5.14 | 1.68 | 106.08 | 38.37 |
| 132_1 | 94.17 | 18.86 | 85.17 | 13.15 |
| 133_1 | 37.12 | 8.11 | 71.11 | 12.56 |
| 134_1 | 19.28 | 5.14 | 68.39 | 8.54 |
| 135_1 | 17.83 | 0.86 | 84.76 | 4.78 |
| 136_1 | 5.76 | 3.37 | 91.47 | 13.69 |
| 137_1 | 91.76 | 19.69 | 89.85 | 32.57 |
| 138_1 | 73.95 | 8.38 | 50.33 | 10.55 |
| 139_1 | 54.46 | 1.17 | 70.90 | 15.24 |
| 139_2 | 52.71 | 12.53 | 52.57 | 17.88 |
| 140_1 | 53.81 | 13.82 | 55.02 | 39.17 |
| 141_1 | 24.70 | 4.90 | 93.03 | 59.35 |
| 142_1 | 19.89 | 3.31 | 54.31 | 2.48 |
| 143_1 | 31.38 | 2.16 | 62.23 | 54.78 |
| 144_1 | 18.80 | 15.78 | 65.31 | 22.34 |
| 145_1 | 42.42 | 4.00 | 65.22 | 31.76 |
| 145_2 | 57.20 | 14.77 | 66.82 | 56.81 |
| 146_1 | 74.65 | 9.52 | 58.95 | 24.13 |
| 147_1 | 109.87 | 31.74 | 71.13 | 40.86 |
| 147_2 | 49.79 | 11.64 | 66.49 | 6.77 |
| 148_1 | 99.53 | 13.20 | 82.39 | 6.25 |
| 149_1 | 63.54 | 1.61 | 58.26 | 12.29 |
| 150_1 | 38.91 | 3.01 | 85.40 | 38.11 |
| 151_1 | 75.08 | 50.61 | 99.88 | 1.35 |
| 152_1 | 36.80 | 3.08 | 53.63 | 9.51 |
| 153_1 | 38.60 | 2.68 | 103.42 | 45.69 |
| 153_2 | 28.05 | 7.82 | 74.47 | 62.74 |
| 154_1 | 41.59 | 7.01 | 77.58 | 12.13 |
| 155_1 | 68.84 | 9.18 | 56.76 | 20.70 |
| 156_1 | 58.21 | 14.18 | 69.70 | 8.08 |
| 157_1 | 70.14 | 24.39 | 58.48 | 4.65 |
| 158_1 | 48.72 | 1.12 | 92.78 | 2.45 |
| 158_2 | 96.47 | 44.09 | 63.51 | 2.33 |
| 159_1 | 69.81 | 9.81 | 63.86 | 5.72 |
| 160_1 | 18.16 | 0.57 | 76.72 | 8.87 |
| 161_1 | 4.70 | 1.81 | 63.24 | 27.23 |
| 162_1 | 1.23 | 0.24 | 96.00 | 1.23 |
| 163_1 | 18.96 | 8.11 | 107.22 | 28.58 |
| 164_1 | 20.71 | 8.98 | 100.94 | 10.67 |
| 165_1 | 54.95 | 11.34 | 79.93 | 2.08 |
| 166_1 | 83.13 | 18.00 | 72.36 | 39.67 |
| 167_1 | 105.82 | 73.31 | 69.39 | 13.42 |
| 168_1 | 56.30 | 26.26 | 61.25 | 5.61 |
| 169_1 | 68.48 | 18.97 | 102.59 | 5.31 |
| 169_2 | 40.76 | 10.27 | 71.29 | 13.34 |
| 169_3 | 12.35 | 7.59 | 103.66 | 4.18 |
| 170_1 | 62.45 | 13.28 | 101.40 | 4.46 |
| 171_1 | 48.84 | 14.22 | 56.66 | 4.08 |
| 172_1 | 65.73 | 10.35 | 86.22 | 58.44 |
| 173_1 | 74.87 | 8.18 | 57.84 | 1.98 |
| 174_1 | 32.77 | 6.46 | 65.95 | 17.39 |
| 175_1 | 14.82 | 5.49 | 77.83 | 11.93 |
| 176_1 | 10.54 | 4.41 | 64.33 | 32.00 |
| 177_1 | 9.41 | 2.42 | 60.66 | 6.42 |
| 178_1 | 2.79 | 1.65 | 76.11 | 8.17 |
| 178_2 | 1.61 | 1.09 | 82.07 | 37.30 |
| 179_1 | 2.40 | 0.97 | 100.84 | 2.49 |
| 180_1 | 28.81 | 9.47 | 64.36 | 9.58 |
| 181_1 | 39.34 | 6.66 | 64.81 | 42.15 |
| 182_1 | 40.14 | 7.85 | 50.35 | 5.83 |
| 183_1 | 27.21 | 11.98 | 96.97 | 14.85 |
| 184_1 | 54.48 | 12.67 | 97.69 | 3.13 |
| 185_1 | 97.74 | 21.37 | 57.01 | 9.95 |
| 186_1 | 13.48 | 2.66 | 76.92 | 17.59 |
| 187_1 | 99.48 | 9.54 | 68.99 | 6.50 |
| 188_1 | 4.89 | 1.53 | 75.04 | 5.16 |
| 189_1 | 62.35 | 14.74 | 72.42 | 7.24 |
| 190_1 | 30.13 | 3.34 | 53.95 | 16.62 |
| 190_2 | 70.42 | 8.41 | 57.21 | 7.44 |
| 191_1 | 13.78 | 2.44 | 59.40 | 8.50 |
| 192_1 | 33.66 | 5.65 | 54.13 | 15.57 |
| 192_2 | 52.23 | 12.88 | 81.54 | 6.25 |
| 193_1 | 72.41 | 7.09 | 79.15 | 22.21 |
| 193_2 | 45.71 | 16.85 | 54.47 | 27.40 |
| 194_1 | 27.32 | 14.47 | 76.00 | 7.91 |
| 195_1 | 44.50 | 2.82 | 51.02 | 9.90 |
| 196_1 | 7.35 | 2.01 | 62.45 | 40.44 |
| 197_1 | 60.12 | 8.74 | 59.04 | 23.63 |
| 198_1 | 17.64 | 9.05 | 72.08 | 10.88 |
| 199_1 | 11.67 | 2.87 | 71.46 | 24.06 |
| 200_1 | 22.21 | 1.07 | 105.37 | 3.83 |
| 201_1 | 2.79 | 2.14 | 67.89 | 3.06 |
| 201_2 | 2.14 | 0.14 | 79.46 | 18.07 |
| 202_1 | 8.36 | 1.66 | 81.72 | 3.96 |
| 202_2 | 17.71 | 3.55 | 91.16 | 6.78 |
| 203_1 | 6.91 | 1.51 | 55.95 | 4.33 |
| 203_2 | 10.41 | 6.90 | 75.76 | 2.97 |
| 203_3 | 41.89 | 0.58 | 61.00 | 6.57 |
| 204_1 | 94.01 | 4.50 | 88.29 | 7.73 |
| 205_1 | 13.51 | 5.81 | 109.03 | 20.51 |
| 206_1 | 89.89 | 7.82 | 85.70 | 3.32 |
| 206_2 | 67.36 | 7.93 | 60.55 | 9.50 |
| 207_1 | 23.15 | 9.64 | 60.44 | 29.16 |
| 208_1 | 92.51 | 27.58 | 75.99 | 10.80 |
| 209_1 | 92.08 | 9.26 | 52.56 | 6.76 |
| 210_1 | 12.27 | 6.71 | 76.30 | 31.02 |
| 210_2 | 89.62 | 18.30 | 77.25 | 3.97 |
| 211_1 | 39.78 | 3.92 | 88.22 | 14.53 |
| 212_1 | 36.19 | 2.39 | 56.65 | 14.73 |
| 213_1 | 26.52 | 14.58 | 62.72 | 7.94 |
| 214_1 | 21.32 | 6.14 | 62.15 | 15.02 |
| 215_1 | 22.10 | 3.45 | 97.49 | 19.57 |
| 216_1 | 29.74 | 1.46 | 53.53 | 20.26 |
| 216_2 | 31.17 | 7.18 | 100.84 | 59.55 |
| 217_1 | 36.16 | 21.08 | 73.76 | 5.05 |
| 218_1 | 26.08 | 3.63 | 62.45 | 6.34 |
| 219_1 | 27.98 | 0.88 | 96.57 | 11.43 |
| 220_1 | 35.67 | 2.59 | 103.11 | 28.19 |
| 221_1 | 21.87 | 3.53 | 80.38 | 6.57 |
| 221_2 | 38.08 | 4.00 | 92.15 | 12.10 |
| 221_3 | 40.64 | 9.61 | 107.45 | 5.84 |
| 222_1 | 19.69 | 3.07 | 59.72 | 7.41 |
| 222_2 | 30.77 | 10.21 | 54.26 | 1.45 |
| 223_1 | 78.85 | 28.60 | 90.08 | 12.56 |
| 224_1 | 26.92 | 15.16 | 71.93 | 15.87 |
| 225_1 | 30.24 | 6.80 | 109.61 | 36.24 |
| 226_1 | 35.09 | 15.62 | 107.11 | 10.01 |
| 227_1 | 37.63 | 12.68 | 70.20 | 8.73 |
| 228_1 | 87.69 | 10.70 | 72.25 | 4.00 |
| 229_1 | 44.99 | 22.32 | 77.03 | 46.39 |
| 229_2 | 63.89 | 35.36 | 51.67 | 29.57 |
| 230_1 | 73.08 | 17.58 | 90.57 | 53.08 |
| 230_2 | 36.46 | 5.34 | 61.99 | 30.47 |
| 231_1 | 45.85 | 28.46 | 67.18 | 23.44 |
| 232_1 | 15.33 | 10.56 | 58.98 | 30.05 |
| 233_1 | 92.94 | 7.62 | 60.18 | 12.23 |
| 234_1 | 36.40 | 26.60 | 60.64 | 20.11 |
| 234_2 | 36.46 | 9.00 | 76.75 | 15.69 |
| 234_3 | 55.70 | 17.41 | 72.99 | 21.80 |
| 235_1 | 85.79 | 19.15 | 69.06 | 8.99 |
| 236_1 | 50.52 | 7.64 | 85.61 | 8.75 |
| 237_1 | 94.23 | 25.38 | 79.07 | 31.59 |

CCK8 cellular toxicity was measured as described in the Materials and Methods section to assess if reduction in the viral parameters could be caused by cell death, the closer the value is to 100% the lower the toxicity. The results are shown in table 11. For CCK8 values above 80% of control it is not considered likely that cell death has an impact on the RTEL1 and cccDNA reduction shown in table 10.

TABLE 11
CCK8 cellular toxicity

| | CCK8 % of Control | |
|---|---|---|
| Comp ID NO | Mean | SD |
| 50_1 | 116.26 | 8.82 |
| 51_1 | 105.05 | 7.05 |
| 52_1 | 82.96 | 6.08 |
| 53_1 | 134.27 | 5.46 |
| 54_1 | 103.38 | 9.14 |
| 55_1 | 71.55 | 5.78 |
| 56_1 | 95.93 | 20.93 |
| 57_1 | 105.94 | 3.64 |
| 57_2 | 111.45 | 11.03 |
| 58_1 | 107.29 | 9.64 |
| 59_1 | 88.76 | 7.99 |
| 60_1 | 107.26 | 3.18 |
| 61_1 | 123.93 | 12.61 |
| 62_1 | 108.72 | 7.62 |
| 63_1 | 98.97 | 11.62 |
| 64_1 | 76.87 | 11.82 |
| 65_1 | 101.36 | 5.18 |
| 66_1 | 89.28 | 6.92 |
| 66_2 | 106.42 | 0.96 |
| 67_1 | 28.82 | 0.83 |
| 68_1 | 118.08 | 11.35 |
| 68_2 | 125.36 | 11.78 |
| 69_1 | 97.66 | 16.30 |
| 70_1 | 120.71 | 12.95 |
| 71_1 | 102.41 | 6.80 |
| 72_1 | 83.17 | 6.40 |
| 73_1 | 55.40 | 2.16 |
| 74_1 | 118.35 | 4.36 |
| 75_1 | 84.55 | 0.14 |
| 76_1 | 102.91 | 13.06 |
| 77_1 | 99.31 | 8.11 |
| 78_1 | #N/A | #N/A |
| 79_1 | 55.12 | 5.20 |
| 80_1 | 90.80 | 2.66 |
| 80_2 | 98.59 | 2.30 |
| 81_1 | 67.03 | 2.60 |
| 82_1 | 59.63 | 2.52 |
| 82_2 | 68.54 | 1.17 |
| 83_1 | 64.43 | 2.61 |
| 83_2 | 64.11 | 0.89 |
| 84_1 | #N/A | #N/A |
| 85_1 | 96.19 | 0.06 |
| 85_2 | 90.23 | 8.54 |
| 86_1 | 89.93 | 1.33 |
| 86_2 | 106.93 | 12.98 |
| 87_1 | #N/A | #N/A |
| 88_1 | 33.65 | 2.65 |
| 89_1 | 95.60 | 3.51 |
| 90_1 | 97.32 | 6.73 |
| 91_1 | 61.96 | 5.80 |
| 92_1 | 64.15 | 10.92 |
| 93_1 | 92.01 | 6.19 |
| 94_1 | 96.44 | 10.38 |
| 95_1 | 80.17 | 2.61 |
| 95_2 | 77.56 | 1.32 |
| 96_1 | #N/A | #N/A |
| 97_1 | #N/A | #N/A |
| 98_1 | 107.81 | 13.88 |
| 99_1 | #N/A | #N/A |
| 100_1 | #N/A | #N/A |
| 101_1 | #N/A | #N/A |
| 102_1 | #N/A | #N/A |
| 103_1 | 114.99 | 24.45 |
| 104_1 | 103.45 | 5.92 |
| 105_1 | 75.27 | 1.94 |
| 105_2 | 61.11 | 2.24 |
| 106_1 | 101.58 | 1.06 |
| 106_2 | 101.83 | 5.69 |
| 107_1 | #N/A | #N/A |
| 108_1 | 103.24 | 5.94 |
| 108_2 | 101.74 | 5.49 |
| 108_3 | 90.13 | 13.66 |
| 109_1 | 100.11 | 4.80 |
| 109_2 | 113.58 | 2.84 |
| 110_1 | #N/A | #N/A |
| 111_1 | 92.73 | 1.91 |
| 112_1 | 105.20 | 2.71 |
| 113_1 | 103.59 | 5.78 |
| 114_1 | 103.47 | 7.90 |
| 115_1 | 107.24 | 8.20 |
| 116_1 | 85.06 | 7.26 |
| 116_2 | 80.29 | 5.56 |
| 117_1 | #N/A | #N/A |
| 117_2 | 95.10 | 2.33 |
| 117_3 | 96.89 | 13.60 |
| 118_1 | #N/A | #N/A |
| 119_1 | 85.82 | 4.56 |
| 120_1 | 85.68 | 11.44 |
| 120_2 | 93.79 | 5.14 |
| 121_1 | #N/A | #N/A |
| 122_1 | 144.58 | 44.88 |
| 122_2 | 113.11 | 8.57 |
| 123_1 | 59.62 | 9.47 |
| 124_1 | 52.57 | 2.90 |
| 125_1 | 101.47 | 7.25 |
| 126_1 | 129.31 | 9.43 |
| 127_1 | 87.40 | 4.43 |
| 128_1 | 80.60 | 7.16 |
| 129_1 | 95.26 | 0.97 |
| 130_1 | 125.56 | 4.29 |
| 131_1 | 107.17 | 5.92 |
| 132_1 | 21.86 | 2.92 |
| 133_1 | 77.50 | 10.35 |
| 134_1 | 106.91 | 0.79 |
| 135_1 | 103.33 | 1.13 |
| 136_1 | 64.06 | 3.46 |
| 137_1 | 70.57 | 14.52 |
| 138_1 | 70.14 | 17.00 |
| 139_1 | 62.23 | 1.13 |
| 139_2 | 59.93 | 2.52 |
| 140_1 | 90.30 | 7.69 |
| 141_1 | 63.42 | 2.15 |
| 142_1 | 123.38 | 47.71 |
| 143_1 | 69.90 | 3.87 |
| 144_1 | #N/A | #N/A |
| 145_1 | #N/A | #N/A |
| 145_2 | #N/A | #N/A |
| 146_1 | #N/A | #N/A |
| 147_1 | #N/A | #N/A |
| 147_2 | #N/A | #N/A |
| 148_1 | #N/A | #N/A |
| 149_1 | 107.30 | 13.12 |
| 150_1 | 112.39 | 27.25 |
| 151_1 | 72.22 | 6.20 |
| 152_1 | 128.68 | 44.31 |
| 153_1 | 148.38 | 11.62 |
| 153_2 | 136.75 | 4.21 |
| 154_1 | 99.55 | 5.83 |
| 155_1 | 110.71 | 8.09 |
| 156_1 | 116.06 | 0.75 |
| 157_1 | 106.15 | 16.19 |
| 158_1 | 98.71 | 16.36 |
| 158_2 | 85.55 | 5.15 |
| 159_1 | 118.35 | 1.48 |
| 160_1 | 165.75 | 9.61 |
| 161_1 | 158.90 | 0.88 |
| 162_1 | 104.97 | 1.85 |
| 163_1 | 133.83 | 9.44 |
| 164_1 | 84.46 | 17.61 |
| 165_1 | 95.10 | 11.69 |
| 166_1 | 74.96 | 8.17 |
| 167_1 | 68.09 | 11.24 |
| 168_1 | 90.07 | 16.56 |
| 169_1 | 92.53 | 8.41 |
| 169_2 | 102.95 | 9.23 |
| 169_3 | 100.23 | 4.07 |
| 170_1 | 100.71 | 10.54 |
| 171_1 | 120.58 | 39.11 |
| 172_1 | 113.47 | 9.82 |
| 173_1 | 102.28 | 2.81 |
| 174_1 | 87.69 | 3.76 |
| 175_1 | 122.11 | 5.30 |
| 176_1 | 69.43 | 2.44 |
| 177_1 | 108.37 | 7.16 |
| 178_1 | 107.27 | 9.14 |
| 178_2 | 117.93 | 3.77 |
| 179_1 | 114.12 | 5.05 |
| 180_1 | 93.24 | 2.68 |
| 181_1 | 98.66 | 2.53 |
| 182_1 | 89.46 | 7.87 |
| 183_1 | 83.55 | 4.22 |
| 184_1 | 70.62 | 12.67 |
| 185_1 | 97.55 | 10.88 |
| 186_1 | 96.65 | 8.31 |
| 187_1 | 54.10 | 8.19 |
| 188_1 | 101.60 | 8.41 |
| 189_1 | 68.00 | 10.70 |
| 190_1 | 141.71 | 16.49 |
| 190_2 | 83.85 | 6.76 |
| 191_1 | 148.53 | 8.91 |
| 192_1 | 106.29 | 5.85 |
| 192_2 | 85.20 | 10.09 |
| 193_1 | 91.71 | 5.71 |
| 193_2 | 73.38 | 2.71 |
| 194_1 | 93.19 | 4.43 |
| 195_1 | 95.70 | 2.72 |
| 196_1 | 67.40 | 8.02 |
| 197_1 | 114.76 | 21.86 |
| 198_1 | 85.13 | 6.45 |
| 199_1 | 91.59 | 3.24 |
| 200_1 | 87.64 | 6.54 |
| 201_1 | 101.76 | 5.01 |
| 201_2 | 110.01 | 8.04 |
| 202_1 | 85.95 | 4.63 |
| 202_2 | 88.94 | 3.52 |
| 203_1 | 92.92 | 2.20 |
| 203_2 | 104.16 | 3.17 |
| 203_3 | 129.86 | 20.13 |
| 204_1 | 112.00 | 27.06 |
| 205_1 | 87.02 | 9.91 |
| 206_1 | 90.88 | 10.42 |
| 206_2 | 67.71 | 14.83 |
| 207_1 | 93.70 | 3.15 |
| 208_1 | 90.05 | 3.01 |
| 209_1 | 69.85 | 4.14 |
| 210_1 | 86.07 | 4.68 |
| 210_2 | 84.28 | 15.59 |
| 211_1 | 101.07 | 4.83 |
| 212_1 | 94.44 | 4.57 |
| 213_1 | 99.59 | 14.01 |
| 214_1 | #N/A | #N/A |
| 215_1 | #N/A | #N/A |
| 216_1 | 96.46 | 10.44 |
| 216_2 | 103.47 | 4.95 |
| 217_1 | 104.08 | 4.88 |
| 218_1 | #N/A | #N/A |
| 219_1 | 112.81 | 6.71 |
| 220_1 | 93.55 | 8.40 |
| 221_1 | #N/A | #N/A |
| 221_2 | 93.70 | 5.32 |
| 221_3 | 106.21 | 8.49 |
| 222_1 | 97.90 | 13.52 |
| 222_2 | 103.67 | 13.27 |
| 223_1 | 78.11 | 5.49 |
| 224_1 | #N/A | #N/A |
| 225_1 | #N/A | #N/A |
| 226_1 | 98.77 | 6.85 |
| 227_1 | 132.53 | 18.49 |
| 228_1 | 90.41 | 3.32 |
| 229_1 | 107.49 | 3.69 |
| 229_2 | 92.21 | 3.07 |
| 230_1 | 93.65 | 2.24 |
| 230_2 | 105.60 | 5.47 |
| 231_1 | 97.51 | 5.97 |
| 232_1 | 111.24 | 3.86 |
| 233_1 | 119.06 | 18.13 |
| 234_1 | 85.73 | 12.74 |
| 234_2 | 101.48 | 9.74 |
| 234_3 | 91.68 | 23.71 |
| 235_1 | 93.48 | 9.09 |
| 236_1 | 95.73 | 8.67 |
| 237_1 | 87.22 | 5.14 |

#NA means that the CCK8 was not measured for the indicated compound.

The results show that of the 236 oligonucleotides in the library only 5% were not able to reduce at least either RTEL1 or cccDNA to at least 80% of the control. From this it can be seen that it is possible to produce oligonucleotides targeting across the entire RTEL1 transcript which are capable of reducing RTEL1 and/or cccDNA, generally the reduction in RTEL1 and cccDNA are not due to cell death, although it may be the case for a few of the compounds.

1. A RTEL1 inhibitor for use in the treatment and/or prevention of Hepatitis B virus (HBV) infection.

2. The RTEL1 inhibitor for use according to claim 1, wherein the HBV infection is a chronic infection.

3. The RTEL1 inhibitor for use according to claim 1 or 2, wherein the RTEL1 inhibitor is capable of reducing cccDNA in an infected cell.

4. The RTEL1 inhibitor for use according to any one of claims 1 to 3, wherein said inhibitor is an oligonucleotide of 12 to 60 nucleotides in length comprising a contiguous nucleotide sequence of at least 10 nucleotides in length which is at least 95% complementary to a mammalian RTEL1 target nucleic acid, in particular a human RTEL1 nucleic acid, and is capable of reducing RTEL1 mRNA.

5. The RTEL 1 inhibitor for use according to any one of claims 1 to 4 selected from a single stranded antisense oligonucleotide, siRNA or a shRNA molecule.

6. The RTEL 1 inhibitor for use according to any one of claims 1 to 5, wherein the mammalian RTEL1 target nucleic acid is selected from SEQ ID NO: 1 or 2.

7. The RTEL 1 inhibitor for use according to any one of claims 4 to 6, wherein the contiguous nucleotide sequence is at least 98% complementarity to the target nucleic acid of SEQ ID NO: 1 and SEQ ID NO: 2.

8. The RTEL 1 inhibitor for use according to any one of claims 3 to 7, wherein the cccDNA in an HBV infected cell is reduced by at least 60% when compared to a control.

9. The RTEL 1 inhibitor for use according to any one of claims 4 to 7, wherein the RTEL1 mRNA is reduced by at least 60% when compared to a control.

10. A single stranded antisense oligonucleotide of 12-30 nucleotides in length comprising a contiguous nucleotides sequence of at least 10 nucleotides which is complementary to a mammalian RTEL1, in particular a human RTEL1, wherein the oligonucleotide is capable of inhibiting the expression of RTEL1.

11. The antisense oligonucleotide according to claim 10, wherein the contiguous nucleotide sequence is at least 90% complementary, such as fully complementary, to SEQ ID NO: 1.

12. The antisense oligonucleotide according to claim 10 or 11 comprising a contiguous nucleotide sequence of 12 to 25, in particular 15 to 21 nucleotides in length.

13. An oligonucleotide according to any one of claims 11 to 12, wherein the contiguous nucleotide sequence is 100% complementary to a target sequence selected from SEQ ID NO: 3-21.

14. The oligonucleotide according to any one of claims 10 to 13, wherein the oligonucleotide comprises a sequence selected from the group consisting of SEQ ID NO: 22-237.

15. The antisense oligonucleotide according to any one of claims 10 to 14, comprising one or more 2′ sugar modified nucleoside.

16. The antisense oligonucleotide according to claim 15, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

17. The antisense oligonucleotide according to any one of claim 15 or 16, wherein the one or more 2′ sugar modified nucleoside is a LNA nucleoside.

18. The antisense oligonucleotide according to any one of claims 10 to 17, where the oligonucleotide comprises at least one phosphorothioate internucleoside linkage.

19. The antisense oligonucleotide according to claim 18, wherein all the internucleoside linkages within the contiguous nucleotide sequence are phosphorothioate internucleoside linkages.

20. The antisense oligonucleotide according to any one of claims 10 to 19, wherein the oligonucleotide is capable of recruiting RNase H.

21. The antisense oligonucleotide according to any one of claims 10 to 20, wherein the antisense oligonucleotide, or contiguous nucleotide sequence thereof, consists or comprises a gapmer of formula 5′-F-G-F′-3′, where region F and F′ independently comprise 1-4 2′ sugar modified nucleosides and G is a region between 6 and 16 nucleosides which are capable of recruiting RNaseH, such as a region comprising between 6 and 18 DNA nucleosides.

22. A conjugate comprising an oligonucleotide according to any one of claims 10 to 21 and at least one conjugate moiety covalently attached to said oligonucleotide.

23. The conjugate compound of claim 22, wherein the conjugate moiety is selected from one of the trivalent GalNAc moieties in FIG. 1.

24. The conjugate compound of claim 22 or 23 comprising a physiologically labile linker composed of 2 to 5 linked nucleosides comprising at least two consecutive phosphodiester linkages, wherein the physiologically labile linker covalently bound at the 5′ or 3′ terminal of the oligonucleotide component.

25. A pharmaceutically acceptable salt of an oligonucleotide according to any one of claims 10 to 21, or of a conjugate according to claims 22 to 24.

26. A pharmaceutical composition comprising an oligonucleotide according to any one of claims 10 to 21, or of a conjugate according to claims 22 to 24 or a pharmaceutically acceptable salt according to claim 25 and a pharmaceutically acceptable excipient.

27. An in vivo or in vitro method for modulating RTEL1 expression in a target cell which is expressing RTEL1, said method comprising administering an oligonucleotide according to any one of claims 10 to 21, or of a conjugate according to claims 22 to 24, a pharmaceutically acceptable salt according to claim 25, or a pharmaceutical composition according to claim 26 in an effective amount to said cell.

28. A method for treating or preventing a disease comprising administering a therapeutically or prophylactically effective amount of an oligonucleotide according any one of claims 10 to 21, or of a conjugate according to claims 22 to 24, a pharmaceutically acceptable salt according to claim 25, or a pharmaceutical composition according to claim 26, to a subject suffering from or susceptible to the disease.

29. A method according to claim 28, wherein the disease is Hepatitis B Virus (HBV).

30. An antisense oligonucleotide according any one of claims 10 to 21, or of a conjugate according to claims 22 to 24, a pharmaceutically acceptable salt according to claim 25, or a pharmaceutical composition according to claim 26 for use in medicine.

31. The use of an oligonucleotide according any one of claims 10 to 21, or of a conjugate according to claims 22 to 24, a pharmaceutically acceptable salt according to claim 25, or a pharmaceutical composition according to claim 26, for the preparation of a medicament for the treatment or prevention of Hepatitis B Virus HBV.
